(19) United States
(12) Patent Application Publication
(10) Pub. No.: US 2025/0277001 A1
(43) Pub. Date: Sep. 4, 2025

(54) PEPTIDE DERIVATIVES AND RELATED USES AS OREXIN AGONISTS

(71) Applicant: CENTESSA PHARMACEUTICALS (UK) LIMITED, Altrincham (GB)

(72) Inventors: Tomi K. SAWYER, SOUTHBOROUGH, MA (US); Noel Michael O'BOYLE, CAMBRIDGESHIRE (GB); Chris DE GRAAF, CAMBRIDGESHIRE (GB); John Andrew CHRISTOPHER, BEDFORDSHIRE (GB); Miles Stuart CONGREVE, MELBOURN (GB); Conor SCULLY, BROXBOURNE (GB)

(21) Appl. No.: 19/016,243

(22) Filed: Jan. 10, 2025

Related U.S. Application Data
(63) Continuation of application No. PCT/US2023/069969, filed on Jul. 11, 2023.
(60) Provisional application No. 63/388,131, filed on Jul. 11, 2022.

(51) Int. Cl. C07K 7/08

(52) U.S. Cl. CPC C07K 7/08

(57) ABSTRACT

The present disclosure relates to peptide having a sequence comprising:

Z₁X₁X₂X₃X₄X₅X₆X₇X₈X₉X₁₀X₁₁X₁₂X₁₃X₁₄X₁₅-NH₂
(Construct Number 1),

And to their isomers, pharmaceutically acceptable salts, or prodrugs thereof, methods of use, and methods for their preparation. The peptides disclosed herein are useful for modulating orexin receptor activity and may be used in the treatment of disorders in which orexin receptor activity is implicated, such as narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anaesthesia.

### RELATED APPLICATIONS

This application is a continuation of International Application No. PCT/US2023/069969, filed Jul. 11, 2023, which claims priority to U.S. Provisional Application No. 63/388,131, filed Jul. 11, 2022, the entire contents of which are incorporated herein by reference in their entireties.

### SEQUENCE LISTING

The instant application contains a Sequence Listing which has been submitted electronically in XML format and is hereby incorporated by reference in its entirety. Said XML copy, created on May 19, 2025, is named 60509-207301_SL_v2.xml and is 81,553 bytes in size.

### BACKGROUND

The present disclosure relates to small peptide orexin receptor agonists designed for the treatment of narcolepsy or other disorders associated with orexin insufficiency and/or excessive sleepiness. Narcolepsy afflicts 1 in 2000 individuals worldwide. Onset may occur during adolescence for a lifelong duration and debilitating impact on quality of life. Narcolepsy Type 1 (NT1) is caused by the loss of neurons in the brain which produce orexin neuropeptides. There is no known cure, and currently approved treatments are symptomatic and do not address the underlying cause of the disorder. Thus, development of pharmacotherapeutics to restore lost orexin signaling is critically important for treatment of the root cause of NT1.

In Narcolepsy Type 1, the sole population of neurons in the brain that produce orexin A and B (also known as hypocretin-1 and 2) peptides are destroyed by an immune-mediated mechanism which causes arousal state boundary dysfunction. Mouse models of narcolepsy type 1 recapitulate the orexin neurodegeneration and the two cardinal symptoms observed in NT1 patients, specifically excessive sleepiness and cataplexy-like events. Symptoms of narcolepsy type 1 and type 2 may include excessive daytime sleepiness, disturbed nighttime sleep, hypnagogic and hypnopompic hallucinations, and inappropriately timed rapid-eye-movement (REM) sleep. Cataplexy is the intrusion of sudden, reversible loss of muscle tone (the atonia of REM sleep) into wakefulness in response to emotional stimuli and is pathognomonic of NT1. Metabolic syndrome including insulin resistance is also observed in individuals with narcolepsy, particularly NT1.

The two predominant symptoms of narcolepsy type 1, excessive sleepiness and cataplexy, can be reduced by re-activation of orexin neurotransmission at OX2R. Reversal of cataplexy-like events and sleep/wake fragmentation has been achieved by genetic, focal restoration of OX2R signaling in the dorsal raphe nucleus of the pons and the tuberomammillary nucleus of the hypothalamus, respectively, in mice that otherwise lack orexin receptors in those regions. Intracerebroventricular (ICV) administration of orexin A (OXA) has been shown to increase time spent awake and decreases cataplexy-like behavior in orexin-neuron ablated mice. The selective OX2R agonist, YNT-185 administered intraperitoneally or ICV, modestly increases wakefulness in wild type (WT) and orexin ligand-deficient mice, and decreases sleep-onset REM periods and cataplexy-like events in an NT1 mouse model. Subcutaneous administration of TAK-925 modestly increased wakefulness in WT mice, but not in OX2R-knockout mice. Brain penetrant and stable OX2R agonists that are bioavailable after alternative routes of administration (including but not limited to oral, intranasal, transmucosal, and transdermal) and that bind with high affinity for potent excitation of arousal-state regulating neurons will provide an improvement to current therapeutics for patients with NT1. In fact, initial clinical studies reported with TAK-925 showed both substantial levels of increased wakefulness and trends for decreasing cataplexy in individuals with NT1. Activation of the OX1R is implicated in regulation of mood and reward behaviors, and may contribute to arousal.

Current therapies do not adequately address excessive sleepiness which is observed clinically in a range of indications. Orexin receptor agonists may also be useful in other indications marked by some degree of orexin neurodegeneration, decreased orexin levels (eg as measured in CSF), or decreased orexin signaling, and/or excessive daytime sleepiness, such as narcolepsy type 2, idiopathic hypersomnia, Parkinson's disease, Alzheimer's disease, Huntington's disease, Prader-Willi syndrome, multiple sclerosis, depression (including atypical depression and major depressive disorder), and traumatic brain injury. Orexin receptor agonists may also reduce excessive sleep, sleep paralysis, sleep inertia and sleep drunkenness in disorders such as narcolepsy and idiopathic hypersomnia. Because stimulation of OX2R promotes wakefulness in orexin-intact animals, orexin receptor agonists may treat or decrease excessive daytime sleepiness in patients (‘with normal’ or) without reduced levels of orexin, including narcolepsy type 2, idiopathic hypersomnia, sleep apnea and other underlying sleep disorders. Similarly, orexin receptor agonists may confer wake-promoting benefits in disorders of recurrent hypersomnia, such as Klein-Levin syndrome, or inappropriately timed sleep (i.e., circadian rhythm sleep disorders), such as delayed- or advanced-sleep phase disorder, shift work disorder and jet lag disorder, and excessive sleepiness caused by a disrupted sleep cycle. The abnormal daytime sleepiness, sleep onset REM periods, and cataplexy-like symptoms of some rare genetic disorders (e.g., ADCA-DN, Myotonic Dystrophy, Coffin-Lowry syndrome, Moebius syndrome, Norrie disease, Niemann-Pick disease type C, Smith-Magenis Syndrome (SMS), mucopolysaccharidoses (MPS) disorders, and Prader-Willi syndrome) could be alleviated with orexin receptor agonists. Other indications in which orexin receptor agonists have been suggested to confer benefits include attention deficit hyperactivity disorder, cognitive dysfunction related to age, disease or medications, metabolic syndrome and obesity, depression, fatigue, osteoporosis, cardiac failure, coma, and emergence from anesthesia.

The disclosure arises from a need to provide peptides for the modulation of orexin receptor activity in the brain, including activation of the orexin-2 receptor, with improved therapeutic potential. In particular, peptides with improved physicochemical, pharmacological and pharmaceutical properties to existing compounds are desirable.

### SUMMARY

In some aspects, the present disclosure provides a peptide having a sequence comprising:

Z₁X₁X₂X₃X₄X₅X₆X₇X₈X₉X₁₀X₁₁X₁₂X₁₃X₁₄X₁₅-NH₂
(Construct Number 1),

or an isomer, pharmaceutically acceptable salt, or prodrug thereof, wherein,
Z₁ is absent, a capping group, Arg, or hArg(Et)₂;
X₁ is absent, an amino acid comprising a hydrophobic side chain, or a derivative thereof, or a polar uncharged side chain, or a derivative thereof;
X₂ is an amino acid comprising a polar uncharged side chain, or a derivative thereof, a hydrophobic side chain, or a derivative thereof, or an electrically charged side chain, or a derivative thereof;
X₃ is an amino acid comprising an electrically charged side chain, or a derivative thereof, a polar uncharged side chain, or a derivative thereof, or a hydrophobic side chain, or a derivative thereof;
X₄ is an amino acid comprising a polar charged side chain, or a derivative thereof, or a hydrophobic side chain, or a derivative thereof;
X₅ is an amino acid comprising an electrically charged side chain, or a derivative thereof, or a polar uncharged side chain, or a derivative thereof;
X₆ is Gly or a Gly derivative;
X₇ is an amino acid comprising a polar uncharged side chain, or a derivative thereof;
X₈ is an amino acid comprising a polar charged side chain, or a derivative thereof, or a hydrophobic side chain, or a derivative thereof;
X₉ is an amino acid comprising a hydrophobic side chain, or a derivative thereof;
X₁₀ is Ala, Gly, or a Gly derivative;
X₁₁ is Gly or a Gly derivative;
X₁₂ is an amino acid comprising a hydrophobic side chain, or a derivative thereof;
X₁₃ is an amino acid comprising a hydrophobic side chain, or a derivative thereof;
X₁₄ is an amino acid comprising a polar uncharged side chain, or a derivative thereof; and
X₁₅ is an amino acid comprising a hydrophobic side chain or a derivative thereof.

In some aspects, the present disclosure provides a peptide having a sequence comprising:

Z₁X₁X₂X₃X₄X₅X₆X₇X₈X₉X₁₀X₁₁X₁₂X₁₃X₁₄X₁₅-NH₂
(Construct Number 2),

or an isomer, pharmaceutically acceptable salt, or prodrug thereof, wherein,
Z₁ is absent, a capping group, Arg, or hArg(Et)₂;
X₁ is absent, an amino acid comprising a hydrophobic side chain, or a derivative thereof, or a polar uncharged side chain, or a derivative thereof;
X₂ is an amino acid comprising a polar uncharged side chain, or a derivative thereof, a hydrophobic side chain, or a derivative thereof, or an electrically charged side chain, or a derivative thereof;
X₃ is an amino acid comprising an electrically charged side chain, or a derivative thereof, a polar uncharged side chain, or a derivative thereof, or a hydrophobic side chain, or a derivative thereof;
X₄ is an amino acid comprising a polar charged side chain, or a derivative thereof, or a hydrophobic side chain, or a derivative thereof;
X₅ is an amino acid comprising an electrically charged side chain, or a derivative thereof, or a polar uncharged side chain, or a derivative thereof;
X₆ is Gly or a Gly derivative;
X₇ is an amino acid comprising a polar uncharged side chain, or a derivative thereof;
X₈ is an amino acid comprising a polar charged side chain, or a derivative thereof, or a hydrophobic side chain, or a derivative thereof;
X₉ is an amino acid comprising a hydrophobic side chain, or a derivative thereof;
X₁₀ is Ala, Gly, or a Gly derivative;
X₁₁ is Gly or a Gly derivative;
X₁₂ is an amino acid comprising a hydrophobic side chain, or a derivative thereof;
X₁₃ is an amino acid comprising a hydrophobic side chain, or a derivative thereof;
X₁₄ is an amino acid comprising a polar uncharged side chain, or a derivative thereof; and
X₁₅ is 2-AOC, 2-AHP, NLE, NVA, Phe or Phe(3-Br), wherein the phenyl of X₁₅ is optionally substituted with halo, —OH, —O(C₁-C₆alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some aspects, the present disclosure provides a peptide having a sequence comprising:

Z₁X₁X₂X₃X₄X₅X₆X₇X₈X₉X₁₀X₁₁X₁₂X₁₃X₁₄X₁₅-NH₂
(Construct Number 3),

Z₁ is absent, a capping group, Arg, or hArg(Et)₂;
X₁ is absent, Phe, Leu, Gln, D-Gln, or Hyp(4-OH); X₂ is Gln, Asn, N-Me-Asn, Thr, Ser, D-Gln, D-Leu, Leu, hArg, D-hArg, or Hyp(4-OH);
X₃ is Arg, hArg(Et)₂, Hyp(4-OH), His, Lys, Asp, Glu, Ser, D-Phe, Trp, hArg, Pro, D-Pro, or D-hArg;
X₄ is Hyp(4-OH), His, Arg, Lys, Asp, Gln, Glu, Pro, D-Pro, Ala, or D-Ala;
X₅ is Arg, hArg(Et)₂, Hyp(4-OH), His, Lys, Asp, Glu, or Ser;
X₆ is N-Phenethyl-Gly, N-(naphthalen-2-yl-ethyl)-Gly, N-(naphthalen-1-yl-ethyl)-Gly, N-(3-EtNH₂-Phenethyl)-Gly, N-(4-OMe-Phenethyl)-Gly, Phg, Phg(4-OH), or Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₇ is Asn, N-Me-Asn, Ser, Thr, or Gln;
X₈ is Hyp(4-OH), His, Arg, Lys, Asp, Gln, or Glu;
X₉ is Ala, Val, Ile, Leu, Met, Phe, Tyr, or Trp, wherein the phenyl of X₉ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₀ is Phg, Phg(4-OH), N-Phenethyl-Gly, N(naphtha-2-yl-ethyl)-Gly, N-(4-OMe-Phenethyl)-Gly, Gly, or Ala, wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₁ is Phg, Phg(4-OH), N-Phenethyl-Gly, N(naphtha-2-yl-ethyl)-Gly, N-(4-OMe-Phenethyl)-Gly, or Gly, wherein the phenyl of X₁₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₂ is Ile, Ala, Val, Leu, Met, Phe, Tyr, or Trp, wherein the phenyl of X₁₂ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₃ is N-Me-Leu, Leu, Ala, Val, Ile, Met, Phe, Tyr, or Trp, wherein the phenyl of X₁₃ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₄ is Thr, Ser, Asn, or Gln; and
X₁₅ is 2-AOC, 2-AHP, NLE, NVA, Phe, or Phe(3-Br), wherein the phenyl of X₁₅ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some aspects, the present disclosure provides Construct Number 3, wherein:
Z₁ is absent or CAP;
X₁ is absent, Phe, Leu, Gln, D-Gln, or Hyp(4-OH);
X₂ is Gln, D-Gln, Hyp(4-OH), D-Leu, hArg, D-hArg, or Leu;
X₃ is Hyp(4-OH), D-Phe, Trp, hArg, Pro, D-Pro, or D-hArg;
X₄ is Hyp(4-OH), Arg, Pro, D-Pro, Ala, or D-Ala;
X₅ is Arg, hArg(Et)₂, Lys, or Ser;
X₆ is N-Phenethyl-Gly or Gly;
X₇ is Asn or N-Me-Asn;
X₈ is Hyp(4-OH) or His;
X₉ is Ala;
X₁₀ is Phg or Ala;
X₁₁ is Gly;
X₁₂ is Ile;
X₁₃ is Leu;
X₁₄ is Thr; and
X₁₅ is 2-AOC or NLE.

In some aspects, the present disclosure provides a peptide obtainable by, or obtained by, a method described herein.

In some aspects, the present disclosure provides a pharmaceutical composition comprising the peptide disclosed herein or an isomer, pharmaceutically acceptable salt, or prodrug thereof, and a pharmaceutically acceptable diluent or carrier.

In some aspects, the present disclosure provides a method of modulating orexin-2 receptor activity, comprising contacting a cell with an effective amount of the peptide disclosed herein.

In some aspects, the present disclosure provides a method of treating or preventing a disease or disorder in a subject in need thereof, comprising administering to the subject a therapeutically effective amount of the peptide or pharmaceutical composition disclosed herein.

In some aspects, the present disclosure provides a peptide or pharmaceutical composition disclosed herein for use in modulating orexin-2 receptor activity; optionally, the activity is in vitro or in vivo.

In some aspects, the present disclosure provides a peptide or pharmaceutical composition disclosed herein for use in treating or preventing a disease or disorder.

In some aspects, the present disclosure provides a use of the peptide disclosed herein in the manufacture of a medicament for modulating orexin-2 receptor activity.

In some aspects, the present disclosure provides a use of the peptide disclosed herein in the manufacture of a medicament for treating or preventing a disease or disorder.

In some aspects, the present disclosure provides a method, use, or peptide or pharmaceutical composition for use of the peptide disclosed herein in combination with one or more additional therapy.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the peptides disclosed herein, the chemical structures will control.

Other features and advantages of the disclosure will be apparent from the following detailed description and claims.

### DETAILED DESCRIPTION

The present disclosure relates to peptide derivatives, prodrugs, and pharmaceutically acceptable salts thereof, which may modulate orexin receptor activity and are accordingly useful in methods of treatment of the human or animal body. The present disclosure also relates to processes for the preparation of these peptides, to pharmaceutical compositions comprising them and to their use in the treatment of disorders in which the orexin receptor is implicated, such as narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options. For example, while various options for variables X, L, and Y are described herein, the disclosure may be interpreted as excluding structures for non-operable peptides caused by certain combinations of variables X, L, and Y (e.g., when each of X, L, and Y is —O—).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl). In some embodiments, the heteroaryl is thiophenyl or benzothiophenyl. In some embodiments, the heteroaryl is thiophenyl. In some embodiments, the heteroaryl benzothiophenyl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable peptide. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable peptide” and “stable structure” are meant to indicate a peptide that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the peptide of a given formula, then such substituent may be bonded via any atom in such formula. Combinations of substituents and/or variables are permissible, but only if such combinations result in stable peptides.

When any variable (e.g., R) occurs more than one time in any constituent or formula for a peptide, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable peptides.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the term “hArg(Et)₂” refers to

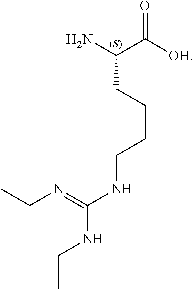

As used herein, the term “hArg” refers to

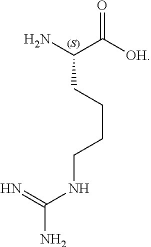

As used herein, the term “2-AOC” refers to

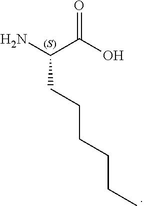

As used herein, the term “2-AHP” refers to

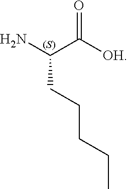

As used herein, the term “NVA” refers to

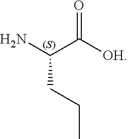

As used herein, the term “Phe(3-Br)” refers to

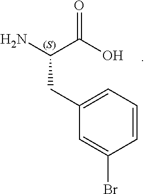

As used herein, the term “NLE” refers to

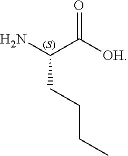

As used herein, the term “Phg” refers to

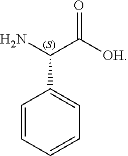

As used herein, the term “Phg(4-OH)” refers to

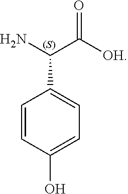

As used herein, the term “Nag(1)” refers to

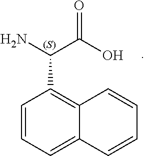

As used herein, the term “Phg(4-OMe)” refers to

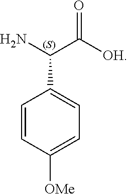

As used herein, the term “Phg(3-OH)” refers to

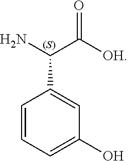

As used herein, the term “Hyp(4-OH)” refers to

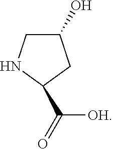

As used herein, the term “N-Me-Asn” refers to

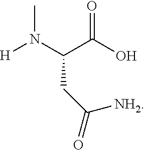

As used herein, the term “N-Phenethyl-Gly” refers to

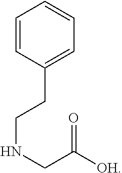 [SMILES: O=C(O)CNCCc1ccccc1]

As used herein, the term “N(naphthalen-2-yl-ethyl)-Gly” refers to

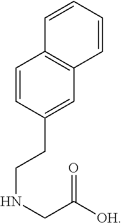 [SMILES: O=C(O)CNCCc1ccc2ccccc2c1]

As used herein, the term “N(naphthalen-1-yl-ethyl)-Gly” refers to

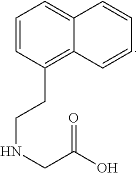 [SMILES: O=C(O)CNCCc1cccc2ccccc12]

As used herein, the term “N-(4-OMe-Phenethyl)-Gly” refers to

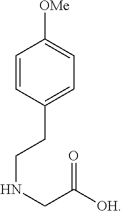 [SMILES: COc1ccc(CCNCC(=O)O)cc1]

As used herein, the term “N-(3-EtNH₂-Phenethyl)-Gly” refers to

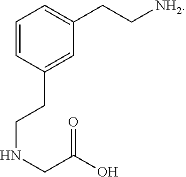 [SMILES: NCCc1cccc(CCNCC(=O)O)c1]

It is to be understood that the term “polar uncharged side chain” refers to a hydrophilic moiety that can hydrogen bond with another molecule at physiological pH.

It is to be understood that the term “electrically charged side chain” refers to a charged moiety (e.g., negative or positive) at physiological pH.

It is to be understood that the term “polar charged side chain” refers to a charged moiety that can hydrogen bond with another molecule at physiological pH.

It is to be understood that the term “hydrophobic side chain” refers to a moiety with a small dipole moments that may be repelled from water.

It is well known by those of ordinary skill in the art that certain amino acid substitutions can occur between amino acids with similar properties (i.e. classifications). For example, an amino acid classified as “hydrophobic” may be substituted with a different amino acid classified as “hydrophobic” when the hydrophobic property is desired at a position in an amino acid sequence. Other classifications may be used in a similar manner in amino acid substitutions, such as charge classification (e.g. neutral, positive, or negative), hydrophobic, hydrophilic, polar, non-polar, acidic, basic, aliphatic, and aromatic amino acids. It is understood that amino acids can have one or more categorization. For example, an amino acid may be “polar charged” or “polar uncharged.” Substitution of one amino acid for another of the same classification is contemplated for the amino acid sequences described herein. Illustrative, non-limiting, amino acid categorizations are summarized below:

| | | | | Charge |
|---|---|---|---|---|
| | Three Letter | One Letter | Polar/Nonpolar | Classifi- |
| Amino Acid | Abbreviation | Abbreviation | Classification | cation |
| Alanine | Ala | A | nonpolar | neutral |
| Arginine | Arg | R | polar | positive |
| Asparagine | Asn | N | polar | neutral |
| Aspartic Acid | Asp | D | polar | negative |
| Cysteine | Cys | C | nonpolar | neutral |
| Glutamic acid | Glu | E | polar | negative |
| Glutamine | Gln | Q | polar | neutral |
| Glycine | Gly | G | nonpolar | neutral |
| Histidine | His | H | polar | positive |
| Isoleucine | Ile | I | nonpolar | neutral |
| Leucine | Leu | L | nonpolar | neutral |
| Lysine | Lys | K | polar | positive |
| Methionine | Met | M | nonpolar | neutral |
| Phenylalanine | Phe | F | nonpolar | neutral |
| Proline | Pro | P | nonpolar | neutral |
| Serine | Ser | S | polar | neutral |
| Threonine | Thr | T | polar | neutral |
| Tryptophan | Trp | W | nonpolar | neutral |
| Tyrosine | Tyr | Y | polar | neutral |
| Valine | Val | V | nonpolar | neutral |

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the peptides of any of the Formulae described herein. The present disclosure also provides detailed methods for the synthesis of various disclosed peptides of the present disclosure according to the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final peptide at or near the end of the overall process, although it may be desirable in certain instances to further convert the peptide to a pharmaceutically acceptable salt thereof.

It is to be understood that peptides of the present disclosure can be prepared in a variety of ways using commercially available starting materials, peptides known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for organic Synthesis, John Wiley and Sons (1994); and L. Paquette, ed., Encyclopedia of Reagents for organic Synthesis, John Wiley and Sons (1995), incorporated by reference herein, are useful and recognised reference textbooks of organic synthesis known to those in the art

One of ordinary skill in the art will note that, during the reaction sequences and synthetic routes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the peptides to provide such treatment or prevention as is described herein. It is to be further understood, unless otherwise stated, any description of a method of treatment or prevention includes use of the peptides to prepare a medicament to treat or prevent such condition. The treatment or prevention includes treatment or prevention of human or non-human animals including rodents and other disease models.

It is to be understood that, unless otherwise stated, any description of a method of treatment includes use of the peptides to provide such treatment as is described herein. It is to be further understood, unless otherwise stated, any description of a method of treatment includes use of the peptides to prepare a medicament to treat such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In some embodiments, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a peptide of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a peptide of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any peptide described herein in combination with at least one pharmaceutically acceptable excipient or carrier.

As used herein, the term “pharmaceutical composition” is a formulation containing the peptides of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed peptide or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a peptide of this disclosure include powders, sprays, ointments, pastes, creams, lotions, gels, solutions, patches and inhalants. In some embodiments, the active peptide is mixed under sterile conditions with a pharmaceutically acceptable carrier, and with any preservatives, buffers, or propellants that are required.

As used herein, the term “pharmaceutically acceptable” refers to those peptides, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a peptide or pharmaceutical composition of the disclosure can be administered to a subject in many of the well-known methods currently used for chemotherapeutic treatment. For example, a peptide of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any peptide, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active peptides of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active peptides into preparations that can be used pharmaceutically. The appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active peptide in the required amount in an appropriate solvent with one or a combination of ingredients enumerated above, as required, followed by filtered sterilisation. Generally, dispersions are prepared by incorporating the active peptide into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active peptide can be incorporated with excipients and used in the form of tablets, troches, or capsules. Oral compositions can also be prepared using a fluid carrier for use as a mouthwash, wherein the peptide in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or peptides of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

For administration by inhalation, the peptides are delivered in the form of an aerosol spray from pressured container or dispenser, which contains a suitable propellant, e.g., a gas such as carbon dioxide, or a nebuliser.

For intranasal administration, the peptides are delivered in solution or solid formulation. In some embodiments, the peptides are delivered in solution as a mist, a drip, or a swab. In some embodiments, the peptides are delivered as a powder. In some embodiments, the peptide is included in a kit which further includes an intranasal applicator.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active peptides are formulated into ointments, salves, gels, or creams as generally known in the art.

The active peptides can be prepared with pharmaceutically acceptable carriers that will protect the peptide against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active peptide calculated to produce the desired therapeutic effect in association with the required pharmaceutical carrier. The specification for the dosage unit forms of the disclosure are dictated by and directly dependent on the unique characteristics of the active peptide and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active peptide to produce the desired biological effect in a subject or cell.

It is to be understood that the pharmaceutical compositions can be included in a container, pack, or dispenser together with instructions for administration.

It is to be understood that, for the peptides of the present disclosure being capable of further forming salts, all of these forms are also contemplated within the scope of the claimed disclosure.

As used herein, the term “pharmaceutically acceptable salts” refer to derivatives of the peptides of the present disclosure wherein the parent peptide is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent peptide formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent peptide either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the peptide to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The peptides, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In some embodiments, the peptide is administered orally. One skilled in the art will recognise the advantages of certain routes of administration.

The dosage regimen utilising the peptides is selected in accordance with a variety of factors including type, species, age, weight, sex and medical condition of the patient; the severity of the condition to be treated; the route of administration; the renal and hepatic function of the patient; and the particular peptide or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed peptides of the disclosure can be found in Remington: the Science and Practice of Pharmacy, 19^{th} edition, Mack Publishing Co., Easton, PA (1995). In some embodiments, the peptides described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The peptides will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic routes described herein, peptides may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “peptide of the disclosure” refers to those peptides which are disclosed herein, both generically and specifically.

### Peptides of the Present Disclosure

In some aspects, the present disclosure provides a peptide having a sequence comprising:

Z₁X₁X₂X₃X₄X₅X₆X₇X₈X₉X₁₀X₁₁X₁₂X₁₃X₁₄X₁₅-NH₂
(Construct Number 1),

or an isomer, pharmaceutically acceptable salt, or prodrug thereof, wherein,
Z₁ is absent, a capping group, Arg, or hArg(Et)₂;
X₁ is absent, an amino acid comprising a hydrophobic side chain, or a derivative thereof, or a polar uncharged side chain, or a derivative thereof;
X₂ is an amino acid comprising a polar uncharged side chain, or a derivative thereof, a hydrophobic side chain, or a derivative thereof, or an electrically charged side chain, or a derivative thereof;
X₃ is an amino acid comprising an electrically charged side chain, or a derivative thereof, a polar uncharged side chain, or a derivative thereof, or a hydrophobic side chain, or a derivative thereof;
X₄ is an amino acid comprising a polar charged side chain, or a derivative thereof, or a hydrophobic side chain, or a derivative thereof;
X₅ is an amino acid comprising an electrically charged side chain, or a derivative thereof, or a polar uncharged side chain, or a derivative thereof;
X₆ is Gly or a Gly derivative;
X₇ is an amino acid comprising a polar uncharged side chain, or a derivative thereof;
X₈ is an amino acid comprising a polar charged side chain, or a derivative thereof, or a hydrophobic side chain, or a derivative thereof;
X₉ is an amino acid comprising a hydrophobic side chain, or a derivative thereof;
X₁₀ is Ala, Gly, or a Gly derivative;
X₁₁ is Gly or a Gly derivative;
X₁₂ is an amino acid comprising a hydrophobic side chain, or a derivative thereof;
X₁₃ is an amino acid comprising a hydrophobic side chain, or a derivative thereof;
X₁₄ is an amino acid comprising a polar uncharged side chain, or a derivative thereof; and
X₁₅ is an amino acid comprising a hydrophobic side chain or a derivative thereof.

In some aspects, the present disclosure provides a peptide having a sequence comprising:

Z₁X₁X₂X₃X₄X₅X₆X₇X₈X₉X₁₀X₁₁X₁₂X₁₃X₁₄X₁₅-NH₂
(Construct Number 2),

or an isomer, pharmaceutically acceptable salt, or prodrug thereof, wherein,
Z₁ is absent, a capping group, Arg, or hArg(Et)₂;
X₁ is absent, an amino acid comprising a hydrophobic side chain, or a derivative thereof, or a polar uncharged side chain, or a derivative thereof;
X₂ is an amino acid comprising a polar uncharged side chain, or a derivative thereof, a hydrophobic side chain, or a derivative thereof, or an electrically charged side chain, or a derivative thereof;
X₃ is an amino acid comprising an electrically charged side chain, or a derivative thereof, a polar uncharged side chain, or a derivative thereof, or a hydrophobic side chain, or a derivative thereof;
X₄ is an amino acid comprising a polar charged side chain, or a derivative thereof, or a hydrophobic side chain, or a derivative thereof;
X₅ is an amino acid comprising an electrically charged side chain, or a derivative thereof, or a polar uncharged side chain, or a derivative thereof;
X₆ is Gly or a Gly derivative;
X₇ is an amino acid comprising a polar uncharged side chain, or a derivative thereof;
X₈ is an amino acid comprising a polar charged side chain, or a derivative thereof, or a hydrophobic side chain, or a derivative thereof;
X₉ is an amino acid comprising a hydrophobic side chain, or a derivative thereof;
X₁₀ is Ala, Gly, or a Gly derivative;
X₁₁ is Gly or a Gly derivative;
X₁₂ is an amino acid comprising a hydrophobic side chain, or a derivative thereof;
X₁₃ is an amino acid comprising a hydrophobic side chain, or a derivative thereof;
X₁₄ is an amino acid comprising a polar uncharged side chain, or a derivative thereof; and
X₁₅ is 2-AOC, 2-AHP, NLE, NVA, Phe or Phe(3-Br), wherein the phenyl of X₁₅ is optionally substituted with halo, —OH, —O(C₁-C₆alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some aspects, the present disclosure provides a peptide having a sequence comprising:

Z₁X₁X₂X₃X₄X₅X₆X₇X₈X₉X₁₀X₁₁X₁₂X₁₃X₁₄X₁₅-NH₂
(Construct Number 3),

Z₁ is absent, a capping group, Arg, or hArg(Et)₂;
X₁ is absent, Phe, Leu, Gln, D-Gln, or Hyp(4-OH);
X₂ is Gln, Asn, N-Me-Asn, Thr, Ser, D-Gln, D-Leu, Leu, hArg, D-hArg, or Hyp(4-OH);
X₃ is Arg, hArg(Et)₂, Hyp(4-OH), His, Lys, Asp, Glu, Ser, D-Phe, Trp, hArg, Pro, D-Pro, or D-hArg;
X₄ is Hyp(4-OH), His, Arg, Lys, Asp, Gln, Glu, Pro, D-Pro, Ala, or D-Ala;
X₅ is Arg, hArg(Et)₂, Hyp(4-OH), His, Lys, Asp, Glu, or Ser;
X₆ is N-Phenethyl-Gly, N-(naphthalen-2-yl-ethyl)-Gly, N-(naphthalen-1-yl-ethyl)-Gly, N-(3-EtNH₂-Phenethyl)-Gly, N-(4-OMe-Phenethyl)-Gly, Phg, Phg(4-OH), or Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₇ is Asn, N-Me-Asn, Ser, Thr, or Gln;
X₈ is Hyp(4-OH), His, Arg, Lys, Asp, Gln, or Glu;
X₉ is Ala, Val, Ile, Leu, Met, Phe, Tyr, or Trp, wherein the phenyl of X₉ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₀ is Phg, Phg(4-OH), N-Phenethyl-Gly, N(naphtha-2-yl-ethyl)-Gly, N-(4-OMe-Phenethyl)-Gly, Gly, or Ala, wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₁ is Phg, Phg(4-OH), N-Phenethyl-Gly, N(naphtha-2-yl-ethyl)-Gly, N-(4-OMe-Phenethyl)-Gly, or Gly, wherein the phenyl of X₁₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₂ is Ile, Ala, Val, Leu, Met, Phe, Tyr, or Trp, wherein the phenyl of X₁₂ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₃ is N-Me-Leu, Leu, Ala, Val, Ile, Met, Phe, Tyr, or Trp, wherein the phenyl of X₁₃ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₄ is Thr, Ser, Asn, or Gln; and
X₁₅ is 2-AOC, 2-AHP, NLE, NVA, Phe, or Phe(3-Br), wherein the phenyl of X₁₅ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some aspects, the present disclosure provides Construct Number 3, wherein:
Z₁ is absent or CAP;
X₁ is absent, Phe, Leu, Gln, D-Gln, or Hyp(4-OH);
X₂ is Gln, D-Gln, Hyp(4-OH), D-Leu, hArg, D-hArg, or Leu;
X₃ is Hyp(4-OH), D-Phe, Trp, hArg, Pro, D-Pro, or D-hArg;
X₄ is Hyp(4-OH), Arg, Pro, D-Pro, Ala, or D-Ala;
X₅ is Arg, hArg(Et)₂, Lys, or Ser;
X₆ is N-Phenethyl-Gly or Gly;
X₇ is Asn or N-Me-Asn;
X₈ is Hyp(4-OH) or His;
X₉ is Ala;
X₁₀ is Phg or Ala;
X₁₁ is Gly;
X₁₂ is Ile;
X₁₃ is Leu;
X₁₄ is Thr; and
X₁₅ is 2-AOC or NLE.

It is understood that, for a peptide of the present disclosure, variables Z₁, X₁, X₂, X₃, X₄, X₅, X₆, X₇, X₈, X₉, X₁₀, X₁₁, X₁₂, X₁₃, X₁₄, or X₁₅ can each be, where applicable, selected from the groups described herein, and any group described herein for any of variables Z₁, X₁, X₂, X₃, X₄, X₅, X₆, X₇, X₈, X₉, X₁₀, X₁₁, X₁₂, X₁₃, X₁₄, or X₁₅ can be combined, where applicable, with any group described herein for one or more of the remainder of variables Z₁, X₁, X₂, X₃, X₄, X₅, X₆, X₇, X₈, X₉, X₁₀, X₁₁, X₁₂, X₁₃, X₁₄, or X₁₅.

In some embodiments, Z₁ is absent, a capping group, Arg, or hArg(Et)₂.

In some embodiments, Z₁ is a capping group, Arg, or hArg(Et)₂.

In some embodiments, Z₁ is absent.

In some embodiments, Z₁ is a capping group.

In some embodiments, Z₁ is Arg or hArg(Et)₂.

In some embodiments, Z₁ is Arg. In some embodiments, Z₁ is hArg(Et)₂.

In some embodiments, X₁ is absent, an amino acid comprising a hydrophobic side chain, or a derivative thereof, or a polar uncharged side chain, or a derivative thereof.

In some embodiments, X₁ is absent.

In some embodiments, X₁ is an amino acid comprising a hydrophobic side chain, or a derivative thereof.

In some embodiments, X₁ is an amino acid comprising a hydrophobic side chain.

In some embodiments, X₁ is an amino acid comprising a polar uncharged side chain, or a derivative thereof.

In some embodiments, X₁ is an amino acid comprising a polar uncharged side chain.

In some embodiments, X₁ is absent, Phe, Leu, Gln, D-Gln, or Hyp(4-OH).

In some embodiments, X₁ is absent, Phe, Leu, or Hyp(4-OH).

In some embodiments, X₁ is Phe, Leu, or Hyp(4-OH).

In some embodiments, X₁ is Phe. In some embodiments, X₁ is Leu. In some embodiments, X₁ is or Hyp(4-OH).

In some embodiments, X₁ is Gln or D-Gln.

In some embodiments, X₁ is Gln. In some embodiments, X₁ is D-Gln.

In some embodiments, X₂ is an amino acid comprising a polar uncharged side chain, or a derivative thereof.

In some embodiments, X₂ is an amino acid comprising a polar uncharged side chain.

In some embodiments, X₂ is Gln, Asn, N-Me-Asn, Thr, Ser, D-Gln, D-Leu, Leu, hArg, D-hArg, or Hyp(4-OH).

In some embodiments, X₂ is Gln, Asn, N-Me-Asn, Thr, Ser, D-Gln, D-Leu, Leu, or Hyp(4-OH).

In some embodiments, X₂ is Gln, D-Gln, Hyp(4-OH), D-Leu, or Leu.

In some embodiments, X₂ is Gln. In some embodiments, X₂ is Asn. In some embodiments, X₂ is N-Me-Asn. In some embodiments, X₂ is Thr. In some embodiments, X₂ is Ser. In some embodiments, X₂ is D-Gln. In some embodiments, X₂ is D-Leu. In some embodiments, X₂ is Leu. In some embodiments, X₂ is Hyp(4-OH).

In some embodiments, X₂ is hArg or D-hArg.

In some embodiments, X₂ is hArg. In some embodiments, X₂ is D-hArg.

In some embodiments, X₃ is an amino acid comprising an electrically charged side chain, or a derivative thereof, a polar uncharged side chain, or a derivative thereof, or a hydrophobic side chain, or a derivative thereof.

In some embodiments, X₃ is an amino acid comprising an electrically charged side chain, or a derivative thereof, or a polar uncharged side chain, or a derivative thereof.

In some embodiments, X₃ is an amino acid comprising an electrically charged side chain, or a derivative thereof.

In some embodiments, X₃ is an amino acid comprising an electrically charged side chain.

In some embodiments, X₃ is an amino acid comprising a polar uncharged side chain, or a derivative thereof.

In some embodiments, X₃ is an amino acid comprising a polar uncharged side chain.

In some embodiments, X₃ is an amino acid comprising a hydrophobic side chain, or a derivative thereof.

In some embodiments, X₃ is an amino acid comprising a hydrophobic side chain.

In some embodiments, X₃ is an amino acid comprising a derivative of a hydrophobic side chain.

In some embodiments, X₃ is Hyp(4-OH), D-Phe, Trp, hArg, or D-hArg.

In some embodiments, X₃ is Arg, hArg(Et)₂, Hyp(4-OH), His, Lys, Asp, Glu, Ser, D-Phe, Trp, hArg, or D-hArg.

In some embodiments, X₃ is Arg, hArg(Et)₂, Hyp(4-OH), His, Lys, Asp, Glu, Ser, D-Phe, Trp, hArg, D-hArg, Pro, or D-Pro.

In some embodiments, X₃ is Arg. In some embodiments, X₃ is hArg(Et)₂. In some embodiments, X₃ is Hyp(4-OH). In some embodiments, X₃ is His. In some embodiments, X₃ is Lys. In some embodiments, X₃ is Asp. In some embodiments, X₃ is Glu. In some embodiments, X₃ is Ser. In some embodiments, X₃ is D-Phe. In some embodiments, X₃ is Trp. In some embodiments, X₃ is hArg. In some embodiments, X₃ is D-hArg.

In some embodiments, X₃ is Pro or D-Pro.

In some embodiments, X₃ is Pro. In some embodiments, X₃ is D-Pro.

In some embodiments, X₄ is an amino acid comprising a polar charged side chain, or a derivative thereof, or a hydrophobic side chain, or a derivative thereof.

In some embodiments, X₄ is an amino acid comprising a polar charged side chain, or a derivative thereof.

In some embodiments, X₄ is an amino acid comprising a polar charged side chain.

In some embodiments, X₄ is an amino acid comprising a hydrophobic side chain, or a derivative thereof.

In some embodiments, X₄ is an amino acid comprising a hydrophobic side chain.

In some embodiments, X₄ is an amino acid comprising a derivative of a hydrophobic side chain.

In some embodiments, X₄ is Hyp(4-OH), His, Arg, Lys, Asp, Gln, Glu, Pro, D-Pro, Ala, or D-Ala.

In some embodiments, X₄ is Hyp(4-OH), Arg, Pro, D-Pro, Ala, or D-Ala.

In some embodiments, X₄ is Hyp(4-OH). In some embodiments, X₄ is His. In some embodiments, X₄ is Arg. In some embodiments, X₄ is Lys. In some embodiments, X₄ is Asp. In some embodiments, X₄ is Gln. In some embodiments, X₄ is Glu. In some embodiments, X₄ is Pro. In some embodiments, X₄ is D-Pro. In some embodiments, X₄ is Ala. In some embodiments, X₄ is D-Ala.

In some embodiments, X₅ is an amino acid comprising an electrically charged side chain, or a derivative thereof, or a polar uncharged side chain, or a derivative thereof.

In some embodiments, X₅ is an amino acid comprising an electrically charged side chain, or a derivative thereof.

In some embodiments, X₅ is an amino acid comprising an electrically charged side chain.

In some embodiments, X₅ is an amino acid comprising a polar uncharged side chain, or a derivative thereof.

In some embodiments, X₅ is an amino acid comprising a polar uncharged side chain.

In some embodiments, X₅ is Arg, hArg(Et)₂, Hyp(4-OH), His, Lys, Asp, Glu, or Ser.

In some embodiments, X₅ is Arg, hArg(Et)₂, Lys, or Ser.

In some embodiments, X₅ is Arg. In some embodiments, X₅ is hArg(Et)₂. In some embodiments, X₅ is Hyp(4-OH). In some embodiments, X₅ is His. In some embodiments, X₅ is Lys. In some embodiments, X₅ is Asp. In some embodiments, X₅ is Glu. In some embodiments, X₅ is Ser.

In some embodiments, X₆ is Gly or a Gly derivative.

In some embodiments, X₆ is Gly.

In some embodiments, X₆ is a Gly derivative.

In some embodiments, X₆ is N-Phenethyl-Gly, N-(naphthalen-2-yl-ethyl)-Gly, N-(naphthalen-1-yl-ethyl)-Gly, N-(3-EtNH₂-Phenethyl)-Gly, N-(4-OMe-Phenethyl)-Gly, Phg, Phg(4-OH), or Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₆ is N-Phenethyl-Gly, N-(naphthalen-2-yl-ethyl)-Gly, N-(naphthalen-1-yl-ethyl)-Gly, N-(3-EtNH₂-Phenethyl)-Gly, N-(4-OMe-Phenethyl)-Gly, Phg, Phg(4-OH), or Gly.

In some embodiments, X₆ is N-Phenethyl-Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₆ is N-Phenethyl-Gly or Gly.

In some embodiments, X₆ is N-Phenethyl-Gly.

In some embodiments, X₆ is N-(naphthalen-2-yl-ethyl)-Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₆ is N-(naphthalen-2-yl-ethyl)-Gly.

In some embodiments, X₆ is N-(naphthalen-1-yl-ethyl)-Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₆ is N-(naphthalen-1-yl-ethyl)-Gly.

In some embodiments, X₆ is N-(3-EtNH₂-Phenethyl)-Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₆ is N-(3-EtNH₂-Phenethyl)-Gly.

In some embodiments, X₆ is N-(4-OMe-Phenethyl)-Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₆ is N-(4-OMe-Phenethyl)-Gly.

In some embodiments, X₆ is Phg, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₆ is Phg.

In some embodiments, X₆ is Phg(4-OH), wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₆ is Phg(4-OH).

In some embodiments, X₆ is Gly.

In some embodiments, X₇ is an amino acid comprising a polar uncharged side chain, or a derivative thereof.

In some embodiments, X₇ is an amino acid comprising a polar uncharged side chain.

In some embodiments, X₇ is Asn, N-Me-Asn, Ser, Thr, or Gln.

In some embodiments, X₇ is Asn or N-Me-Asn.

In some embodiments, X₇ is Asn. In some embodiments, X₇ is N-Me-Asn. In some embodiments, X₇ is Ser. In some embodiments, X₇ is Thr. In some embodiments, X₇ is Gln.

In some embodiments, X₈ is an amino acid comprising a polar charged side chain, or a derivative thereof, or a hydrophobic side chain, or a derivative thereof.

In some embodiments, X₈ is an amino acid comprising a polar charged side chain, or a derivative thereof.

In some embodiments, X₈ is an amino acid comprising a polar charged side chain.

In some embodiments, X₈ is an amino acid comprising a hydrophobic side chain, or a derivative thereof.

In some embodiments, X₈ is an amino acid comprising a hydrophobic side chain.

In some embodiments, X₈ is an amino acid comprising a derivative of a hydrophobic side chain.

In some embodiments, X₈ is Hyp(4-OH), His, Arg, Lys, Asp, Gln, or Glu.

In some embodiments, X₈ is Hyp(4-OH) or His.

In some embodiments, X₈ is Hyp(4-OH). In some embodiments, X₈ is His. In some embodiments, X₈ is Arg. In some embodiments, X₈ is Lys. In some embodiments, X₈ is Asp. In some embodiments, X₈ is Gln. In some embodiments, X₈ is Glu.

In some embodiments, X₉ is an amino acid comprising a hydrophobic side chain, or a derivative thereof.

In some embodiments, X₉ is an amino acid comprising a hydrophobic side chain.

In some embodiments, X₉ is Ala, Val, Ile, Leu, Met, Phe, Tyr, or Trp, wherein the phenyl of X₉ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₉ is Ala, Val, Ile, Leu, Met, Phe, Tyr, or Trp.

In some embodiments, X₉ is Ala. In some embodiments, X₉ is Val. In some embodiments, X₉ is Ile. In some embodiments, X₉ is Leu. In some embodiments, X₉ is Met.

In some embodiments, X₉ is Phe, wherein the phenyl of X₉ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₉ is Phe.

In some embodiments, X₉ is Tyr, wherein the phenyl of X₉ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₉ is Tyr.

In some embodiments, X₉ is Trp, wherein the phenyl of X₉ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₉ is Trp.

In some embodiments, X₁₀ is Ala, Gly, or a Gly derivative.

In some embodiments, X₁₀ is Ala.

In some embodiments, X₁₀ is Gly or a Gly derivative.

In some embodiments, X₁₀ is Gly.

In some embodiments, X₁₀ is a Gly derivative.

In some embodiments, X₁₀ is Phg, Phg(4-OH), N-Phenethyl-Gly, N(naphtha-2-yl-ethyl)-Gly, N-(4-OMe-Phenethyl)-Gly, or Gly.

In some embodiments, X₁₀ is Phg, Phg(4-OH), N-Phenethyl-Gly, N(naphtha-2-yl-ethyl)-Gly, N-(4-OMe-Phenethyl)-Gly, or Gly, wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₀ is Phg or Ala.

In some embodiments, X₁₀ is Phg.

In some embodiments, X₁₀ is Phg, wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₀ is Phg(4-OH).

In some embodiments, X₁₀ is Phg(4-OH), wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₀ is N-Phenethyl-Gly.

In some embodiments, X₁₀ is N-Phenethyl-Gly, wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₀ is N(naphtha-2-yl-ethyl)-Gly.

In some embodiments, X₁₀ is N(naphtha-2-yl-ethyl)-Gly, wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₀ is N-(4-OMe-Phenethyl)-Gly.

In some embodiments, X₁₀ is N-(4-OMe-Phenethyl)-Gly, wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₀ is Gly.

In some embodiments, X₁₁ is Gly or a Gly derivative.

In some embodiments, X₁₁ is Gly.

In some embodiments, X₁₁ is a Gly derivative.

In some embodiments, X₁₁ is Phg, Phg(4-OH), N-Phenethyl-Gly, N(naphtha-2-yl-ethyl)-Gly, N-(4-OMe-Phenethyl)-Gly, or Gly.

In some embodiments, X₁₁ is Phg, Phg(4-OH), N-Phenethyl-Gly, N(naphtha-2-yl-ethyl)-Gly, N-(4-OMe-Phenethyl)-Gly, or Gly, wherein the phenyl of X₁₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₁ is Phg.

In some embodiments, X₁₁ is Phg, wherein the phenyl of X₁₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₁ is Phg(4-OH).

In some embodiments, X₁₁ is Phg(4-OH), wherein the phenyl of X₁₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₁ is N-Phenethyl-Gly.

In some embodiments, X₁₁ is N-Phenethyl-Gly, wherein the phenyl of X₁₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₁ is N(naphtha-2-yl-ethyl)-Gly.

In some embodiments, X₁₁ is N(naphtha-2-yl-ethyl)-Gly, wherein the phenyl of X₁₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₁ is N-(4-OMe-Phenethyl)-Gly.

In some embodiments, X₁₁ is N-(4-OMe-Phenethyl)-Gly, wherein the phenyl of X₁₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₁ is Gly.

In some embodiments, X₁₂ is an amino acid comprising a hydrophobic side chain, or a derivative thereof.

In some embodiments, X₁₂ is an amino acid comprising a hydrophobic side chain.

In some embodiments, X₁₂ is Ile, Ala, Val, Leu, Met, Phe, Tyr, or Trp.

In some embodiments, X₁₂ is Ile, Ala, Val, Leu, Met, Phe, Tyr, or Trp, wherein the phenyl of X₁₂ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₂ is Ile. In some embodiments, X₁₂ is Ala. In some embodiments, X₁₂ is Val. In some embodiments, X₁₂ is Leu. In some embodiments, X₁₂ is Met.

In some embodiments, X₁₂ is Phe.

In some embodiments, X₁₂ is Phe, wherein the phenyl of X₁₂ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₂ is Tyr.

In some embodiments, X₁₂ is Tyr, wherein the phenyl of X₁₂ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₂ is Trp.

In some embodiments, X₁₂ is Trp, wherein the phenyl of X₁₂ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₃ is an amino acid comprising a hydrophobic side chain, or a derivative thereof.

In some embodiments, X₁₃ is an amino acid comprising a hydrophobic side chain.

In some embodiments, X₁₃ is N-Me-Leu, Leu, Ala, Val, Ile, Met, Phe, Tyr, or Trp.

In some embodiments, X₁₃ is N-Me-Leu, Leu, Ala, Val, Ile, Met, Phe, Tyr, or Trp, wherein the phenyl of X₁₃ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₃ is N-Me-Leu.

In some embodiments, X₁₃ is Leu. In some embodiments, X₁₃ is Ala. In some embodiments, X₁₃ is Val. In some embodiments, X₁₃ is Ile. In some embodiments, X₁₃ is Met.

In some embodiments, X₁₃ is Phe.

In some embodiments, X₁₃ is Phe, wherein the phenyl of X₁₃ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₃ is Tyr.

In some embodiments, X₁₃ is Tyr, wherein the phenyl of X₁₃ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₃ is Trp.

In some embodiments, X₁₃ is Trp, wherein the phenyl of X₁₃ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₄ is an amino acid comprising a polar uncharged side chain, or a derivative thereof.

In some embodiments, X₁₄ is an amino acid comprising a polar uncharged side chain.

In some embodiments, X₁₄ is Thr, Ser, Asn, or Gln.

In some embodiments, X₁₄ is Thr. In some embodiments, X₁₄ is Ser. In some embodiments, X₁₄ is Asn. In some embodiments, X₁₄ is Gln.

In some embodiments, X₁₅ is 2-AOC, 2-AHP, NLE, NVA, Phe or Phe(3-Br), wherein the phenyl of X₁₅ is optionally substituted with halo, —OH, —O(C₁-C₆alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₅ is 2-AOC, 2-AHP, NLE, NVA, Phe or Phe(3-Br).

In some embodiments, X₁₅ is 2-AOC, 2-AHP, NLE, NVA, Phe or Phe(3-Br), wherein the phenyl of X₁₅ is substituted with halo, —OH, —O(C₁-C₆alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₅ is 2-AOC or NLE.

In some embodiments, X₁₅ is 2-AOC. In some embodiments, X₁₅ is 2-AHP. In some embodiments, X₁₅ is NLE. In some embodiments, X₁₅ is NVA.

In some embodiments, X₁₅ is Phe, wherein the phenyl of X₁₂ is optionally substituted with halo, —OH, —O(C₁-C₆alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₅ is Phe.

In some embodiments, X₁₅ is Phe(3-Br), wherein the phenyl of X₁₂ is optionally substituted with halo, —OH, —O(C₁-C₆alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, X₁₅ is Phe(3-Br).

In some embodiments, the capping group is selected from:

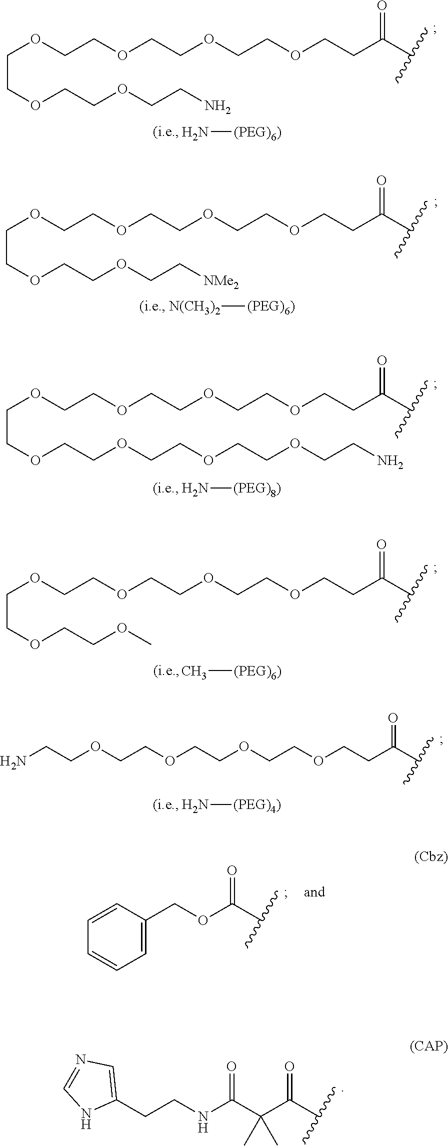

In some embodiments, the capping group is

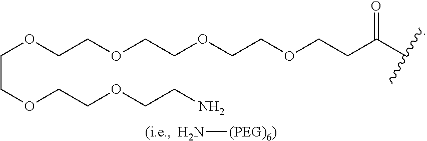

In some embodiments, the capping group is

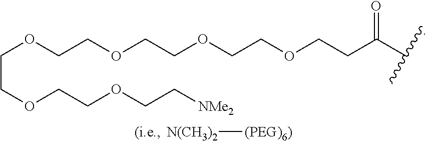

In some embodiments, the capping group is

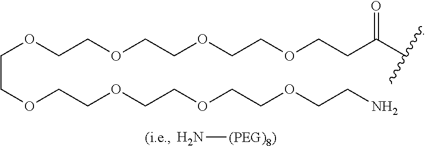

In some embodiments, the capping group is

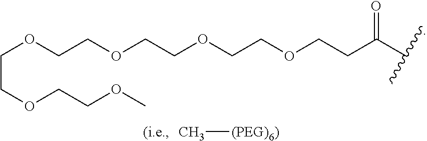

In some embodiments, the capping group is

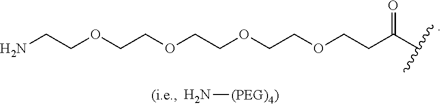

In some embodiments, the capping group is

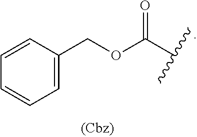 [SMILES: *C(=O)OCc1ccccc1]

In some embodiments, the capping group is

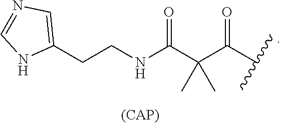 [SMILES: *C(=O)C(C)(C)C(=O)NCCc1cnc[nH]1]

In some embodiments, the peptide is of Construct Number 4 or SEQ ID NO: 5:

H₂N-(PEG)₆-X₁X₂X₃X₄X₅X₆X₇X₈X₉X₁₀X₁₁X₁₃
X₁₄-(2-AOC)-NH₂
(Construct Number 4),
(SEQ ID NO: 5)
Z₁X₁X₂X₃X₄X₅X₆X₇-Hyp(4-OH)-ALA-Phg-
GLY-ILE-LEU-THR-X₁₅-NH₂,

or an isomer, pharmaceutically acceptable salt, or prodrug thereof.

In some embodiments, the peptide is of SEQ ID NO: 6:

(SEQ ID NO: 6)
H₂N-(PEG)₆-X₁X₂X₃X₄X₅X₆X₇-Hyp(4-OH)-ALA-
Phg-GLY-ILE-LEU-THR-(2-AOC)-NH₂,

or an isomer, pharmaceutically acceptable salt, or prodrug thereof.

In some embodiments, the peptide is selected from the peptides described in Table 1 and pharmaceutically acceptable salts thereof.

In some embodiments, the peptide is selected from the peptides described in Table 1.

TABLE 1

| Peptide | Structure | | | | | | | | | | | | | | | | |
|---|---|---|---|---|---|---|---|---|---|---|---|---|---|---|---|---|---|
| No. | Z₁ | X₁ | X₂ | X₃ | X₄ | X₅ | X₆ | X₇ | X₈ | X₉ | X₁₀ | X₁₁ | X₁₂ | X₁₃ | X₁₄ | X₁₅ | NH₂ |
| 1 (SEQ ID NO: 9) | 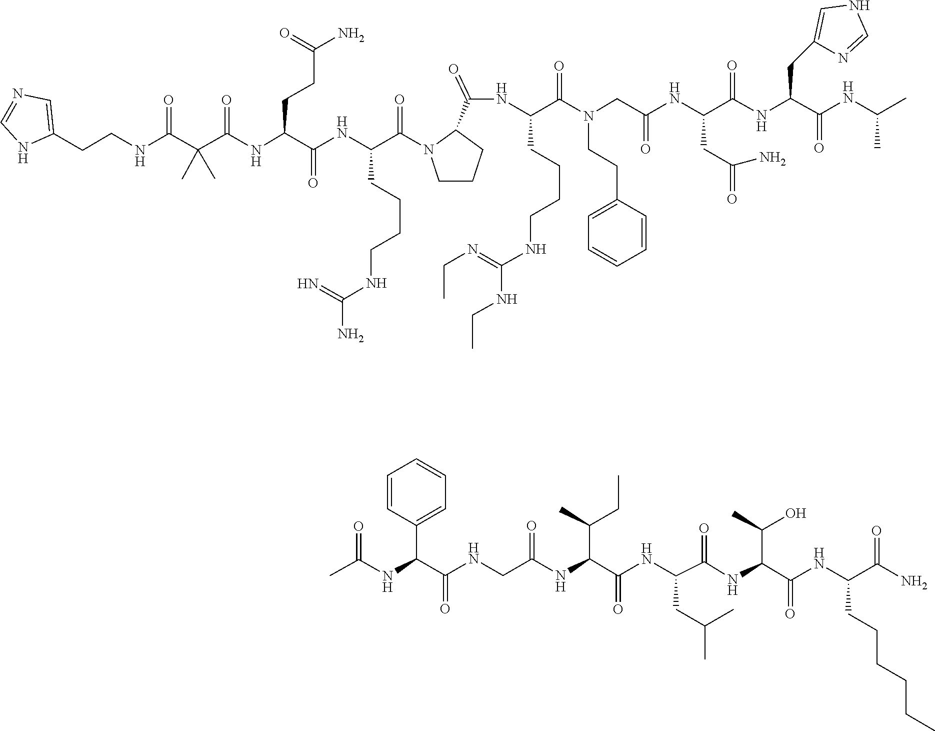 | | | | | | | | | | | | | | | | |
| | CAP | | GLN | hARG | PRO | hArg(Et)2 | N- | ASN | HIS | ALA | Phg | GLY | ILE | LEU | THR | 2- | NH2 |
| | | | | | | | Phenethyl- | | | | | | | | | AOC | |
| | | | | | | | GLY | | | | | | | | | | |
| 2 (SEQ ID NO: 10) | 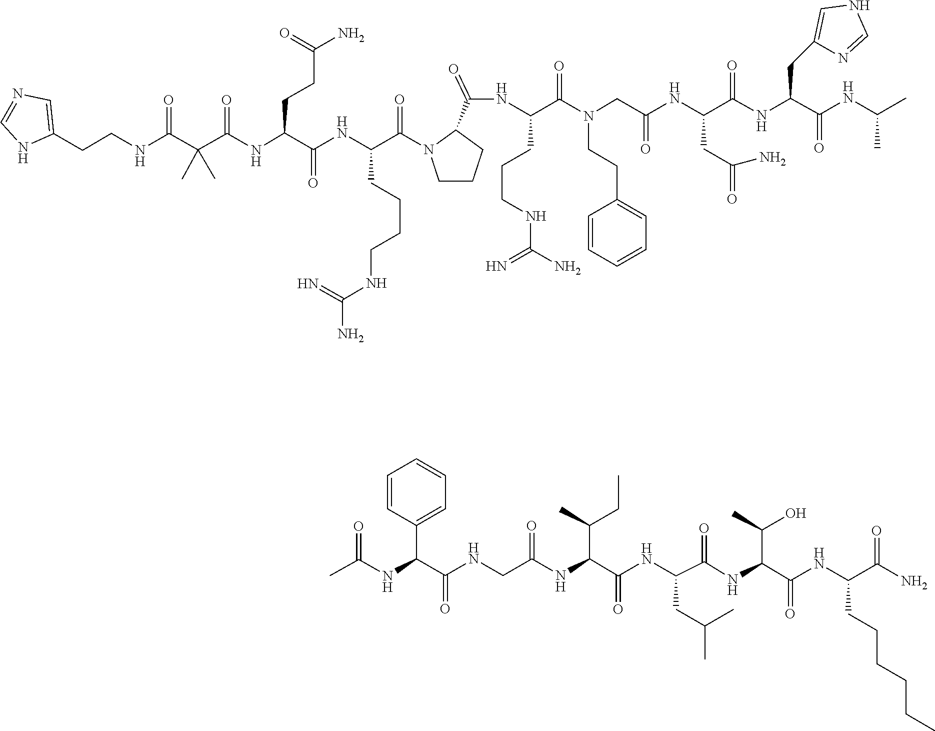 | | | | | | | | | | | | | | | | |
| | CAP | | GLN | hARG | PRO | ARG | N- | ASN | HIS | ALA | Phg | GLY | ILE | LEU | THR | 2- | NH2 |
| | | | | | | | Phenethyl- | | | | | | | | | AOC | |
| | | | | | | | GLY | | | | | | | | | | |
| 3 (SEQ ID NO: 11) | 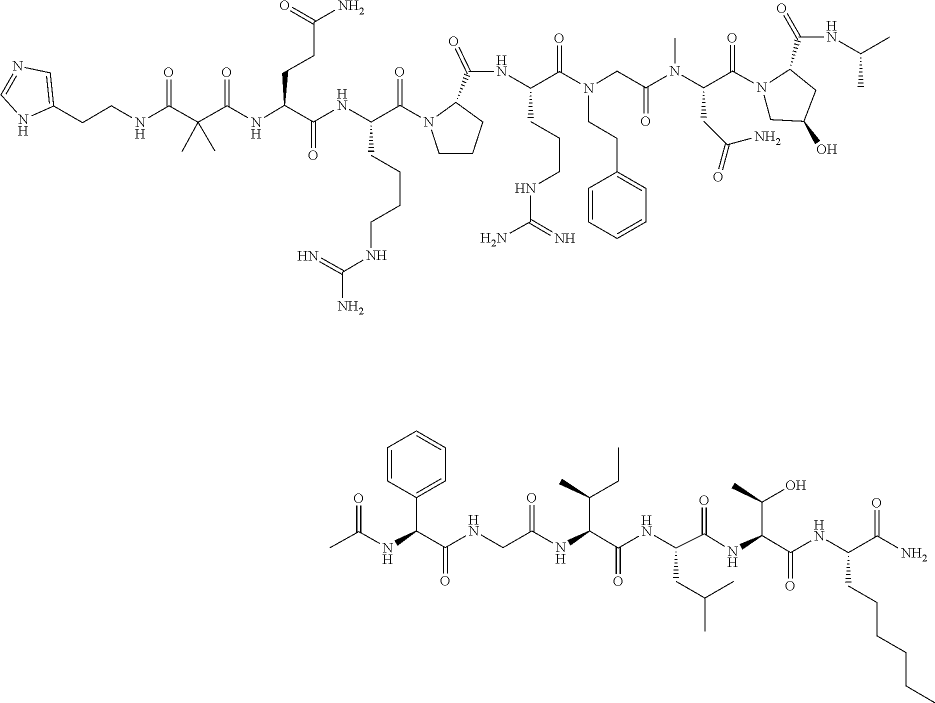 | | | | | | | | | | | | | | | | |
| | CAP | | GLN | hARG | PRO | ARG | N- | N- | Hyp(4- | ALA | Phg | GLY | ILE | LEU | THR | 2- | NH2 |
| | | | | | | | Phenethyl- | Me- | OH) | | | | | | | AOC | |
| | | | | | | | GLY | Asn | | | | | | | | | |
| 4 (SEQ ID NO: 12) | 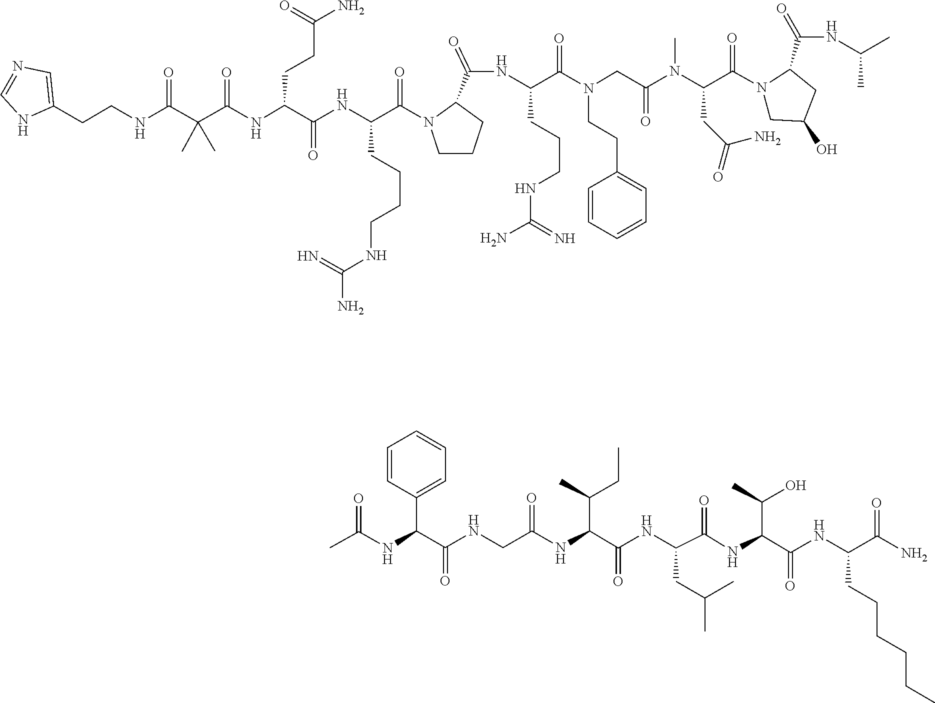 | | | | | | | | | | | | | | | | |
| | CAP | | D-GLN | hARG | PRO | ARG | N- | N- | Hyp(4- | ALA | Phg | GLY | ILE | LEU | THR | 2- | NH2 |
| | | | | | | | Phenethyl- | Me- | OH) | | | | | | | AOC | |
| | | | | | | | GLY | Asn | | | | | | | | | |
| 5 (SEQ ID NO: 13) | 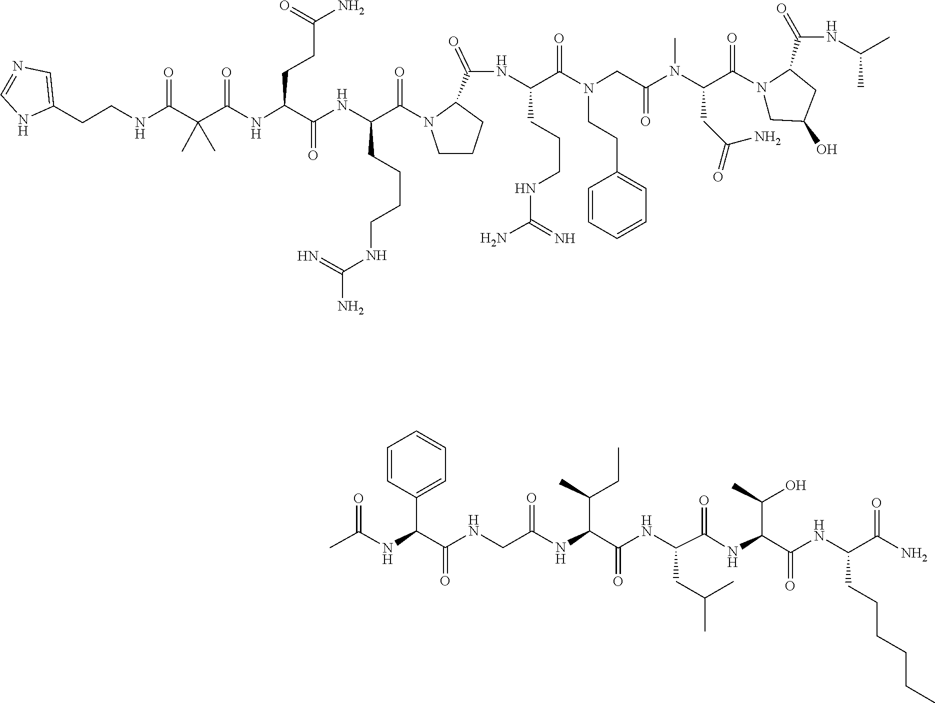 | | | | | | | | | | | | | | | | |
| | CAP | | GLN | D- | PRO | ARG | N- | N- | Hyp(4- | ALA | Phg | GLY | ILE | LEU | THR | 2- | NH2 |
| | | | | hARG | | | Phenethyl- | Me- | OH) | | | | | | | AOC | |
| | | | | | | | GLY | Asn | | | | | | | | | |
| 6 (SEQ ID NO: 14) | 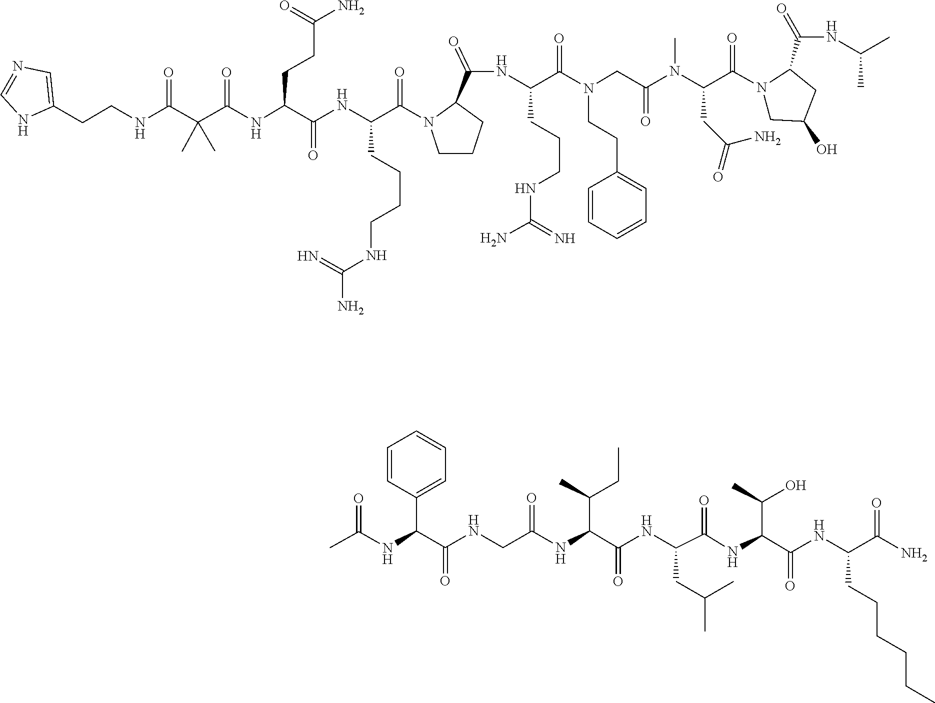 | | | | | | | | | | | | | | | | |
| | CAP | | GLN | hARG | D-PRO | ARG | N- | N- | Hyp(4- | ALA | Phg | GLY | ILE | LEU | THR | 2- | NH2 |
| | | | | | | | Phenethyl- | Me- | OH) | | | | | | | AOC | |
| | | | | | | | GLY | Asn | | | | | | | | | |
| 7 (SEQ ID NO: 15) | 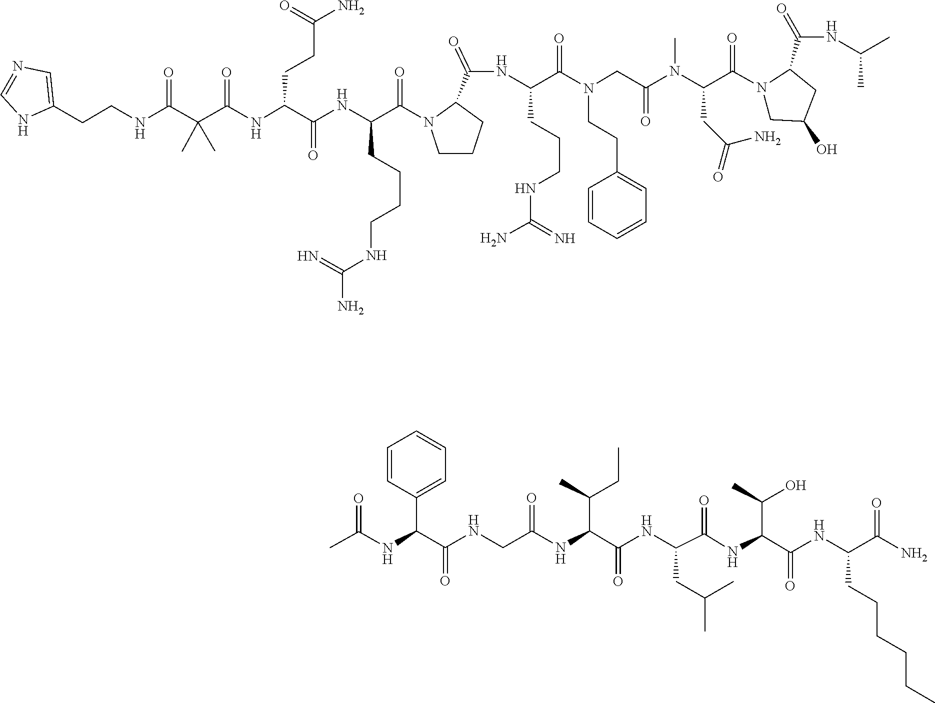 | | | | | | | | | | | | | | | | |
| | CAP | | D-GLN | D- | PRO | ARG | N- | N- | Hyp(4- | ALA | Phg | GLY | ILE | LEU | THR | 2- | NH2 |
| | | | | hARG | | | Phenethyl- | Me- | OH) | | | | | | | AOC | |
| | | | | | | | GLY | Asn | | | | | | | | | |
| 8 (SEQ ID NO: 16) | 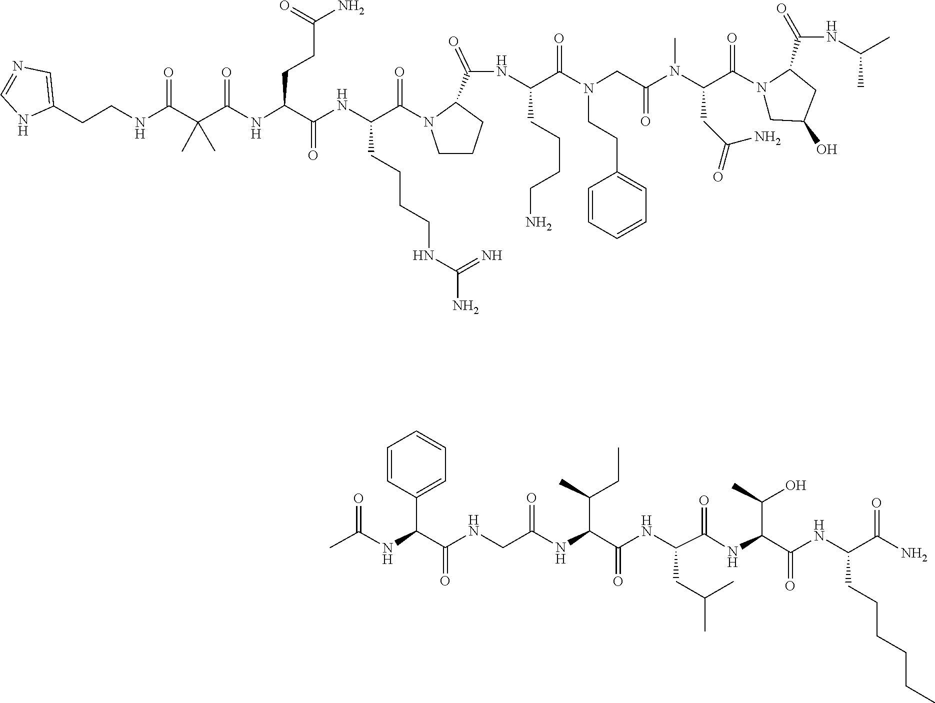 | | | | | | | | | | | | | | | | |
| | CAP | | GLN | hARG | PRO | LYS | N- | N- | Hyp(4- | ALA | Phg | GLY | ILE | LEU | THR | 2- | NH2 |
| | | | | | | | Phenethyl- | Me- | OH) | | | | | | | AOC | |
| | | | | | | | GLY | Asn | | | | | | | | | |
| 9 (SEQ ID NO: 17) | 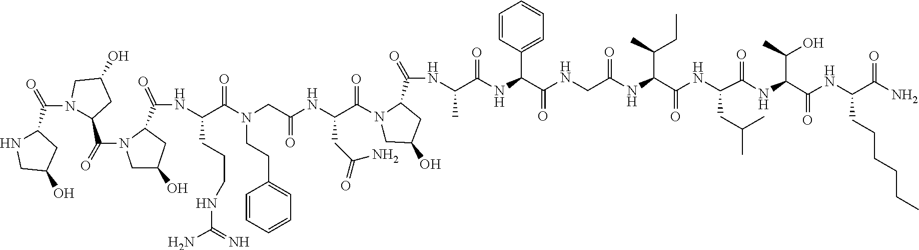 | | | | | | | | | | | | | | | | |
| | | | Hyp(4- | Hyp(4- | Hyp(4- | ARG | N- | ASN | Hyp(4- | ALA | Phg | GLY | ILE | LEU | THR | 2- | NH2 |
| | | | OH) | OH) | OH) | | Phenethyl- | | OH) | | | | | | | AOC | |
| | | | | | | | GLY | | | | | | | | | | |
| 10 (SEQ ID NO: 18) | 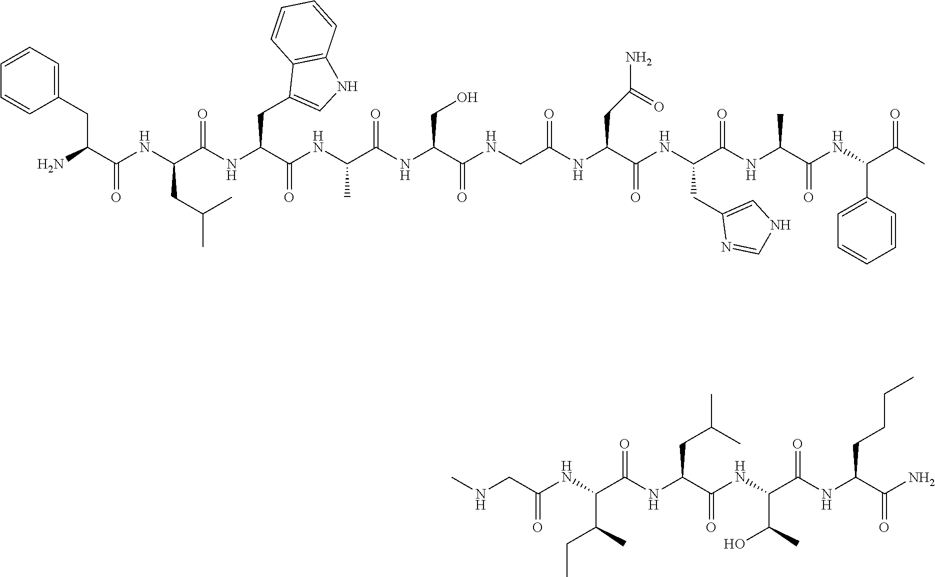 | | | | | | | | | | | | | | | | |
| | | PHE | D-LEU | TRP | ALA | SER | GLY | ASN | HIS | ALA | Phg | GLY | ILE | LEU | THR | NLE | NH2 |
| 11 (SEQ ID NO: 19) | 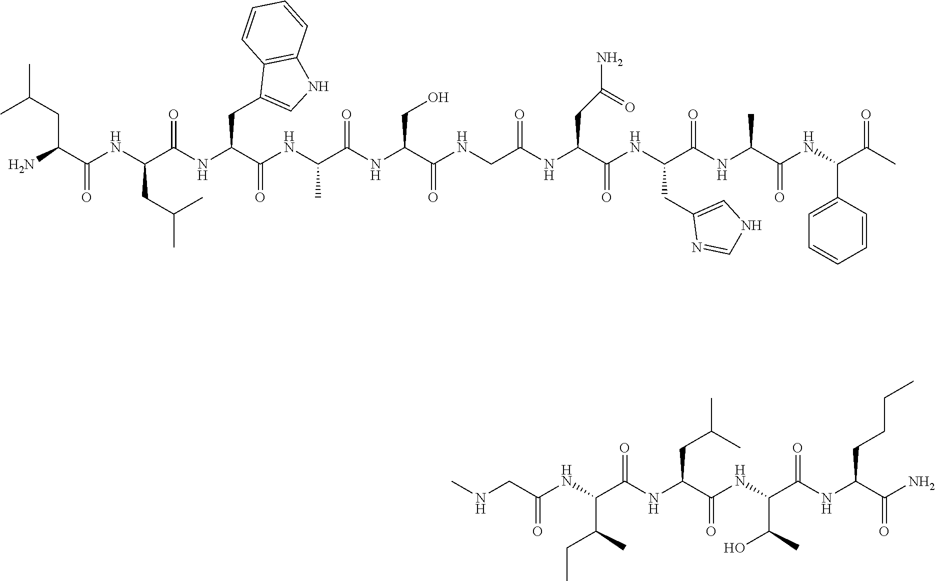 | | | | | | | | | | | | | | | | |
| | | LEU | D-LEU | TRP | ALA | SER | GLY | ASN | HIS | ALA | Phg | GLY | ILE | LEU | THR | NLE | NH2 |
| 12 (SEQ ID NO: 20) | 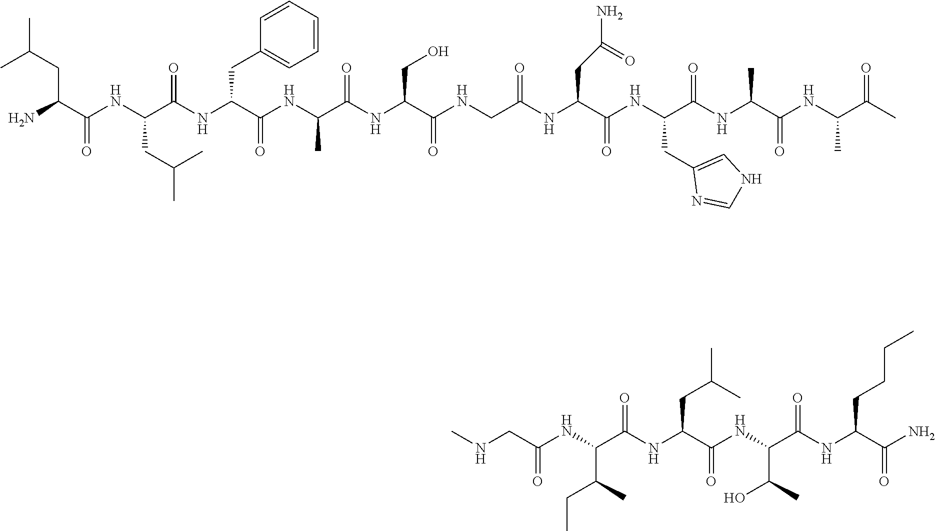 | | | | | | | | | | | | | | | | |
| | | LEU | LEU | D-PHE | D-ALA | SER | GLY | ASN | HIS | ALA | ALA | GLY | ILE | LEU | THR | NLE | NH2 |
| 13 (SEQ ID NO: 21) | 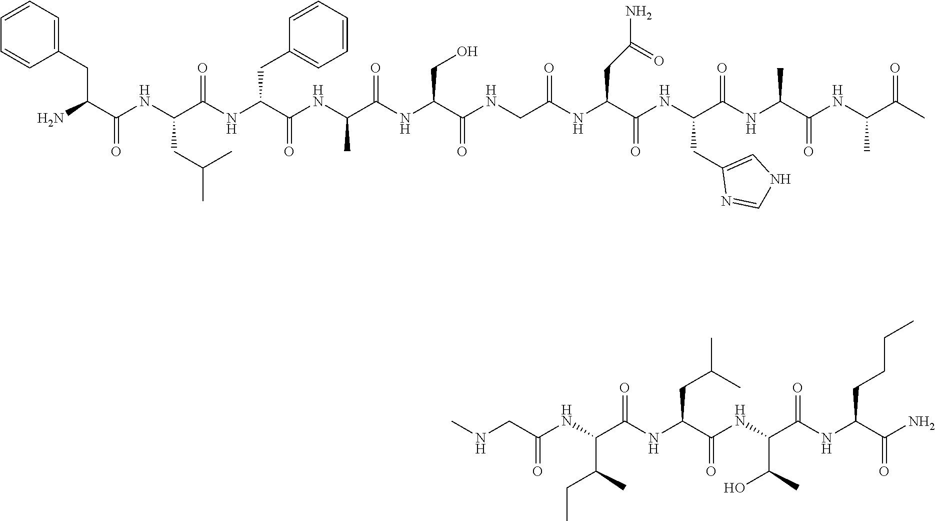 | | | | | | | | | | | | | | | | |
| | | PHE | LEU | D-PHE | D-ALA | SER | GLY | ASN | HIS | ALA | ALA | GLY | ILE | LEU | THR | NLE | NH2 |
| 14 (SEQ ID NO: 22) | 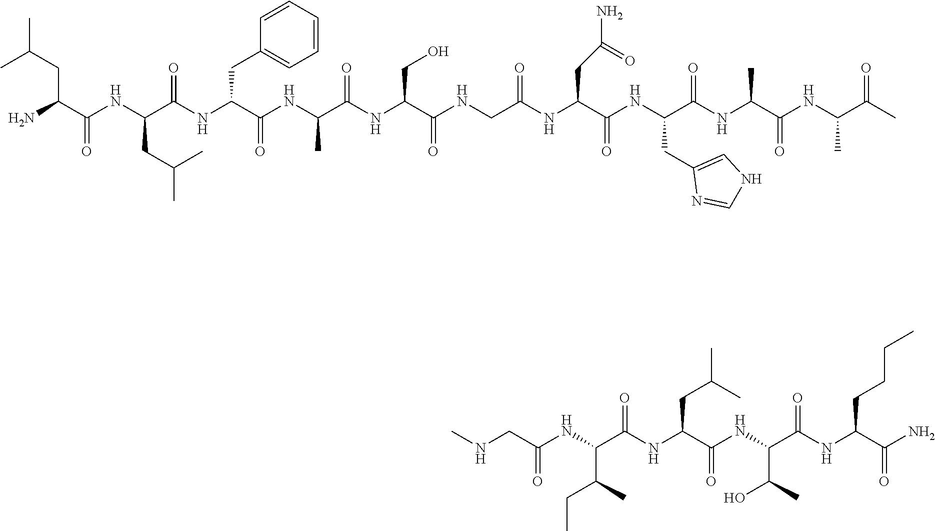 | | | | | | | | | | | | | | | | |
| | | LEU | D-LEU | D-PHE | D-ALA | SER | GLY | ASN | HIS | ALA | ALA | GLY | ILE | LEU | THR | NLE | NH2 |
| 15 (SEQ ID NO: 23) | 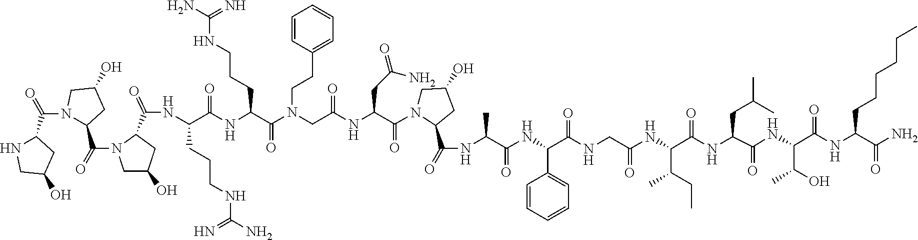 | | | | | | | | | | | | | | | | |
| | | Hyp(4- | Hyp(4- | Hyp(4- | ARG | ARG | N- | ASN | Hyp(4- | ALA | Phg | GLY | ILE | LEU | THR | 2- | NH2 |
| | | OH) | OH) | OH) | | | Phenethyl- | | OH) | | | | | | | AOC | |
| | | | | | | | GLY | | | | | | | | | | |
| 16 (SEQ ID NO: 24) | 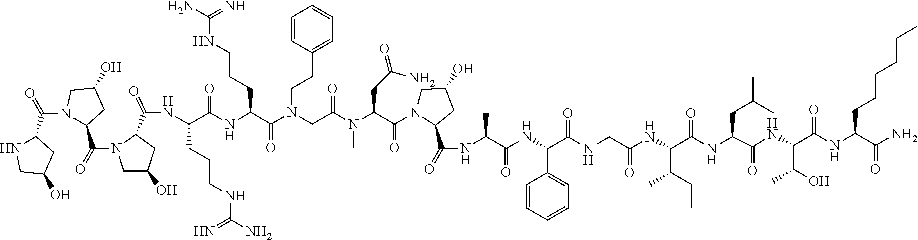 | | | | | | | | | | | | | | | | |
| | | Hyp(4- | Hyp(4- | Hyp(4- | ARG | ARG | N- | N- | Hyp(4- | ALA | Phg | GLY | ILE | LEU | THR | 2- | NH2 |
| | | OH) | OH) | OH) | | | Phenethyl- | Me- | OH) | | | | | | | AOC | |
| | | | | | | | GLY | Asn | | | | | | | | | |
| 17 (SEQ ID NO: 25) | 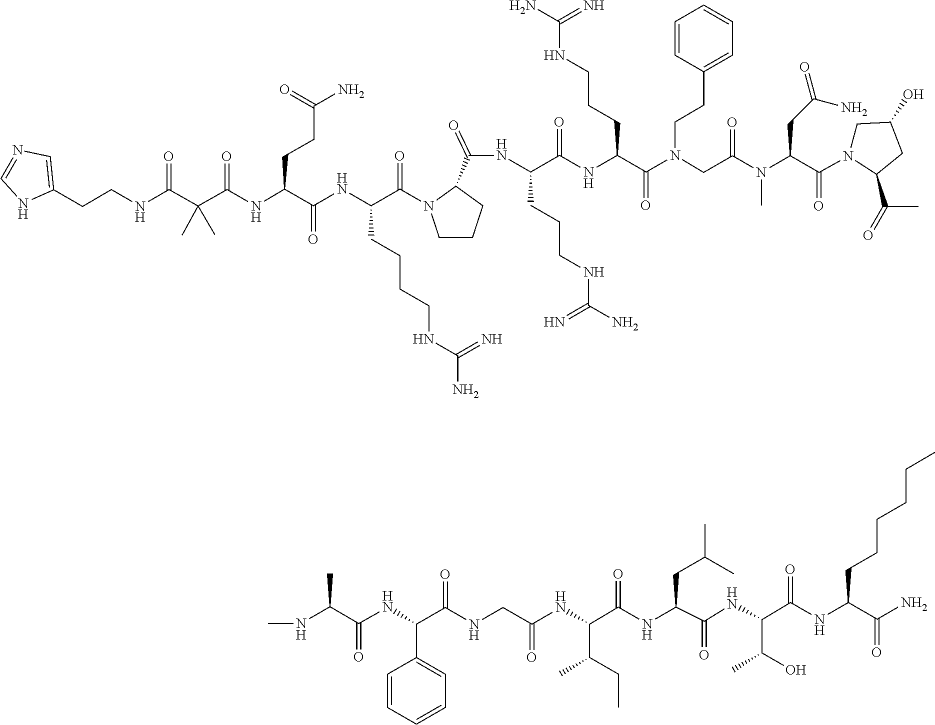 | | | | | | | | | | | | | | | | |
| | CAP | GLN | hARG | PRO | ARG | ARG | N- | N- | Hyp(4- | ALA | Phg | GLY | ILE | LEU | THR | 2- | NH2 |
| | | | | | | | Phenethyl- | Me- | OH) | | | | | | | AOC | |
| | | | | | | | GLY | Asn | | | | | | | | | |
| 18 (SEQ ID NO: 26) | 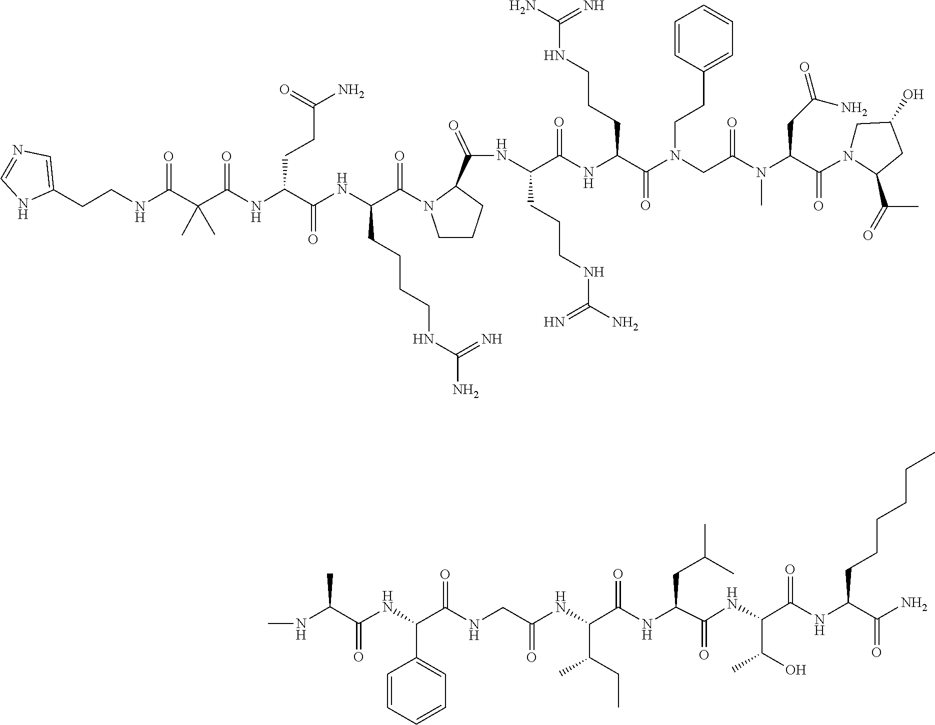 | | | | | | | | | | | | | | | | |
| | CAP | D-GLN | D- | D-PRO | ARG | ARG | N- | N- | Hyp(4- | ALA | Phg | GLY | ILE | LEU | THR | 2- | NH2 |
| | | | hARG | | | | Phenethyl- | Me- | OH) | | | | | | | AOC | |
| | | | | | | | GLY | Asn | | | | | | | | | |
| 19 (SEQ ID NO: 27) | 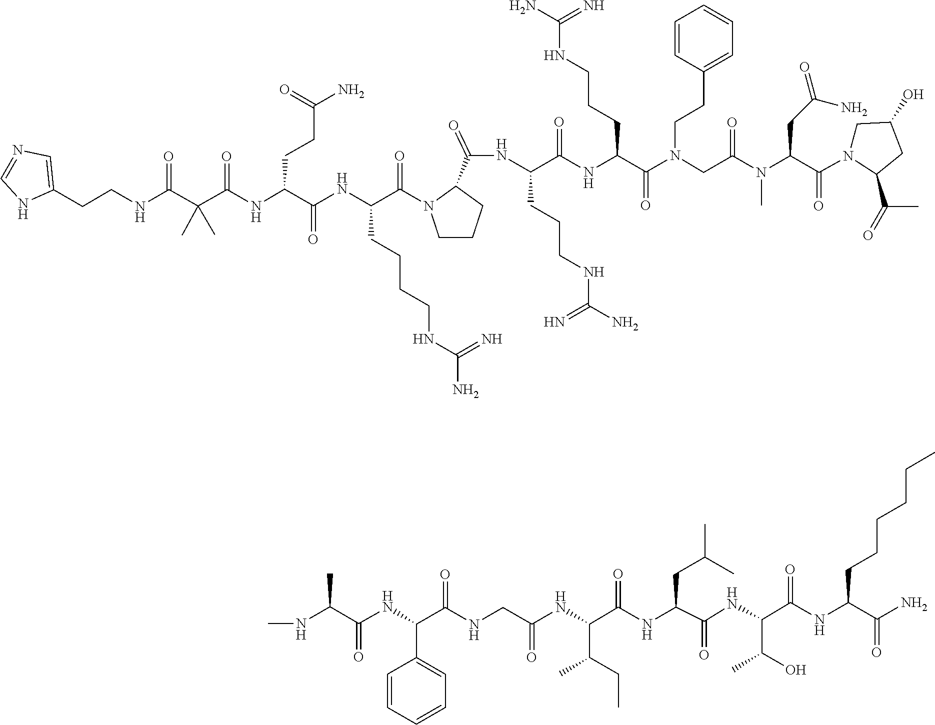 | | | | | | | | | | | | | | | | |
| | CAP | D-GLN | hARG | PRO | ARG | ARG | N- | N- | Hyp(4- | ALA | Phg | GLY | ILE | LEU | THR | 2- | NH2 |
| | | | | | | | Phenethyl- | Me- | OH) | | | | | | | AOC | |
| | | | | | | | GLY | Asn | | | | | | | | | |
| 20 (SEQ ID NO: 28) | 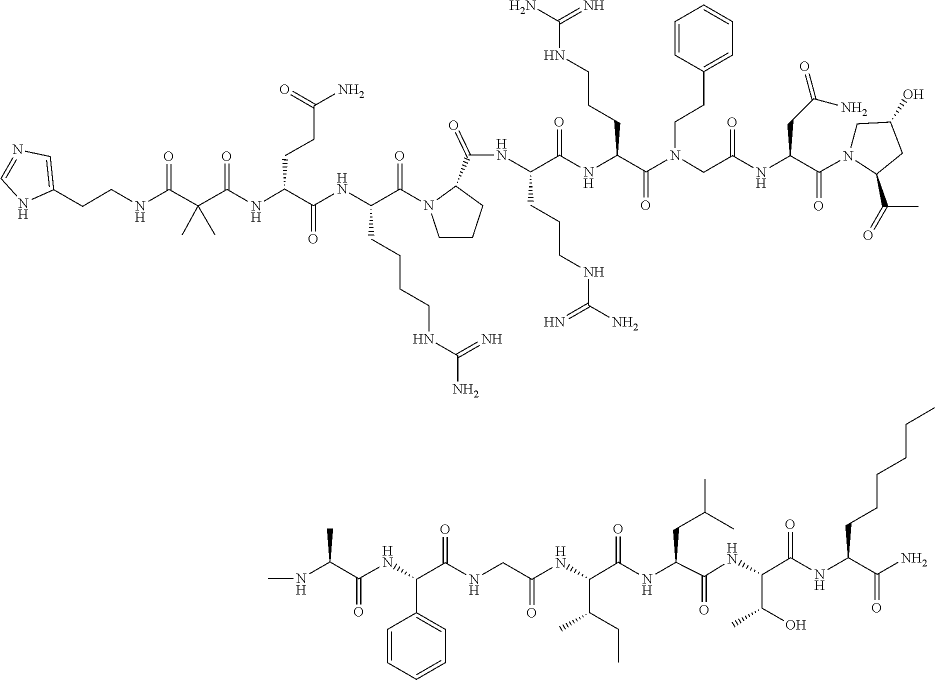 | | | | | | | | | | | | | | | | |
| | CAP | D-GLN | hARG | PRO | ARG | ARG | N- | ASN | Hyp(4- | ALA | Phg | GLY | ILE | LEU | THR | 2- | NH2 |
| | | | | | | | Phenethyl- | | OH) | | | | | | | AOC | |
| | | | | | | | GLY | | | | | | | | | | |

In some embodiments, the peptide is a pharmaceutically acceptable salt of any one of the peptides described in Table 1.

In some aspects, the present disclosure provides a peptide being an isotopic derivative (e.g., isotopically labeled peptide) of any one of the peptides of the Formulae disclosed herein.

In some embodiments, the peptide is an isotopic derivative of any one of the peptides described in Table 1 and prodrugs and pharmaceutically acceptable salts thereof.

In some embodiments, the peptide is an isotopic derivative of any one of the peptides described in Table 1 and pharmaceutically acceptable salts thereof.

In some embodiments, the peptide is an isotopic derivative of any one of prodrugs of the peptides described in Table 1 and pharmaceutically acceptable salts thereof.

In some embodiments, the peptide is an isotopic derivative of any one of the peptide described in Table 1.

It is understood that the isotopic derivative can be prepared using any of a variety of art-recognised techniques. For example, the isotopic derivative can generally be prepared by carrying out the procedures disclosed in the Examples described herein, by substituting an isotopically labeled reagent for a non-isotopically labeled reagent.

In some embodiments, the isotopic derivative is a deuterium labeled peptide.

In some embodiments, the isotopic derivative is a deuterium labeled peptide of any one of the peptides of the Formulae disclosed herein.

The term “isotopic derivative”, as used herein, refers to a derivative of a peptide in which one or more atoms are isotopically enriched or labelled. For example, an isotopic derivative of a peptide of Construct Number 1, Construct Number 2, or Construct Number 3 is isotopically enriched with regard to, or labelled with, one or more isotopes as compared to the corresponding peptide of Construct Number 1, Construct Number 2, or Construct Number 3. In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled peptide (i.e., being enriched with ²H with regard to one or more atoms thereof). In some embodiments, the peptide is a ¹⁸F labeled peptide. In some embodiments, the peptide is a ¹²³I labeled peptide, a ¹²⁴I labeled peptide, a ¹²⁵I labeled peptide, a ¹²⁹I labeled peptide, a ¹³¹I labeled peptide, a ¹³⁵I labeled peptide, or any combination thereof. In some embodiments, the peptide is a ³³S labeled peptide, a ³⁴S labeled peptide, a ³⁵S labeled peptide, a ³⁶S labeled peptide, or any combination thereof.

It is understood that the ¹⁸F, ¹²³I, ¹²⁴I, ¹²⁵I, ¹²⁹I, ¹³¹I, ¹³⁵I, ³²S, ³⁴S, ³⁵S, and/or ³⁶S labeled peptide, can be prepared using any of a variety of art-recognised techniques. For example, the deuterium labeled peptide can generally be prepared by carrying out the procedures disclosed in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A peptide of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S, ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

The various functional groups and substituents making up the peptides of the Construct Number 1, Construct Number 2, or Construct Number 3 are typically chosen such that the molecular weight of the peptide does not exceed 1000 daltons. More usually, the molecular weight of the peptide will be less than 900, for example less than 800, or less than 750, or less than 700, or less than 650 daltons. More conveniently, the molecular weight is less than 600 and, for example, is 550 daltons or less.

A suitable pharmaceutically acceptable salt of a peptide of the disclosure is, for example, an acid-addition salt of a peptide of the disclosure which is sufficiently basic, for example, an acid-addition salt with, for example, an inorganic organic acid, for example hydrochloric, hydrobromic, sulfuric, phosphoric, trifluoroacetic, formic, citric methane sulfonate or maleic acid. In addition, a suitable pharmaceutically acceptable salt of a peptide of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the peptides of any one of the sequences disclosed herein and any pharmaceutically acceptable salts thereof, comprise stereoisomers, mixtures of stereoisomers, polymorphs of all isomeric forms of said peptides.

It will be understood that while peptides disclosed herein may be presented in one particular configuration. Such particular configuration is not to be construed as limiting the disclosure to one or another isomer, tautomer, regioisomer or stereoisomer, nor does it exclude mixtures of isomers, tautomers, regioisomers or stereoisomers. In some embodiments, the presentation of a peptide herein in a particular configuration intends to encompass, and to refer to, each of the available isomers, tautomers, regioisomers, and stereoisomers of the peptide, or any mixture thereof, while the presentation further intends to refer to the specific configuration of the peptide.

It will be understood that while peptides disclosed herein may be presented without specified configuration (e.g., without specified stereochemistry). Such presentation intends to encompass all available isomers, tautomers, regioisomers, and stereoisomers of the peptide. In some embodiments, the presentation of a peptide herein without specified configuration intends to refer to each of the available isomers, tautomers, regioisomers, and stereoisomers of the peptide, or any mixture thereof.

As used herein, the term “isomerism” means peptides that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral centre” refers to a carbon atom bonded to four nonidentical substituents.

As used herein, the term “chiral isomer” means a peptide with at least one chiral centre. Peptides with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the peptides of the present disclosure may be depicted as different chiral isomers or geometric isomers. It is also to be understood that when peptides have chiral isomeric or geometric isomeric forms, all isomeric forms are intended to be included in the scope of the present disclosure, and the naming of the peptides does not exclude any isomeric forms, it being understood that not all isomers may have the same level of activity.

It is to be understood that the structures and other peptides discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the peptides of the present disclosure may be depicted as different tautomers. It should also be understood that when peptides have tautomeric forms, all tautomeric forms are intended to be included in the scope of the present disclosure, and the naming of the peptides does not exclude any tautomer form. It will be understood that certain tautomers may have a higher level of activity than others.

Peptides that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a peptide has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral peptide can exist as either individual enantiomer or as a mixture thereof. A mixture containing equal proportions of the enantiomers is called a “racemic mixture”.

The peptides of this disclosure may possess one or more asymmetric centres; such peptides can therefore be produced as individual (R)- or (S)-stereoisomers or as mixtures thereof. Unless indicated otherwise, the description or naming of a particular peptide in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the peptides of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses peptides of the disclosure as defined herein which comprise one or more isotopic substitutions.

It is to be understood that the peptides of any sequences described herein include the peptides themselves, as well as their salts, and their solvates, if applicable. A salt, for example, can be formed between an anion and a positively charged group (e.g., amino) on a substituted peptide disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted peptide disclosed herein. Suitable cations include sodium ion, potassium ion, magnesium ion, calcium ion, and an ammonium cation such as tetramethylammonium ion or diethylamine ion. The substituted peptides disclosed herein also include those salts containing quaternary nitrogen atoms.

It is to be understood that the peptides of the present disclosure, for example, the salts of the peptides, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some peptides have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical peptide that is structurally similar to another but differs slightly in composition (as in the replacement of one atom by an atom of a different element or in the presence of a particular functional group, or the replacement of one functional group by another functional group). Thus, an analog is a peptide that is similar or comparable in function and appearance, but not in structure origin to the reference peptide.

As used herein, the term “derivative” refers to peptides that have a common core structure and are substituted with various groups as described herein.

As used herein, the term “bioisostere” refers to a peptide resulting from the exchange of an atom or of a group of atoms with another, broadly similar, atom or group of atoms. The objective of a bioisosteric replacement is to create a new peptide with similar biological properties to the parent peptide. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain peptides of any one of the sequences disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain peptides of any one of the sequences disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NTR) spectroscopy, solution and/or solid state nuclear magnetic resonance spectroscopy. The water content of such crystalline materials may be determined by Karl Fischer analysis.

Peptides of any one of the sequences disclosed herein may exist in a number of different tautomeric forms and references to peptides of Construct Number 1, Construct Number 2, or Construct Number 3 include all such forms. For the avoidance of doubt, where a peptide can exist in one of several tautomeric forms, and only one is specifically described or shown, all others are nevertheless embraced by Construct Number 1, Construct Number 2, or Construct Number 3. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

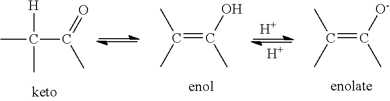

Peptides of any one of the sequences disclosed herein containing an amine function may also form N-oxides. A reference herein to a peptide of Construct Number 1, Construct Number 2, or Construct Number 2 that contains an amine function also includes the N-oxide. Where a peptide contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine peptide is reacted with meta-chloroperoxybenzoic acid (mCPBA), for example, in an inert solvent such as dichloromethane.

The peptides of any one of the sequences disclosed herein may be administered in the form of a prodrug which is broken down in the human or animal body to release a peptide of the disclosure. A prodrug may be used to alter the physical properties and/or the pharmacokinetic properties of a peptide of the disclosure. A prodrug can be formed when the peptide of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those peptides of any one of the sequences disclosed herein as defined hereinbefore when made available by organic synthesis and when made available within the human or animal body by way of cleavage of a prodrug thereof. Accordingly, the present disclosure includes those peptides of any one of the sequences disclosed herein that are produced by organic synthetic means and also such peptides that are produced in the human or animal body by way of metabolism of a precursor peptide, that is a peptide of any one of the sequences disclosed herein may be a synthetically-produced peptide or a metabolically-produced peptide.

A suitable pharmaceutically acceptable prodrug of a peptide of any one of the sequences disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a peptide of any one of the sequences disclosed herein that possesses a hydroxy group is, for example, an in vivo cleavable ester or ether thereof. An in vivo cleavable ester or ether of a peptide of any one of the sequences disclosed herein containing a hydroxy group is, for example, a pharmaceutically acceptable ester or ether which is cleaved in the human or animal body to produce the parent hydroxy peptide. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a peptide of any one of the sequences disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a peptide of any one of the sequences disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl)piperazin-1-ylmethyl.

The in vivo effects of a peptide of any one of the sequences disclosed herein may be exerted in part by one or more metabolites that are formed within the human or animal body after administration of a peptide of any one of the sequences disclosed herein. As stated hereinbefore, the in vivo effects of a peptide of any one of the sequences disclosed herein may also be exerted by way of metabolism of a precursor peptide (a prodrug).

Suitably, the present disclosure excludes any individual peptides not possessing the biological activity defined herein.

### Methods of Synthesis

In some aspects, the present disclosure provides a method of preparing a peptide of the present disclosure.

In some aspects, the present disclosure provides a method of preparing a peptide, comprising one or more steps as described herein.

In some aspects, the present disclosure provides a peptide obtainable by, or obtained by, or directly obtained by a method for preparing a peptide as described herein.

In some aspects, the present disclosure provides an intermediate as described herein, being suitable for use in a method for preparing a peptide as described herein.

The peptides of the present disclosure can be prepared by any suitable technique known in the art. Particular processes for the preparation of these peptides are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the peptides of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a peptide of Construct Number 1, Construct Number 2, or Construct Number 3 has been synthesised by any one of the processes defined herein, the processes may then further comprise the additional steps of: (i) removing any protecting groups present; (ii) converting the peptide of Construct Number 1, Construct Number 2, or Construct Number 3 into another peptide of Construct Number 1, Construct Number 2, or Construct Number 3; (iii) forming a pharmaceutically acceptable salt, hydrate or solvate thereof, and/or (iv) forming a prodrug thereof.

The resultant peptides of Construct Number 1, Construct Number 2, or Construct Number 3 can be isolated and purified using techniques well known in the art.

Conveniently, the reaction of the peptides is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro peptides, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective peptides and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional peptides of the present disclosure can be readily prepared. Those skilled in the art will readily understand that known variations of the conditions and processes of the following preparative procedures can be used to prepare these peptides.

As will be understood by the person skilled in the art of organic synthesis, peptides of the present disclosure are readily accessible by various synthetic routes, some of which are exemplified in the accompanying examples. In some embodiments, the synthetic route is solid phase peptide synthesis. The skilled person will easily recognise which kind of reagents and reactions conditions are to be used and how they are to be applied and adapted in any particular instance—wherever necessary or useful—in order to obtain the peptides of the present disclosure. Furthermore, some of the peptides of the present disclosure can readily be synthesised by reacting other peptides of the present disclosure under suitable conditions, for instance, by converting one particular functional group being present in a peptide of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

### Biological Assays

Peptides designed, selected and/or optimised by methods described above, once produced, can be characterised using a variety of assays known to those skilled in the art to determine whether the peptides have biological activity. For example, the molecules can be characterised by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the peptides of the present disclosure. These in vitro or in vivo biological assays can include, but are not limited to, enzymatic activity assays, electrophoretic mobility shift assays, reporter gene assays, in vitro cell viability assays, and the assays described herein.

Despite orexin cell loss and decreased orexin peptides in cerebrospinal fluid in NT1, orexin receptors on post synaptic neurons remain intact as suitable targets for pharmacotherapeutic intervention. The orexin peptides A and B (OXA and OXB) may be cleaved from a single precursor molecule (prepro-orexin) that is produced exclusively in the lateral hypothalamus. Both orexin peptides bind with similar high affinity to OX2R, but the orexin-1 receptor (OX1R) may be preferentially bound by OXA. Postsynaptic excitation of these G-protein coupled orexin receptors may stimulate the release of monoaminergic and cholinergic neurotransmitters that promote wakefulness and inhibitory neurotransmitters that suppress REM sleep atonia.

In some embodiments, the assay uses cells stably expressing either human orexin type 2 or human orexin type 1 receptor. In some embodiments, the cells are obtained by inserting the Orexin receptor cDNA into pcDNA3.1(+) plasmid vector, identifying clones by G418 drug resistance selection, and/or growing a single clone for OX2R-CHO and OX1R-CHO in bulk. In some embodiments, the assay is detected using Fluorescent Imaging Plate Reader TETRA (FLIPR TETRA: manufactured by Molecular Devices). In some embodiments, the agonist activity of the test peptide is calculated assuming that the fluorescence value of the well containing only the dilution buffer is 0% and the fluorescence value of the well containing 10 nM human orexin A (Tocris) buffer is 100%.

In some embodiments, the biological assay is described in the Examples herein.

### Pharmaceutical Compositions

In some aspects, the present disclosure provides a pharmaceutical composition comprising a peptide of the present disclosure as an active ingredient. In some embodiments, the present disclosure provides a pharmaceutical composition comprising at least one peptide of each of the formulae described herein, or a pharmaceutically acceptable salt or solvate thereof, and one or more pharmaceutically acceptable carriers or excipients. In some embodiments, the present disclosure provides a pharmaceutical composition comprising at least one peptide selected from Table 1.

As used herein, the term “composition” is intended to encompass a product comprising the specified ingredients in the specified amounts, as well as any product which results, directly or indirectly, from combination of the specified ingredients in the specified amounts.

The peptides of present disclosure can be formulated for oral administration in forms such as tablets, capsules (each of which includes sustained release or timed release formulations), pills, powders, granules, elixirs, tinctures, suspensions, syrups and emulsions. The peptides of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkynyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active peptide can be incorporated with excipients and used in the form of tablets, troches, or capsules. Oral compositions can also be prepared using a fluid carrier for use as a mouthwash, wherein the peptide in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or peptides of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, orange flavoring.

According to a further aspect of the disclosure there is provided a pharmaceutical composition which comprises a peptide of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a peptide of the present disclosure for use in therapy is an amount sufficient to treat or prevent an inflammasome related condition referred to herein, slow its progression and/or reduce the symptoms associated with the condition.

An effective amount of a peptide of the present disclosure for use in therapy is an amount sufficient to treat an inflammasome related condition referred to herein, slow its progression and/or reduce the symptoms associated with the condition.

The size of the dose for therapeutic or prophylactic purposes of a peptide of Construct Number 1, Construct Number 2, or Construct Number 3 will naturally vary according to the nature and severity of the conditions, the age and sex of the animal or patient and the route of administration, according to well-known principles of medicine.

### Methods of Use

In some aspects, the present disclosure provides a method of modulating orexin receptor activity (e.g., in vitro or in vivo), comprising contacting a cell with an effective amount of a peptide of the present disclosure or an isomer, pharmaceutically acceptable salt, or prodrug thereof.

In some aspects, the present disclosure provides a method of modulating orexin-2 receptor activity (e.g., in vitro or in vivo), comprising contacting a cell with an effective amount of a peptide of the present disclosure or an isomer, pharmaceutically acceptable salt, or prodrug thereof.

In some aspects, the present disclosure provides a method of treating or preventing a disease or disorder disclosed herein in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating a disease or disorder disclosed herein in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some embodiments, the disease or disorder is associated with an implicated orexin receptor activity. In some embodiments, the disease or disorder is a disease or disorder in which orexin receptor activity is implicated.

In some embodiments, the disease or disorder is associated with an implicated orexin-2 receptor activity. In some embodiments, the disease or disorder is a disease or disorder in which orexin-2 receptor activity is implicated.

In some embodiments, the disease or disorder is narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia.

In some aspects, the present disclosure provides a method of treating or preventing narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing narcolepsy in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing a hypersomnia disorder in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing a neurodegenerative disorder in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing a symptom of a rare genetic disorder in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing a mental health disorder in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing a metabolic syndrome in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing osteoporosis in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing cardiac failure in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing coma in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating or preventing a complication in emergence from anesthesia in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating narcolepsy in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating a hypersomnia disorder in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating a neurodegenerative disorder in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating a symptom of a rare genetic disorder in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating a mental health disorder in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating a metabolic syndrome in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating osteoporosis in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating cardiac failure in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating coma in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method of treating a complication in emergence from anesthesia in a subject in need thereof, comprising administering to the subject a therapeutically effective amount of a peptide of the present disclosure or an isomer, pharmaceutically acceptable salt, or prodrug thereof, or a pharmaceutical composition of the present disclosure.

In some aspects, the present disclosure provides a method, use, or peptide or pharmaceutical composition for use of the peptide disclosed herein in combination with one or more additional therapy.

In some embodiments, the one or more additional therapy is oral birth control.

In some embodiments, the one or more additional therapy is administered for the treatment of excessive sleepiness.

In some embodiments, the one or more additional therapy is administered for the treatment of comorbid indications.

In some embodiments, the comorbid indication is depression.

In some embodiments, the comorbid indication is a psychiatric disorder.

In some embodiments, the comorbid indication is hypertension.

In some embodiments, the comorbid indication is a metabolic disorder.

In some aspects, the present disclosure provides a peptide of the present disclosure or an isomer, pharmaceutically acceptable salt, or prodrug thereof for use in modulating orexin receptor activity (e.g., in vitro or in vivo).

In some aspects, the present disclosure provides a peptide of the present disclosure or an isomer, pharmaceutically acceptable salt, or prodrug thereof for use in modulating orexin-2 receptor activity (e.g., in vitro or in vivo).

In some aspects, the present disclosure provides a peptide of the present disclosure or an isomer, pharmaceutically acceptable salt, or prodrug thereof for use in treating or preventing a disease or disorder disclosed herein.

In some aspects, the present disclosure provides a peptide of the present disclosure or an isomer, pharmaceutically acceptable salt, or prodrug thereof for use in treating a disease or disorder disclosed herein.

In some aspects, the present disclosure provides a peptide of the present disclosure or an isomer, pharmaceutically acceptable salt, or prodrug thereof for use in treating or preventing narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof.

In some aspects, the present disclosure provides a peptide of the present disclosure or an isomer, pharmaceutically acceptable salt, or prodrug thereof for use in treating or preventing narcolepsy in a subject in need thereof.

In some aspects, the present disclosure provides a peptide of the present disclosure or an isomer, pharmaceutically acceptable salt, or prodrug thereof for use in treating or preventing a hypersomnia disorder in a subject in need thereof.

In some aspects, the present disclosure provides a peptide of the present disclosure or an isomer, pharmaceutically acceptable salt, or prodrug thereof for use in treating or preventing a neurodegenerative disorder in a subject in need thereof.

In some aspects, the present disclosure provides a peptide of the present disclosure or an isomer, pharmaceutically acceptable salt, or prodrug thereof for use in treating or preventing a symptom of a rare genetic disorder in a subject in need thereof.

In some aspects, the present disclosure provides a peptide of the present disclosure or an isomer, pharmaceutically acceptable salt, or prodrug thereof for use in treating or preventing a mental health disorder in a subject in need thereof.

In some aspects, the present disclosure provides a peptide of the present disclosure or an isomer, pharmaceutically acceptable salt, or prodrug thereof for use in treating or preventing a metabolic syndrome in a subject in need thereof.

In some aspects, the present disclosure provides a peptide of the present disclosure or an isomer, pharmaceutically acceptable salt, or prodrug thereof for use in treating or preventing osteoporosis in a subject in need thereof.

In some aspects, the present disclosure provides a peptide of the present disclosure or an isomer, pharmaceutically acceptable salt, or prodrug thereof for use in treating or preventing cardiac failure in a subject in need thereof.

In some aspects, the present disclosure provides a peptide of the present disclosure or an isomer, pharmaceutically acceptable salt, or prodrug thereof for use in treating or preventing coma in a subject in need thereof.

In some aspects, the present disclosure provides a peptide of the present disclosure or an isomer, pharmaceutically acceptable salt, or prodrug thereof for use in treating or preventing a complication in emergence from anesthesia in a subject in need thereof.

In some aspects, the present disclosure provides a peptide of the present disclosure or an isomer, pharmaceutically acceptable salt, or prodrug thereof for use in treating narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof.

In some aspects, the present disclosure provides a peptide of the present disclosure or an isomer, pharmaceutically acceptable salt, or prodrug thereof for use in treating narcolepsy in a subject in need thereof.

In some aspects, the present disclosure provides a peptide of the present disclosure or an isomer, pharmaceutically acceptable salt, or prodrug thereof for use in treating a hypersomnia disorder in a subject in need thereof.

In some aspects, the present disclosure provides a peptide of the present disclosure or an isomer, pharmaceutically acceptable salt, or prodrug thereof for use in treating a neurodegenerative disorder in a subject in need thereof.

In some aspects, the present disclosure provides a peptide of the present disclosure or an isomer, pharmaceutically acceptable salt, or prodrug thereof for use in treating a symptom of a rare genetic disorder in a subject in need thereof.

In some aspects, the present disclosure provides a peptide of the present disclosure or an isomer, pharmaceutically acceptable salt, or prodrug thereof for use in treating a mental health disorder in a subject in need thereof.

In some aspects, the present disclosure provides a peptide of the present disclosure or an isomer, pharmaceutically acceptable salt, or prodrug thereof for use in treating a metabolic syndrome in a subject in need thereof.

In some aspects, the present disclosure provides a peptide of the present disclosure or an isomer, pharmaceutically acceptable salt, or prodrug thereof for use in treating osteoporosis in a subject in need thereof.

In some aspects, the present disclosure provides a peptide of the present disclosure or an isomer, pharmaceutically acceptable salt, or prodrug thereof for use in treating cardiac failure in a subject in need thereof.

In some aspects, the present disclosure provides a peptide of the present disclosure or an isomer, pharmaceutically acceptable salt, or prodrug thereof for use in treating coma in a subject in need thereof.

In some aspects, the present disclosure provides a peptide of the present disclosure or an isomer, pharmaceutically acceptable salt, or prodrug thereof for use in treating a complication in emergence from anesthesia in a subject in need thereof.

In some aspects, the present disclosure provides use of a peptide of the present disclosure or an isomer, pharmaceutically acceptable salt, or prodrug thereof in the manufacture of a medicament for modulating orexin activity (e.g., in vitro or in vivo).

In some aspects, the present disclosure provides use of a peptide of the present disclosure or an isomer, pharmaceutically acceptable salt, or prodrug thereof in the manufacture of a medicament for modulating orexin-2 activity (e.g., in vitro or in vivo).

In some aspects, the present disclosure provides use of a peptide of the present disclosure or an isomer, pharmaceutically acceptable salt, or prodrug thereof in the manufacture of a medicament for treating or preventing a disease or disorder disclosed herein.

In some aspects, the present disclosure provides use of a peptide of the present disclosure or an isomer, pharmaceutically acceptable salt, or prodrug thereof in the manufacture of a medicament for treating a disease or disorder disclosed herein.

In some aspects, the present disclosure provides use of a peptide of the present disclosure or an isomer, pharmaceutically acceptable salt, or prodrug thereof in the manufacture of a medicament for treating or preventing narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof.

In some aspects, the present disclosure provides use of a peptide of the present disclosure or an isomer, pharmaceutically acceptable salt, or prodrug thereof in the manufacture of a medicament for treating or preventing narcolepsy in a subject in need thereof.

In some aspects, the present disclosure provides use of a peptide of the present disclosure or an isomer, pharmaceutically acceptable salt, or prodrug thereof in the manufacture of a medicament for treating or preventing a hypersomnia disorder in a subject in need thereof.

In some aspects, the present disclosure provides use of a peptide of the present disclosure or an isomer, pharmaceutically acceptable salt, or prodrug thereof in the manufacture of a medicament for treating or preventing a neurodegenerative disorder in a subject in need thereof.

In some aspects, the present disclosure provides use of a peptide of the present disclosure or an isomer, pharmaceutically acceptable salt, or prodrug thereof in the manufacture of a medicament for treating or preventing a symptom of a rare genetic disorder in a subject in need thereof.

In some aspects, the present disclosure provides use of a peptide of the present disclosure or an isomer, pharmaceutically acceptable salt, or prodrug thereof in the manufacture of a medicament for treating or preventing a mental health disorder in a subject in need thereof.

In some aspects, the present disclosure provides use of a peptide of the present disclosure or an isomer, pharmaceutically acceptable salt, or prodrug thereof in the manufacture of a medicament for treating or preventing a metabolic syndrome in a subject in need thereof.

In some aspects, the present disclosure provides use of a peptide of the present disclosure or an isomer, pharmaceutically acceptable salt, or prodrug thereof in the manufacture of a medicament for treating or preventing osteoporosis in a subject in need thereof.

In some aspects, the present disclosure provides use of a peptide of the present disclosure or an isomer, pharmaceutically acceptable salt, or prodrug thereof in the manufacture of a medicament for treating or preventing cardiac failure in a subject in need thereof.

In some aspects, the present disclosure provides use of a peptide of the present disclosure or an isomer, pharmaceutically acceptable salt, or prodrug thereof in the manufacture of a medicament for treating or preventing coma in a subject in need thereof.

In some aspects, the present disclosure provides use of a peptide of the present disclosure or an isomer, pharmaceutically acceptable salt, or prodrug thereof in the manufacture of a medicament for treating or preventing a complication in emergence from anesthesia in a subject in need thereof.

In some aspects, the present disclosure provides use of a peptide of the present disclosure or an isomer, pharmaceutically acceptable salt, or prodrug thereof in the manufacture of a medicament for treating narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof.

In some aspects, the present disclosure provides use of a peptide of the present disclosure or an isomer, pharmaceutically acceptable salt, or prodrug thereof in the manufacture of a medicament for treating narcolepsy in a subject in need thereof.

In some aspects, the present disclosure provides use of a peptide of the present disclosure or an isomer, pharmaceutically acceptable salt, or prodrug thereof in the manufacture of a medicament for treating a hypersomnia disorder in a subject in need thereof.

In some aspects, the present disclosure provides use of a peptide of the present disclosure or an isomer, pharmaceutically acceptable salt, or prodrug thereof in the manufacture of a medicament for treating a neurodegenerative disorder in a subject in need thereof.

In some aspects, the present disclosure provides use of a peptide of the present disclosure or an isomer, pharmaceutically acceptable salt, or prodrug thereof in the manufacture of a medicament for treating a symptom of a rare genetic disorder in a subject in need thereof.

In some aspects, the present disclosure provides use of a peptide of the present disclosure or an isomer, pharmaceutically acceptable salt, or prodrug thereof in the manufacture of a medicament for treating a mental health disorder in a subject in need thereof.

In some aspects, the present disclosure provides use of a peptide of the present disclosure or an isomer, pharmaceutically acceptable salt, or prodrug thereof in the manufacture of a medicament for treating a metabolic syndrome in a subject in need thereof.

In some aspects, the present disclosure provides use of a peptide of the present disclosure or an isomer, pharmaceutically acceptable salt, or prodrug thereof in the manufacture of a medicament for treating osteoporosis in a subject in need thereof.

In some aspects, the present disclosure provides use of a peptide of the present disclosure or an isomer, pharmaceutically acceptable salt, or prodrug thereof in the manufacture of a medicament for treating cardiac failure in a subject in need thereof.

In some aspects, the present disclosure provides use of a peptide of the present disclosure or an isomer, pharmaceutically acceptable salt, or prodrug thereof in the manufacture of a medicament for treating coma in a subject in need thereof.

In some aspects, the present disclosure provides use of a peptide of the present disclosure or an isomer, pharmaceutically acceptable salt, or prodrug thereof in the manufacture of a medicament for treating a complication in emergence from anesthesia in a subject in need thereof.

The present disclosure provides peptides that function as modulators of orexin receptor activity.

In some embodiments, the peptides of the present disclosure are agonists of the orexin receptor.

The present disclosure provides peptides that function as modulators of orexin-2 receptor activity.

In some embodiments, the peptides of the present disclosure are agonists of the orexin-2 receptor.

In some embodiments, the modulation of the orexin receptor is activation of the orexin receptor.

Effectiveness of peptides of the disclosure can be determined by industry-accepted assays/disease models according to standard practices of elucidating the same as described in the art and are found in the current general knowledge.

The present disclosure also provides a method of treating a disease or disorder in which orexin receptor activity is implicated in a patient in need of such treatment, said method comprising administering to said patient a therapeutically effective amount of a peptide, or an isomer, pharmaceutically acceptable salt, or prodrug thereof, or a pharmaceutical composition as defined herein.

The present disclosure also provides a method of treating a disease or disorder in which orexin-2 receptor activity is implicated in a patient in need of such treatment, said method comprising administering to said patient a therapeutically effective amount of a peptide, or an isomer, pharmaceutically acceptable salt, or prodrug thereof, or a pharmaceutical composition as defined herein.

In some embodiments, the present disclosure also provides a method for treating a disease or disorder by decreasing excessive sleepiness and/or excessive daytime sleepiness.

In some embodiments, the present disclosure also provides a method for treating a disease or disorder by decreasing excessive sleepiness.

In some embodiments, the present disclosure also provides a method for treating a disease or disorder by decreasing excessive daytime sleepiness.

In some embodiments, the disease or disorder is associated with excessive sleepiness and/or excessive daytime sleepiness.

In some embodiments, the disease or disorder is a primary hypersomnia disorder, neurodegenerative disorder, a symptom of a hypersomnia/neurodegenerative disorder, a symptom of a rare genetic disorder, a mental health disorder, a metabolic syndrome, osteoporosis, cardiac failure, coma, or emergence from anesthesia.

In some embodiments, the disease or disorder is a primary hypersomnia disorder, neurodegenerative disorder, a symptom of a hypersomnia/neurodegenerative disorder, a symptom of a rare genetic disorder, a mental health disorder, a metabolic syndrome, osteoporosis, cardiac failure, coma, or a complication in emergence from anesthesia.

In some embodiments, the excessive daytime sleepiness is associated with a neurodegenerative disorder.

In some embodiments, the neurodegenerative disorder associated with excessive daytime sleepiness is Parkinson's disease, Alzheimer's disease, Huntington's disease, or multiple sclerosis.

In some embodiments, the disease or disorder is a recurrence of hypersomnia.

In some embodiments, the recurrence of hypersomnia is narcolepsy type 1, narcolepsy type 2, or idiopathic hypersomnia.

In some embodiments, the disease or disorder is sleep apnea, traumatic brain injury, age-related cognitive dysfunction, or excessive daytime sleepiness.

In some embodiments, excessive daytime sleepingess is associated with sleep apnea, traumatic brain injury, or age-related cognitive dysfunction.

In some embodiments, the disorder is narcolepsy. In some embodiments, narcolepsy is narcolepsy type 1. In some embodiments, the narcolepsy is narcolepsy type 2.

In some embodiments, the hypersomnia is a symptom of narcolepsy.

In some embodiments, the disease or disorder is a symptom of narcolepsy.

In some embodiments, a symptom of narcolepsy is excessive daytime sleepiness, cataplexy, sleep paralysis, hypnopompic and hypnogogic hallucinations, disturbed nighttime sleep, or inappropriately timed rapid-eye-movement (REM) sleep.

In some embodiments, a symptom of narcolepsy is excessive daytime sleepiness.

In some embodiments, the symptom of narcolepsy is cataplexy. In some embodiments, cataplexy is pathognomonic of narcolepsy (e.g., narcolepsy type 1)

In some embodiments, a symptom of narcolepsy is sleep paralysis.

In some embodiments, a symptom of narcolepsy is hypnopompic and hypnogogic hallucinations.

In some embodiments, a symptom of narcolepsy is disturbed nighttime sleep.

In some embodiments, a symptom of narcolepsy is inappropriately timed rapid-eye-movement (REM) sleep.

In some embodiments, the neurodegenerative disorder is characterized by cataplexy.

In some embodiments, the neurodegenerative disorder is characterized by excessive daytime sleepiness.

In some embodiments, the neurodegenerative disorder is Parkinson's disease.

In some embodiments, the neurodegenerative disorder is Alzheimer's disease.

In some embodiments, the neurodegenerative disorder is Huntington's disease.

In some embodiments, the neurodegenerative disorder is multiple sclerosis.

In some embodiments, the neurodegenerative disorder is a traumatic brain injury.

In some embodiments, the neurodegenerative disorder is sleep apnea.

In some embodiments, the neurodegenerative disorder is age-related cognitive dysfunction.

In some embodiments, the neurodegenerative disorder is a disorder of recurrent hypersomnia.

In some embodiments, a disorder of recurrent hypersomnia is Klein-Levin syndrome, inappropriately timed sleep, (e.g., delayed- or advanced-sleep phase disorder), shift work disorder, or jet lag disorder.

In some embodiments, the disease or disorder is a symptom of a rare genetic disorder.

In some embodiments, a symptom of a rare genetic disorder is abnormal daytime sleepiness.

In some embodiments, a symptom of a rare genetic disorder is excessive daytime sleepiness.

In some embodiments, a symptom of a rare genetic disorder is sleep onset REM periods.

In some embodiments, a symptom of a rare genetic disorder is characterized by cataplexy-like symptoms.

In some embodiments, a rare genetic disorder is Myotonic Dystrophy, ADCA-DN, Coffin-Lowry syndrome, Moebius syndrome, Norrie disease, Niemann-Pick disease type C, or Prader-Willi syndrome.

In some embodiments, the disease or disorder is a mental health disorder.

In some embodiments, the mental health disorder is attention deficit hyperactivity disorder.

In some embodiments, the mental health disorder is attention deficit disorder.

In some embodiments, the disease or disorder is a metabolic syndrome.

In some embodiments, the metabolic syndrome is obesity.

In some embodiments, the disease or disorder is osteoporosis.

In some embodiments, the disease or disorder is cardiac failure.

In some embodiments, the disease or disorder is a coma.

In some embodiments, the disease or disorder is emergence from anesthesia.

In some embodiments, the disease or disorder is a complication in emergence from anesthesia.

In some embodiments, the disease or disorder is narcolepsy, a hypersomnia disorder, a neurodegenerative disorder, a neurological disorder, a symptom of a rare genetic disorder, a psychiatric disorder, a mental health disorder, a circadian rhythm disorder, a metabolic syndrome, osteoporosis, cardiac failure, coma, or a complication in emergence from anesthesia.

In some embodiments, the disease or disorder is narcolepsy, idiopathic hypersomnia, or sleep apnea.

### Routes of Administration

Peptides of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the peptide of Construct Number 1, Construct Number 2, or Construct Number 3 with another therapeutic agent (which also includes a therapeutic regimen) that also has therapeutic benefit.

In the instances where the peptide of the present disclosure is administered in combination with other therapeutic agents, the peptide of the disclosure need not be administered via the same route as other therapeutic agents, and may, because of different physical and chemical characteristics, be administered by a different route. For example, the peptide of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which orexin activity is implicated comprising a peptide of the disclosure as defined hereinbefore, or an isomer, pharmaceutically acceptable salt, or prodrug thereof, and another suitable agent.

According to a further aspect of the disclosure there is provided a pharmaceutical composition which comprises a peptide of the disclosure, or an isomer, pharmaceutically acceptable salt, or prodrug thereof, in combination with a suitable, in association with a pharmaceutically acceptable diluent or carrier.

In addition to its use in therapeutic medicine, peptides of Construct Number 1, Construct Number 1, or Construct Number 3 and pharmaceutically acceptable salts thereof are also useful as pharmacological tools in the development and standardisation of in vitro and in vivo test systems for the evaluation of the effects of modulators of orexin receptor activity in laboratory animals such as dogs, rabbits, monkeys, rats and mice, as part of the search for new therapeutic agents.

In addition to its use in therapeutic medicine, peptides of Construct Number 1, Construct Number 2, or Construct Number 3 and pharmaceutically acceptable salts thereof are also useful as pharmacological tools in the development and standardisation of in vitro and in vivo test systems for the evaluation of the effects of modulators of orexin-2 receptor activity in laboratory animals such as dogs, rabbits, monkeys, rats and mice, as part of the search for new therapeutic agents.

In some embodiments, the peptides with a 14 amino acid sequence have sufficient potency.

In some embodiments, the peptides with a 15 amino acid sequence have sufficient potency.

In some embodiments, the peptides with a 13 amino acid sequence do not have sufficient potency.

In any of the above-mentioned pharmaceutical composition, process, method, use, medicament, and manufacturing features of the instant disclosure, any of the alternate embodiments of molecules of the present disclosure described herein also apply.

The peptides of the disclosure or pharmaceutical compositions comprising these peptides may be administered to a subject by any convenient route of administration, whether systemically, peripherally, or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray or powder); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Exemplary Embodiments

Exemplary Embodiment 1. A peptide having a sequence comprising:

Z₁X₁X₂X₃X₄X₅X₆X₇X₈X₉X₁₀X₁₁X₁₂X₁₃X₁₄X₁₅-NH₂
(Construct Number 1),

or an isomer, pharmaceutically acceptable salt, or prodrug thereof, wherein,
Z₁ is absent, a capping group, Arg, or hArg(Et)₂;
X₁ is absent, an amino acid comprising a hydrophobic side chain, or a derivative thereof, or a polar uncharged side chain, or a derivative thereof;
X₂ is an amino acid comprising a polar uncharged side chain, or a derivative thereof, a hydrophobic side chain, or a derivative thereof, or an electrically charged side chain, or a derivative thereof;
X₃ is an amino acid comprising an electrically charged side chain, or a derivative thereof, a polar uncharged side chain, or a derivative thereof, or a hydrophobic side chain, or a derivative thereof;
X₄ is an amino acid comprising a polar charged side chain, or a derivative thereof, or a hydrophobic side chain, or a derivative thereof;
X₅ is an amino acid comprising an electrically charged side chain, or a derivative thereof, or a polar uncharged side chain, or a derivative thereof;
X₆ is Gly or a Gly derivative;
X₇ is an amino acid comprising a polar uncharged side chain, or a derivative thereof;
X₈ is an amino acid comprising a polar charged side chain, or a derivative thereof, or a hydrophobic side chain, or a derivative thereof;
X₉ is an amino acid comprising a hydrophobic side chain, or a derivative thereof;
X₁₀ is Ala, Gly, or a Gly derivative;
X₁₁ is Gly or a Gly derivative;
X₁₂ is an amino acid comprising a hydrophobic side chain, or a derivative thereof;
X₁₃ is an amino acid comprising a hydrophobic side chain, or a derivative thereof;
X₁₄ is an amino acid comprising a polar uncharged side chain, or a derivative thereof; and
X₁₅ is an amino acid comprising a hydrophobic side chain or a derivative thereof.
Exemplary Embodiment 2. A peptide having a sequence comprising:

Z₁X₁X₂X₃X₄X₅X₆X₇X₈X₉X₁₀X₁₁X₁₂X₁₃X₁₄X₁₅-NH₂
(Construct Number 2),

or an isomer, pharmaceutically acceptable salt, or prodrug thereof, wherein,
Z₁ is absent, a capping group, Arg, or hArg(Et)₂;
X₁ is absent, an amino acid comprising a hydrophobic side chain, or a derivative thereof, or a polar uncharged side chain, or a derivative thereof;
X₂ is an amino acid comprising a polar uncharged side chain, or a derivative thereof, a hydrophobic side chain, or a derivative thereof, or an electrically charged side chain, or a derivative thereof;
X₃ is an amino acid comprising an electrically charged side chain, or a derivative thereof, a polar uncharged side chain, or a derivative thereof, or a hydrophobic side chain, or a derivative thereof;
X₄ is an amino acid comprising a polar charged side chain, or a derivative thereof, or a hydrophobic side chain, or a derivative thereof;
X₅ is an amino acid comprising an electrically charged side chain, or a derivative thereof, or a polar uncharged side chain, or a derivative thereof;
X₆ is Gly or a Gly derivative;
X₇ is an amino acid comprising a polar uncharged side chain, or a derivative thereof;
X₈ is an amino acid comprising a polar charged side chain, or a derivative thereof, or a hydrophobic side chain, or a derivative thereof;
X₉ is an amino acid comprising a hydrophobic side chain, or a derivative thereof;
X₁₀ is Ala, Gly, or a Gly derivative;
X₁₁ is Gly or a Gly derivative;
X₁₂ is an amino acid comprising a hydrophobic side chain, or a derivative thereof;
X₁₃ is an amino acid comprising a hydrophobic side chain, or a derivative thereof;
X₁₄ is an amino acid comprising a polar uncharged side chain, or a derivative thereof; and
X₁₅ is 2-AOC, 2-AHP, NLE, NVA, Phe or Phe(3-Br), wherein the phenyl of X₁₅ is optionally substituted with halo, —OH, —O(C₁-C₆alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.
Exemplary Embodiment 3. A peptide having a sequence comprising:

Z₁X₁X₂X₃X₄X₅X₆X₇X₈X₉X₁₀X₁₁X₁₂X₁₃X₁₄X₁₅-NH₂
(Construct Number 3),

Z₁ is absent, a capping group, Arg, or hArg(Et)₂;
X₁ is absent, Phe, Leu, Gln, D-Gln, or Hyp(4-OH);
X₂ is Gln, Asn, N-Me-Asn, Thr, Ser, D-Gln, D-Leu, Leu, hArg, D-hArg, or Hyp(4-OH);
X₃ is Arg, hArg(Et)₂, Hyp(4-OH), His, Lys, Asp, Glu, Ser, D-Phe, Trp, hArg, Pro, D-Pro, or D-hArg;
X₄ is Hyp(4-OH), His, Arg, Lys, Asp, Gln, Glu, Pro, D-Pro, Ala, or D-Ala;
X₅ is Arg, hArg(Et)₂, Hyp(4-OH), His, Lys, Asp, Glu, or Ser;
X₆ is N-Phenethyl-Gly, N-(naphthalen-2-yl-ethyl)-Gly, N-(naphthalen-1-yl-ethyl)-Gly, N-(3-EtNH₂-Phenethyl)-Gly, N-(4-OMe-Phenethyl)-Gly, Phg, Phg(4-OH), or Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₇ is Asn, N-Me-Asn, Ser, Thr, or Gln;
X₈ is Hyp(4-OH), His, Arg, Lys, Asp, Gln, or Glu;
X₉ is Ala, Val, Ile, Leu, Met, Phe, Tyr, or Trp, wherein the phenyl of X₉ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₀ is Phg, Phg(4-OH), N-Phenethyl-Gly, N(naphtha-2-yl-ethyl)-Gly, N-(4-OMe-Phenethyl)-Gly, Gly, or Ala, wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₁ is Phg, Phg(4-OH), N-Phenethyl-Gly, N(naphtha-2-yl-ethyl)-Gly, N-(4-OMe-Phenethyl)-Gly, or Gly, wherein the phenyl of X₁₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₂ is Ile, Ala, Val, Leu, Met, Phe, Tyr, or Trp, wherein the phenyl of X₁₂ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₃ is N-Me-Leu, Leu, Ala, Val, Ile, Met, Phe, Tyr, or Trp, wherein the phenyl of X₁₃ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₄ is Thr, Ser, Asn, or Gln; and
X₁₅ is 2-AOC, 2-AHP, NLE, NVA, Phe, or Phe(3-Br), wherein the phenyl of X₁₅ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.
Exemplary Embodiment 4. The peptide of any one of the preceding Exemplary Embodiments, wherein:
   Z₁ is absent or CAP;
   X₁ is absent, Phe, Leu, Gln, D-Gln, or Hyp(4-OH);
   X₂ is Gln, D-Gln, Hyp(4-OH), D-Leu, hArg, D-hArg, or Leu;
   X₃ is Hyp(4-OH), D-Phe, Trp, hArg, Pro, D-Pro, or D-hArg;
   X₄ is Hyp(4-OH), Arg, Pro, D-Pro, Ala, or D-Ala;
   X₅ is Arg, hArg(Et)₂, Lys, or Ser;
   X₆ is N-Phenethyl-Gly or Gly;
   X₇ is Asn or N-Me-Asn;
   X₈ is Hyp(4-OH) or His;
   X₉ is Ala;
   X₁₀ is Phg or Ala;
   X₁₁ is Gly;
   X₁₂ is Ile;
   X₁₃ is Leu;
   X₁₄ is Thr; and
   X₁₅ is 2-AOC or NLE.
Exemplary Embodiment 5. The peptide of any one of the preceding Exemplary Embodiments, wherein Z₁ is CAP.
Exemplary Embodiment 6. The peptide of any one of the preceding Exemplary Embodiments, wherein Z₁ is Arg or hArg(Et)₂.
Exemplary Embodiment 7. The peptide of any one of the preceding Exemplary Embodiments, wherein X₁ is absent.
Exemplary Embodiment 8. The peptide of any one of the preceding Exemplary Embodiments, wherein X₁ is Phe, Leu, or Hyp(4-OH).
Exemplary Embodiment 9. The peptide of any one of the preceding Exemplary Embodiments, wherein X₂ is Gln, Asn, N-Me-Asn, Thr, Ser, D-Gln, D-Leu, Leu, or Hyp(4-OH).
Exemplary Embodiment 10. The peptide of any one of the preceding Exemplary Embodiments, wherein X₂ is Gln, D-Gln, Hyp(4-OH), D-Leu, or Leu.
Exemplary Embodiment 11. The peptide of any one of the preceding Exemplary Embodiments, wherein X₃ is Hyp(4-OH), D-Phe, Trp, hArg, or D-hArg.
Exemplary Embodiment 12. The peptide of any one of the preceding Exemplary Embodiments, wherein X₄ is Hyp(4-OH), His, Arg, Lys, Asp, Gln, Glu, Pro, D-Pro, Ala, or D-Ala.
Exemplary Embodiment 13. The peptide of any one of the preceding Exemplary Embodiments, wherein X₅ is Arg, hArg(Et)₂, Hyp(4-OH), His, Lys, Asp, Glu, or Ser.
Exemplary Embodiment 14. The peptide of any one of the preceding Exemplary Embodiments, wherein X₅ is Arg, hArg(Et)₂, Lys, or Ser.
Exemplary Embodiment 15. The peptide of any one of the preceding Exemplary Embodiments, wherein X₆ is N-Phenethyl-Gly, N-(naphthalen-2-yl-ethyl)-Gly, N-(naphthalen-1-yl-ethyl)-Gly, N-(3-EtNH₂-Phenethyl)-Gly, N-(4-OMe-Phenethyl)-Gly, Phg, Phg(4-OH), or Gly.
Exemplary Embodiment 16. The peptide of any one of the preceding Exemplary Embodiments, wherein X₆ is N-Phenethyl-Gly or Gly.
Exemplary Embodiment 17. The peptide of any one of the preceding Exemplary Embodiments, wherein X₇ is Asn, N-Me-Asn, Ser, Thr, or Gln.
Exemplary Embodiment 18. The peptide of any one of the preceding Exemplary Embodiments, wherein X₇ is Asn or N-Me-Asn.
Exemplary Embodiment 19. The peptide of any one of the preceding Exemplary Embodiments, wherein X₈ is Hyp(4-OH), His, Arg, Lys, Asp, Gln, or Glu.
Exemplary Embodiment 20. The peptide of any one of the preceding Exemplary Embodiments, wherein X₈ is Hyp(4-OH) or His.
Exemplary Embodiment 21. The peptide of any one of the preceding Exemplary Embodiments, wherein X₉ is Ala.
Exemplary Embodiment 22. The peptide of any one of the preceding Exemplary Embodiments, wherein X₁₀ is Phg.
Exemplary Embodiment 23. The peptide of any one of the preceding Exemplary Embodiments, wherein X₁₁ is Gly.
Exemplary Embodiment 24. The peptide of any one of the preceding Exemplary Embodiments, wherein X₁₂ is Ile.
Exemplary Embodiment 25. The peptide of any one of the preceding Exemplary Embodiments, wherein Xu is Leu.
Exemplary Embodiment 26. The peptide of any one of the preceding Exemplary Embodiments, wherein X₁₄ is Thr.
Exemplary Embodiment 27. The peptide of any one of the preceding Exemplary Embodiments, wherein X₁₅ is 2-AOC, 2-AHP, NLE, NVA, Phe or Phe(3-Br).
Exemplary Embodiment 28. The peptide of any one of the preceding Exemplary Embodiments, wherein X₁₅ is 2-AOC or NLE.
Exemplary Embodiment 29. The peptide of any one of the preceding Exemplary Embodiments, wherein the capping group is

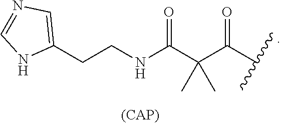 [SMILES: *C(=O)C(C)(C)C(=O)NCCc1cnc[nH]1]

Exemplary Embodiment 30. The peptide of any one of the preceding Exemplary Embodiments, being of Construct Number 4 or SEQ ID NO: 5:

H₂N-(PEG)₆-X₁X₂X₃X₄X₅X₆X₇X₈X₉X₁₀X₁₁X₁₃X₁₄-
(2-AOC)-NH₂
(Construct Number 4),
(SEQ ID NO: 5)
Z₁X₁X₂X₃X₄X₅X₆X₇-Hyp(4-OH)-ALA-Phg-GLY-ILE-
LEU-THR-X₁₅-NH₂,

or an isomer, pharmaceutically acceptable salt, or prodrug thereof.
Exemplary Embodiment 31. The peptide of any one of the preceding Exemplary Embodiments, being of SEQ ID NO: 6:

(SEQ ID NO: 6)
H₂N-(PEG)₆-X₁X₂X₃X₄X₅X₆X₇-Hyp(4-OH)-ALA-
Phg-GLY-ILE-LEU-THR-(2-AOC)-NH₂,

or an isomer, pharmaceutically acceptable salt, or prodrug thereof.
Exemplary Embodiment 32. The peptide of any one of the preceding Exemplary Embodiments, wherein the peptide is selected from the peptides described in Table 1 and pharmaceutically acceptable salts thereof.
Exemplary Embodiment 33. A peptide obtainable by, or obtained by, a method described herein.
Exemplary Embodiment 34. A pharmaceutical composition comprising the peptide of any one of the preceding Exemplary Embodiments or an isomer, pharmaceutically acceptable salt, or prodrug thereof, and a pharmaceutically acceptable diluent or carrier.
Exemplary Embodiment 35. The pharmaceutical composition of any one of the preceding Exemplary Embodiments, wherein the peptide is selected from the peptides described in Table 1.
Exemplary Embodiment 36. A method of modulating orexin receptor activity, comprising contacting a cell with an effective amount of the peptide of any one of the preceding Exemplary Embodiments; optionally the activity is in vitro or in vivo.
Exemplary Embodiment 37. A method of treating or preventing a disease or disorder in a subject in need thereof, comprising administering to the subject a therapeutically effective amount of the peptide or pharmaceutical composition of any one of the preceding Exemplary Embodiments.
Exemplary Embodiment 38. The peptide or pharmaceutical composition of any one of the preceding Exemplary Embodiments for use in modulating orexin receptor activity.
Exemplary Embodiment 39. The peptide or pharmaceutical composition of any one of the preceding Exemplary Embodiments for use in treating or preventing a disease or disorder.
Exemplary Embodiment 40. Use of the peptide of any one of the preceding Exemplary Embodiments in the manufacture of a medicament for modulating orexin receptor activity.
Exemplary Embodiment 41. Use of the peptide of any one of the preceding Exemplary Embodiments in the manufacture of a medicament for treating or preventing a disease or disorder.
Exemplary Embodiment 42. The method, peptide, pharmaceutical composition, or use of any one of the preceding Exemplary Embodiments, wherein the disease or disorder is associated with an implicated orexin receptor.
Exemplary Embodiment 43. The method, peptide, pharmaceutical composition, or use of any one of the preceding Exemplary Embodiments, wherein the disease or disorder is associated with an implicated orexin-2 receptor.
Exemplary Embodiment 44. The method, peptide, pharmaceutical composition, or use of any one of the preceding Exemplary Embodiments, wherein the disease or disorder is narcolepsy, a hypersomnia disorder, a neurodegenerative disorder, a neurological disorder, a symptom of a rare genetic disorder, a psychiatric disorder, a mental health disorder, a circadian rhythm disorder, a metabolic syndrome, osteoporosis, cardiac failure, coma, or a complication in emergence from anesthesia.
Exemplary Embodiment 45. The method, peptide, pharmaceutical composition, or use of any one of the preceding Exemplary Embodiments, wherein the disease or disorder is narcolepsy, idiopathic hypersomnia, or sleep apnea.

### EXAMPLES

### General Procedures

Where no preparative routes are included, the relevant intermediate is commercially available. Commercial reagents were utilized without further purification. Room temperature (rt) refers to approximately 20-27° C. ¹H NMR spectra were recorded at 400 MHz on a Bruker instrument. Chemical shift values are expressed in parts per million (ppm), i.e. (6)-values. The following abbreviations are used for the multiplicity of the NMR signals: s=singlet, br=broad, d=doublet, t=triplet, q=quartet, quint=quintet, td=triplet of doublets, tt=triplet of triplets, qd=quartet of doublets, ddd=doublet of doublet of doublets, ddt=doublet of doublet of triplets, m=multiplet. Coupling constants are listed as J values, measured in Hz. NMR and mass spectroscopy results were corrected to account for background peaks.

### Abbreviations

aq aqueous
DCM dichloromethane
DMF N,N-dimethylformamide
DIC N,N′-Diisopropylcarbodiimide
DIEA N,N-diisopropylethylamine
DMSO dimethylsulfoxide
DTT dithiothreitol
EtOAc ethyl acetate
Fmoc fluorenylmethoxycarbonyl
h hour(s)
HATU 1-[bis(dimethylamino)methylene]-1H-1,2,3-triazolo[4,5-b]pyridinium 3-oxide hexafluorophosphate
HBTU (2-(1H-benzotriazol-1-yl)-1,1,3,3-tetramethyluronium hexafluorophosphate
HOBt hydroxybenzotriazole
HPLC high performance liquid chromatography
LC liquid chromatography
MeCN acetonitrile
MS mass spectrometry
NMR nuclear magnetic resonance
Pbf 2,2,4,6,7-pentamethyldihydrobenzofuran-5-sulfonyl
PDA photo diode array
rt room temperature
s second(s)
SQD single quad detector
SPPS solid phase peptide synthesis
TFA trifluoroacetic acid
THF tetrahydrofuran
Tis triisopylsilane
Trt or trityl triphenylmethyl
UPLC ultra performance liquid chromatography
   Prefixes n-, s-, i-, t- and tert- have their usual meanings: normal, secondary, iso, and tertiary.

### Analytical Methods

LCMS Method A: Instrument: Waters Acquity UPLC, Waters 3100 PDA Detector, SQD; Column: Acquity HSS-T3, 1.8 micron, 2.1×100 mm; Gradient [time (min)/solvent B in A (%)]: 0.00/10, 1.00/10, 2.00/15, 4.50/55, 6.00/90, 8.00/90, 9.00/10, 10.00/10; Solvents: solvent A=0.1% TFA acid in water; solvent B=MeCN; Injection volume 1 L; Detection wavelength 214 nm; Column temperature 30° C.; Flow rate 0.3 mL per min.

LCMS Method B: Instruments: Waters Acquity H-Class LCMS, PDA Detector and QDa Mass Detector, Mass Lynx software; Column: Gemini-NX C18, 3 μm, 30×2 mm; Gradient [time (min)/solvent B in A (%)]: 0.00/0, 1.30/100, 1.55/100, 1.60/0, 3.00/0; Solvents: solvent A=50 mM ammonium acetate aqueous solution at pH 7.4; solvent B=MeCN; Injection volume 1 μL; Detection wavelength range 200-500 nm; Column temperature 40° C.; Flow rate 0.5 mL per min.

### Synthesis of Intermediates and Peptides

The following examples are provided to illustrate preferred aspects of the invention and are not intended to limit the scope of the invention.

### Synthesis of Intermediates

All Fmoc-amino acids are commercially available except for intermediate 1.

### Example 1. Synthesis of 2,2-dimethyl-3-oxo-3-((2-(1-trityl-1H-imidazol-4-yl)ethyl)amino)propanoic acid (Intermediate 1)

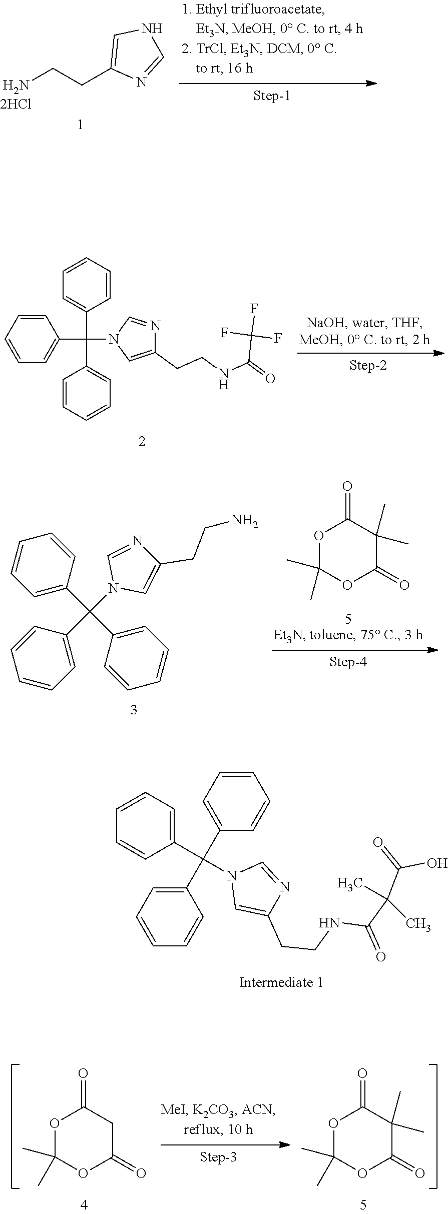

Step-1: Synthesis of 2,2,2-trifluoro-N-(2-(1-trityl-1H-imidazol-4-yl)ethyl)acetamide (2): To a solution of 2-(1H-imidazol-4-yl)ethan-1-amine dihydrochloride (1, 25.0 g, 136.6 mmol) in MeOH (100 mL), Et₃N (67 mL, 464.4 mmol) was added at rt and the reaction mixture was cooled to 0° C. A solution of ethyl trifluoroacetate (20 mL, 164.0 mmol) in MeOH (50 mL) was added to the reaction mixture over 30 min at 0° C. and the reaction mixture was stirred at rt for 4 h. This reaction mixture was diluted with dry DCM (200 mL) and Et₃N (60 mL, 409.8 mmol) and the reaction mixture was cooled to 0° C. Trityl chloride (76 g, 273.2 mmol) was added portion wise and the resulting reaction mixture was stirred at rt for 16 h. After completion, the reaction mixture was quenched with water (300 mL) and the aqueous layer was extracted with chloroform (3×150 mL). The organic layers were combined, dried (Na₂SO₄) and concentrated in vacuo. The crude residue was triturated with n-hexane to give 2,2,2-trifluoro-N-(2-(1-trityl-1H-imidazol-4-yl)ethyl)acetamide (2, 50.10 g, 81%) as a white solid.

MS (ESI+ve): 450

¹H-NMR (400 MHz; CDCl₃): δ 2.75 (t, J=5.9 Hz, 2H), 3.60-3.65 (m, 2H), 6.61 (s, 1H), 7.08-7.15 (m, 6H), 7.31-7.38 (m, 9H), 7.40 (s, 1H), 8.41 (bs, 1H).

Step-2: Synthesis of 2-(1-trityl-1H-imidazol-4-yl)ethan-1-amine (3): To a solution of 2,2,2-trifluoro-N-(2-(1-trityl-1H-imidazol-4-yl)ethyl)acetamide (2, 50.0 g, 111.3 mmol) in THF (150 mL) and MeOH (180 mL), NaOH (22.0 g, 556.7 mmol) in water (100 mL) was slowly added at 0° C. and the reaction mixture was stirred at rt for 2 h. After completion, the reaction mixture was quenched with water (300 mL) and the aq. layer was extracted with chloroform (3×150 mL). The organic layers were combined, dried (Na₂SO₄) and concentrated in vacuo to give 2-(1-trityl-1H-imidazol-4-yl)ethan-1-amine (3, 34.0 g, 86%) as a yellowish sticky solid. The crude residue was used for the next step without further purification.

MS (ESI+ve): 354

¹H-NMR (400 MHz; CDCl₃): δ 1.53 (bs, 2H), 2.65 (t, J=6.5 Hz, 2H), 2.95 (t, J=6.5 Hz, 2H), 6.58 (s, 1H), 7.11-7.16 (m, 6H), 7.28-7.38 (m, 10H).

Step-3: Synthesis of 2,2,5,5-tetramethyl-1,3-dioxane-4,6-dione (5): To a solution of 2,2-dimethyl-1,3-dioxane-4,6-dione (4, 20.0 g, 138.8 mmol) in MeCN (200 mL), K₂CO₃ (96 g, 694.0 mmol) and Mel (26 mL, 416.6 mmol) were added at rt and reaction mixture was refluxed for 10 h. After completion, the reaction mixture was cooled to rt, filtered through a pad of celite, washed with EtOAc (3×50 mL). The organic layer was washed with 10% aq. Na₂S₂O₃ (100 mL), dried, (Na₂SO₄) and concentrated in vacuo to give 2,2,5,5-tetramethyl-1,3-dioxane-4,6-dione (5, 21 g, 88%) as a yellow solid. The crude residue was used for the next step without further purification.

¹H-NMR (400 MHz; CDCl₃): δ 1.63 (s, 6H), 1.73 (s, 6H).

Step-4: Synthesis of 2,2-dimethyl-3-oxo-3-((2-(1-trityl-1H-imidazol-4-yl)ethyl)amino) propanoic acid (Intermediate 1): A solution of 2-(1-trityl-1H-imidazol-4-yl)ethan-1-amineto (3, 8.0 g, 22.6 mmol) and Et₃N (16.0 mL, 113.0 mmol) in toluene (100 mL) was added drop wise over 60 min to a solution of 2,2,5,5-tetramethyl-1,3-dioxane-4,6-dione (5, 5.8 g, 29.76 mmol) in toluene (50 mL) at 75° C. The reaction mixture was further stirred at same temperature for 3 h. After completion, the reaction mixture was concentrated in vacuo. The residue was dissolved in chloroform (100 mL) and washed with 10% aq. citric acid (approximate pH 6-6.5). The organic layer was dried (Na₂SO₄) and concentrated in vacuo. The crude residue obtained was triturated with hot chloroform (150 mL) and n-hexane (75 mL) and the suspension was stirred at rt for 16 h. The solid was filtered, washed with chloroform:n-hexane (1:1, 2×50 mL) and dried in vacuo to give 2,2-dimethyl-3-oxo-3-((2-(1-trityl-1H-imidazol-4-yl)ethyl)amino)propanoic acid (Intermediate 1, 6.8 g, 64%) as a white solid.

LCMS (Method A): m/z 468 [M+H]⁺ (ES+), at 5.38 min, 99.31%

1H-NMR (400 MHz; DMSO-d₆): δ 1.21 (s, 6H), 2.57 (t, J=6.8 Hz, 2H), 3.22-3.27 (m, 2H), 6.66 (s, 1H), 7.06-7.11 (m, 6H), 7.28 (s, 1H), 7.35-7.42 (m, 8H), 7.64 (t, J=5.4 Hz, 1H), 8.31 (s, 1H), 12.44 (bs, 1H).

### Example 2. Synthesis of Examples 1-20

The peptide examples described herein may be prepared by chemical synthesis using solid-phase techniques such as those described in G. Barany and R. B. Merrifield, “The Peptides: Analysis, Synthesis, Biology”; Volume 2—“Special Methods in Peptide Synthesis, Part A”, pp. 3-284, E. Gross and J. Meienhofer, Eds., Academic Press, New York, 1980; and in J. M. Stewart and J. D. Young, “Solid-Phase Peptide Synthesis”, 2nd Ed., Pierce Chemical Co., Rockford, 111., 1984. The synthetic strategy is based on the Fmoc (9-Fluorenylmethyloxycarbonyl) group for temporary protection of the α-amino group, in combination with protecting groups such as tert-butyl (-tBu), tert-butyloxycarbonyl (-Boc), trityl (-Trt) groups for temporary protection of the amino acid side chains (see for example E. Atherton and R. C. Sheppard, “The Fluorenylmethoxycarbonyl Amino Protecting Group”, in “The Peptide Analysis, Synthesis, Biology”; Volume 9-“Special Methods in Peptide Synthesis, Part C”, pp. 1-38, S. Undenfriend and J. Meienhofer, Eds., Academic Press, San Diego, 1987). The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C terminal amide or carboxylic acid, respectively. The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

### General Method for Peptide Synthesis:

The general method used for peptide synthesis is detailed below for Example 3.
1) Add DMF to the vessel containing Rink amide MBHA Resin (0.3 mmol, 1.01 g, sub: 0.297 mmol/g) and swell for 2 h.
2) Drain and then wash with DMF for 30 s, 3 times.
3) Add 200 piperidine/DMF and mix for 30 mq.
4) Drain and then wash with DMF for 30 s, 5 times.
5) Add Fmoc-amino acid solution and mix 30 seconds, then add coupling regents, N₂ bubbling for approximately 1 h.
6) Repeat step 2 to 5 for next amino acid coupling.
   The synthesis scale was 0.30 mmol.

| Amino Acid No. | Materials | Coupling reagents |
|---|---|---|
| 1 | Fmoc-AOC-OH (2.00 eq) | HBTU (1.90 eq) and DIEA (4.00 eq) |
| 2 | Fmoc-Thr(tBu)-OH (3.00 eq) | HBTU (2.85 eq) and DIEA (6.00 eq) |
| 3 | Fmoc-Leu-OH (3.00 eq) | HBTU (2.85 eq) and DIEA (6.00 eq) |
| 4 | Fmoc-Ile-OH (3.00 eq) | HBTU (2.85 eq) and DIEA (6.00 eq) |
| 5 | Fmoc-Gly-OH (3.00 eq) | HBTU (2.85 eq) and DIEA (6.00 eq) |
| 6 | Fmoc-Phg-OH (2.00 eq) | HOBt (2.00 eq) and DIC (2.00 eq) |
| 7 | Fmoc-Ala-OH (3.00 eq) | HBTU (2.85 eq) and DIEA (6.00 eq) |
| 8 | Fmoc-HYP(4-OH)(tBu)-OH (3.00 | HATU (2.85 eq) and DIEA (6.00 eq) |
| | eq) | |
| 9 | Fmoc-N-Me-Asn(Trt)-OH (3.00 eq) | HATU (2.85 eq) and DIEA (6.0 eq) |
| 10 | Fmoc-N-Phenethyl-Gly-OH (3.00 | HATU (2.85 eq) and DIEA (6.0 eq) |
| | eq) | |
| 11 | Fmoc-Arg(Pbf)-OH (3.00 eq) | HATU (2.85 eq) and DIEA (6.00 eq) |
| 12 | Fmoc-Pro-OH (3.0 eq) | HATU (2.85 eq) and DIEA (6.0 eq) |
| 13 | Fmoc-hArg(Pbf)-OH (3.0 eq) | HATU (2.85 eq) and DIEA (6.0 eq) |
| 14 | Fmoc-Gln(Trt)-OH (3.0 eq) | HATU (2.85 eq) and DIEA (6.0 eq) |
| 15 | Intermediate 1 (3.0 eq) | HOBt (3.0 eq) and DIC (3.0 eq) |

### Peptide Cleavage and Purification:

1) Add cleavage buffer (92.5% TFA/2.5% TIS/2.5% H₂O/2.5% DTT) to the flask containing the side chain protected peptide at rt and stir for 3 h.
2) Filter and collect the filtrate.
3) The peptide is precipitated with cold isopropyl ether and centrifuged (3 min at 3000 rpm).
4) Wash with isopropyl ether two additional times.
5) Dry the crude peptide in vacuo for 2 h.
6) The crude peptide was purified by Prep-HPLC (Solvents: A: 0.075% TFA in H₂O, B: MeCN) and convert to AcOH salt to give Example 1 (14.1 mg).

### Purification Conditions and Conversion to AcOH Salt:

Purification conditions

| | |
|---|---|
| Dissolution | Dissolve in MeCN/H₂O |
| condition | |
| Instrument | GX-281 |
| Mobile Phase | A: H₂O (0.075% TFA in H₂O) |
| | B: MeCN |
| Gradient | 25-45% Mobile Phase B in A, over 51 min |
| Column | Luna C18, 100*25 mm, 10 μm, 100 Å + Gemini C18, |
| | 150*30 mm, 5 μm, 110 Å |
| Flow Rate | 20 mL/min |
| Wavelength | 220/254 nm |
| Oven | Room temperature |
| Temperature | |

Separation conditions

| | |
|---|---|
| Dissolution | Dissolve in 30% MeCN/H₂O |
| condition | |
| Instrument | GX-281 |
| Mobile Phase | A: H₂O (0.5% AcOH in H₂O) |
| | B: MeCN |
| Gradient | 0.4M aq. NH₄OAc for 30 min, then 100% Solvent A for |
| | 10 min, then 10-70% Mobile Phase B in A over 51 min. |
| Column | Luna 200*25 mm, C18, 10 μm, 100 Å + Gemini 150*30 |
| | mm, C18, 5 μm, 110 Å |
| Flow Rate | 20 mL/min |
| Wavelength | 220/254 nm |
| Oven | Room temperature |
| Temperature | |

Peptide Nos. 1, 2 and 4 to 20 were synthesized and purified using similar procedures to those detailed above for Example 3. For the Examples below, different materials and, or, different coupling agents, and, or, different equivalents of coupling agents were used.

| Peptide No. | Materials | Coupling Reagents |
|---|---|---|
| 10, 13 | Fmoc-Phe-OH (3.0 eq) | HATU (2.85 eq) and DIEA (6.0 eq) |
| 11, 12, 13, | Fmoc-Leu-OH (3.0 eq) | HATU (2.85 eq) and DIEA(6.0 eq) |
| 14 | | |
| 4, 7, 18, 19, | Fmoc-D-Gln(Trt)-OH (3.0 eq) | HATU (2.85 eq) and DIEA (6.0 eq) |
| 20 | | |
| 10, 11, 14 | Fmoc-D-Leu-OH (3.0 eq) | HATU (2.85 eq) and DIEA(6.0 eq) |
| 5, 7 | Fmoc-D-hArg(Pbf)-OH (2.0 eq) | HATU (2.85 eq) and DIEA (6.0 eq) |
| 18 | Fmoc-D-hArg(Pbf)-OH (3.0 eq) | HATU (2.85 eq) and DIEA (6.0 eq) |
| 10, 11 | Fmoc-Trp(Boc)-OH (3.0 eq) | HATU (2.85 eq) and DIEA (6.0 eq) |
| 12, 13, 14 | Fmoc-D-Phe-OH (3.0 eq) | HATU (2.85 eq) and DIEA (6.0 eq) |
| 9 | Fmoc-Hyp(4-OH)(tBu)-OH (3.0 eq) | HATU (2.85 eq) and DIEA (6.0 eq) |
| 6, 18 | Fmoc-D-Pro-OH (3.0 eq) | HATU (2.85 eq) and DIEA (6.0 eq) |
| 10, 11 | Fmoc-Ala-OH (3.0 eq) | HATU (2.85 eq) and DIEA (6.0 eq) |
| 12, 13, 14 | Fmoc-D-Ala-OH (3.0 eq) | HATU (2.85 eq) and DIEA (6.0 eq) |
| 1 | Fmoc-hArg(Et)₂-OH (3.0 eq) | HATU (2.85 eq) and DIEA (6.0 eq) |
| 8 | Fmoc-Lys(Boc)-OH (3.0 eq) | HATU (2.85 eq) and DIEA (6.0 eq) |
| 10, 11, 12, | Fmoc-Ser(tBu)-OH (3.0 eq) | HATU (2.85 eq) and DIEA (6.0 eq) |
| 13, 14 | | |
| 15, 16, 17, | Fmoc-Arg(Pbf) (6.00 eq) | HATU (5.70 eq) and DIEA (12.0 eq) |
| 18, 19, 20 | | |
| 10, 11, 12, | Fmoc-Gly-OH (3.0 eq) | HATU (2.85 eq) and DIEA (6.0 eq) |
| 13, 14 | | |
| 1, 2, 9, 15, | Fmoc-Asn(Trt)-OH (3.0 eq) | HATU (2.85 eq) and DIEA (6.0 eq) |
| 20 | | |
| 10, 11, 12, | Fmoc-Asn(Trt)-OH (6.0 eq) | HATU (5.70 eq) and DIEA (6.0 eq) |
| 13, 14 | | |
| 1, 2 | Fmoc-His(Trt)-OH (3.0 eq) | HATU (2.85 eq) and DIEA (6.0 eq) |
| 10, 11, 12, | Fmoc-His(Trt)-OH (6.0 eq) | HATU (5.70 eq) and DIEA (6.0 eq) |
| 13, 14 | | |
| 1, 2, 15, 16, | Fmoc-Ala-OH (3.0 eq) | HATU (2.85 eq) and DIEA (6.0 eq) |
| 19, 20 | | |
| 10, 11 | Fmoc-Phg-OH (3.0 eq) | DIC (3.0 eq) and HOBt (3.0 eq) |
| 1, 2 | Fmoc-AOC-OH (2.0 eq) | HBTU (1.90 eq) and DIEA (4.0 eq) |
| 15, 16, 19, | Fmoc-AOC-OH (3.0 eq) | HBTU (2.85 eq) and DIEA (6.0 eq) |
| 20 | | |
| 10, 11 | Fmoc-NLE-OH (2.0 eq) | HBTU (1.90 eq) and DIEA (4.0 eq) |
| 12, 13, 14 | Fmoc-NLE-OH (2.0 eq) | HBTU (2.85 eq) and DIEA (6.0 eq) |

### LCMS Properties of Purify Peptides Represented by Examples 1-20

| Peptide | LCMS details (Method B) |
|---|---|
| No. | (Observed mass ion, retention time) |
| 1 | 995.2 [M + 2H]²⁺, 1.40 min |
| 2 | 960.1 [M + 2H]²⁺, 1.38 min |
| 3 | 637.1 [M + 3H]³⁺, 1.38 min |
| 4 | 637.2 [M + 3H]³⁺, 1.38 min |
| 5 | 955.2 [M + 2H]²⁺, 1.38 min |
| 6 | 955.2 [M + 2H]²⁺, 1.38 min |
| 7 | 955.1 [M + 2H]²⁺, 1.38 min |
| 8 | 941.0 [M + 2H]²⁺, 1.40 min |
| 9 | 816.3 [M + 2H]²⁺, 1.39 min |
| 10 | 816.4 [M + 2H]²⁺, 1.45 min |
| 11 | 799.4 [M + 2H]²⁺, 1.49 min |
| 12 | 748.9 [M + 2H]²⁺, 1.44 min |
| 13 | 765.9 [M + 2H]²⁺, 1.47 min |
| 14 | 748.9 [M + 2H]²⁺, 1.43 min |
| 15 | 894.1 [M + 2H]²⁺, 1.38 min |
| 16 | 901.6 [M + 2H]²⁺, 1.38 min |
| 17 | 689.1 [M + 3H]³⁺, 1.38 min |
| 18 | 689.1 [M + 3H]³⁺, 1.38 min |
| 19 | 689.2 [M + 3H]³⁺, 1.41 min |
| 20 | 684.6 [M + 3H]³⁺, 1.41 min |

### Example 3. Orexin Type 2 Receptor Agonist Activity of Exemplary Peptides

Stable cell line generation. Obtainment of cells stably expressing either human orexin type 2 or human orexin type 1 receptor: to obtain a stable cell line the Orexin receptor cDNA was inserted into pcDNA3.1(+) plasmid vector and clones identified by G418 drug resistance selection. Clones demonstrating functional activity Orexin A were selected and taken into continuous culture. A single clone for OX2R-CHO and OX1R-CHO were grown in bulk and frozen to generate a cell bank for routine screening.

Measurement of orexin type 1 receptor agonist activity. Chinese hamster ovary (CHO) cells expressing human orexin type 1 receptor (hOX1R) were seeded in each well of 384 well black clear bottom plates (BD Flacon) at 10,000 cells per well and cultured for 24 h in an Ham's F12 (Gibco) medium containing 10% fetal calf serum (Sigma Aldrich) under the conditions of 37° C., 5% CO₂. After removal of the medium, 50 μL of assay buffer 1 (0.1% bovine serum albumin (Sigma Aldrich), 20 mM HEPES (Molecular Dimensions), 250 mM probenecid (Sigma Aldrich), 1× Calcium 5 dye (Molecular Devices) in Hank's balanced salt solution (Invitrogen)) was added, and the cells were incubated for 60 min under the conditions of 37° C., 5% CO₂. A test peptide was dissolved in dimethyl sulfoxide (Sigma Aldrich) to 1 mM, and then diluted with assay buffer 2 (20 mM HEPES, Hank's balanced salt solution, 0.1% bovine serum albumin). For the reaction, a test peptide solution (10 μl) was added using Fluorescent Imaging Plate Reader TETRA (FLIPR TETRA: manufactured by Molecular Devices), a fluorescence value (excitation wavelength 488 nm, measurement wavelength 570 nm) of each well was measured every one second for 2 min, and the agonist activity was determined using the area of the fluorescence value as an indicator of intracellular Ca²⁺ concentration. The agonist activity of the test peptide was calculated assuming that the fluorescence value of the well added with only the dilution buffer was 0% and the fluorescence value of the well added with 10 nM human orexin A (Tocris) buffer was 100%. The agonist activity values EC₅₀ and Eₘₐₓ of each peptide are shown in Table 1 below. As used herein, Eₘₐₓ indicates the value at 1 μM concentration when orexin A is converted to a full agonist (maximum value of agonist activity: 100%).

Measurement of orexin type 2 receptor agonist activity. Chinese hamster ovary (CHO) cells expressing human orexin type 2 receptor (hOX2R) seeded in each well of 384 well black clear bottom plates (BD Flacon) at 10,000 cells per well and cultured for 24 h in an Ham's F12 (Gibco) medium containing 10% fetal calf serum (Sigma Aldrich) under the conditions of 37° C., 5% CO₂. After removal of the medium, 50 μL of assay buffer 1 (0.1% bovine serum albumin (Sigma Aldrich), 20 mM HEPES (Molecular Dimensions), 250 mM probenecid (Sigma Aldrich), 1× Calcium 5 dye (Molecular Devices) in Hank's balanced salt solution (Invitrogen)) was added, and the cells were incubated for 60 min under the conditions of 37° C., 5% CO₂. A test peptide was dissolved in dimethyl sulfoxide (Sigma Aldrich) to 1 mM, and then diluted with assay buffer 2 (20 mM HEPES, Hank's balanced salt solution, 0.1% bovine serum albumin). For the reaction, a test peptide solution (10 μl) was added using Fluorescent Imaging Plate Reader TETRA (FLIPR TETRA: manufactured by Molecular Devices), a fluorescence value (excitation wavelength 488 nm, measurement wavelength 570 nm) of each well was measured every one second for 2 min, and the agonist activity was determined using the area of the fluorescence value as an indicator of intracellular Ca²⁺ concentration. The agonist activity of the test peptide was calculated assuming that the fluorescence value of the well added with only the dilution buffer was 0% and the fluorescence value of the well added with 10 nM human orexin A (Tocris) buffer was 100%. The agonist activity values EC₅₀ and Eₘₐₓ of each peptide are shown in Table A below. As used herein, Eₘₐₓ indicates the value at 1 μM concentration when orexin A is converted to a full agonist (maximum value of agonist activity: 100%).

Values of hOX1 and hOX2 pEC₅₀ in Table A are presented in ranges, in which “+”<8.0, 8.0≤“++”<9.0 9.0<“+++”<10.0, and 10.0<“++++”.

Values of hOX1 and hOX2 Eₘₐₓ in Table A are presented in ranges, in which 40<“F”<50, 50<“E”<60, 60≤“D”<70, 70≤“C”<80, 80≤“B”<90, and 90≤“A”.

TABLE A

| Peptide No. | hOX1 pEC₅₀ | hOX1 Eₘₐₓ | hOX2 pEC₅₀ | hOX2 Eₘₐₓ |
|---|---|---|---|---|
| 1 | + | A | ++++ | A |
| 2 | + | A | ++++ | A |
| 3 | + | A | ++++ | A |
| 4 | + | A | ++++ | A |
| 5 | + | A | ++++ | A |
| 6 | + | A | ++++ | A |
| 7 | + | A | ++++ | A |
| 8 | + | A | ++++ | A |
| 9 | + | A | ++++ | A |
| 10 | + | A | ++++ | A |
| 11 | + | A | ++++ | A |
| 12 | + | A | ++++ | A |
| 13 | + | A | ++++ | A |
| 14 | + | A | ++++ | A |
| 15 | ++ | A | ++++ | A |
| 16 | + | A | ++++ | A |
| 17 | + | A | ++++ | A |
| 18 | + | A | ++++ | A |
| 19 | + | A | ++++ | A |
| 20 | ++ | A | ++++ | A |

### Example 4. Activity Comparison of 13 Amino Acid Sequences and 14/15 Amino Acid Sequences

The peptide examples described herein were compared against sequence listings with 13 amino acids, for example:

(SEQ ID NO: 7)
CAP-TRP-ALA-SER-GLY-ASN-HIS-ALA-ALA-
GLY-ILE-LEU-THR-NLE-NH₂;
and
(SEQ ID NO: 8)
TRP-ARG-SER-GLY-ASN-HIS-ALA-Phg-GLY-
ILE-LEU-THR-NLE-NH₂

The two above sequences are representative of a set of 13 amino acid sequences that were tested for both OX1 and OX2 activity. Both SEQ ID NO:7 and SEQ ID NO:8 showed OX1 pEC₅₀ below 6.5 and OX2 pEC₅₀ below 8.8. All of the 13 amino acid sequences tested showed OX1 pEC₅₀ below 7.8 and OX2 pEC₅₀ below 9.6. Thus, in contrast to the 14 and 15 amino acid peptides of the instant disclosure (e.g., Peptide Nos. 1-20), none of the 13 amino acid sequences met the potency criteria for further development.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. (canceled)

2. (canceled)

3. A peptide having a sequence comprising:
Z₁X₁X₂X₃X₄X₅X₆X₇X₈X₉X₁₀X₁₁X₁₂X₁₃X₁₄X₁₅-NH₂
(Construct Number 3),
Z₁ is absent, a capping group, Arg, or hArg(Et)₂;
X₁ is absent, Phe, Leu, Gln, D-Gln, or Hyp(4-OH);
X₂ is Gln, Asn, N-Me-Asn, Thr, Ser, D-Gln, D-Leu, Leu, hArg, D-hArg, or Hyp(4-OH);
X₃ is Arg, hArg(Et)₂, Hyp(4-OH), His, Lys, Asp, Glu, Ser, D-Phe, Trp, hArg, Pro, D-Pro, or D-hArg;
X₄ is Hyp(4-OH), His, Arg, Lys, Asp, Gln, Glu, Pro, D-Pro, Ala, or D-Ala;
X₅ is Arg, hArg(Et)₂, Hyp(4-OH), His, Lys, Asp, Glu, or Ser;
X₆ is N-Phenethyl-Gly, N-(naphthalen-2-yl-ethyl)-Gly, N-(naphthalen-1-yl-ethyl)-Gly, N-(3-EtNH₂-Phenethyl)-Gly, N-(4-OMe-Phenethyl)-Gly, Phg, Phg(4-OH), or Gly, wherein the phenyl of X₆ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₇ is Asn, N-Me-Asn, Ser, Thr, or Gln;
X₈ is Hyp(4-OH), His, Arg, Lys, Asp, Gln, or Glu;
X₉ is Ala, Val, Ile, Leu, Met, Phe, Tyr, or Trp, wherein the phenyl of X₉ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₀ is Phg, Phg(4-OH), N-Phenethyl-Gly, N(naphtha-2-yl-ethyl)-Gly, N-(4-OMe-Phenethyl)-Gly, Gly, or Ala, wherein the phenyl of X₁₀ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₁ is Phg, Phg(4-OH), N-Phenethyl-Gly, N(naphtha-2-yl-ethyl)-Gly, N-(4-OMe-Phenethyl)-Gly, or Gly, wherein the phenyl of X₁ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₂ is Ile, Ala, Val, Leu, Met, Phe, Tyr, or Trp, wherein the phenyl of X₁₂ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₃ is N-Me-Leu, Leu, Ala, Val, Ile, Met, Phe, Tyr, or Trp, wherein the phenyl of X₁₃ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl;
X₁₄ is Thr, Ser, Asn, or Gln; and
X₁₅ is 2-AOC, 2-AHP, NLE, NVA, Phe, or Phe(3-Br), wherein the phenyl of X₁₅ is optionally substituted with halo, —OH, —O(C₁-C₆ alkyl), —CN, —NH₂, —NH(C₁-C₆ alkyl), —N(C₁-C₆ alkyl)₂, C₁-C₆ alkyl, C₂-C₆ alkenyl, or C₂-C₆ alkynyl.

4-45. (canceled)

46. The peptide of claim 3, wherein Z₁ is absent or CAP.

47. The peptide of claim 3, wherein X₁ is absent, Phe, Leu, Gln, D-Gln, or Hyp(4-OH).

48. The peptide of claim 3, wherein X₂ is Gln, D-Gln, Hyp(4-OH), D-Leu, hArg, D-hArg, or Leu.

49. The peptide of claim 3, wherein X₃ is Hyp(4-OH), D-Phe, Trp, hArg, Pro, D-Pro, or D-hArg.

50. The peptide of claim 3, wherein X₄ is Hyp(4-OH), Arg, Pro, D-Pro, Ala, or D-Ala.

51. The peptide of claim 3, wherein X₅ is Arg, hArg(Et)₂, Lys, or Ser.

52. The peptide of claim 3, wherein X₇ is Asn or N-Me-Asn.

53. The peptide of claim 3, wherein X₈ is Hyp(4-OH) or His.

54. The peptide of claim 3, wherein X₉ is Ala.

55. The peptide of claim 3, wherein X₁₀ is Phg or Ala.

56. The peptide of claim 3, wherein X₁₁ is Gly.

57. The peptide of claim 3, wherein X₁₂ is Ile.

58. The peptide of claim 3, wherein X₁₃ is Leu.

59. The peptide of claim 3, wherein X₁₄ is Thr.

60. The peptide of claim 3, wherein X₁₅ is 2-AOC or NLE.

61. The peptide of claim 3, wherein the peptide is selected from a peptide represented by the structure of: 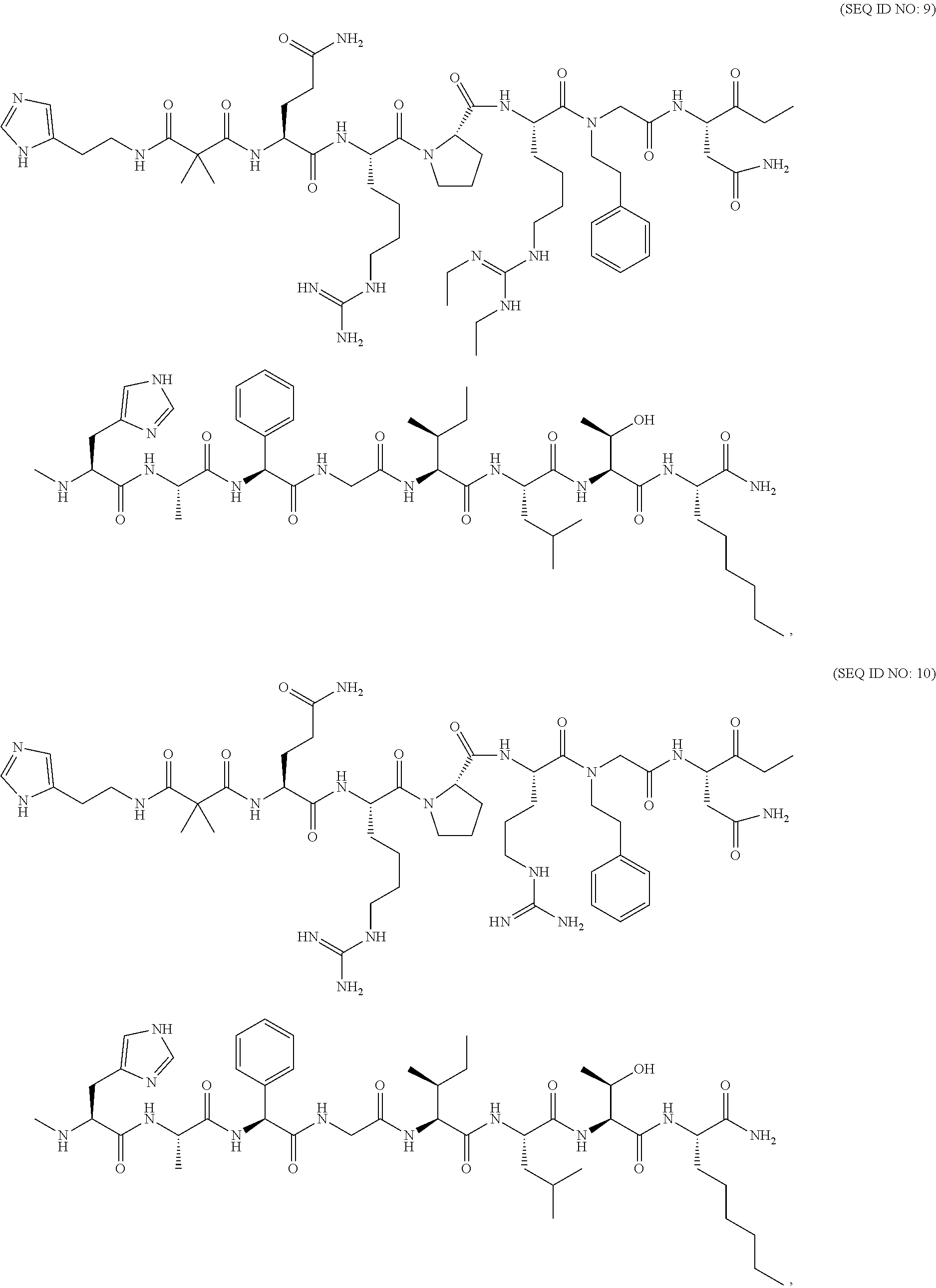 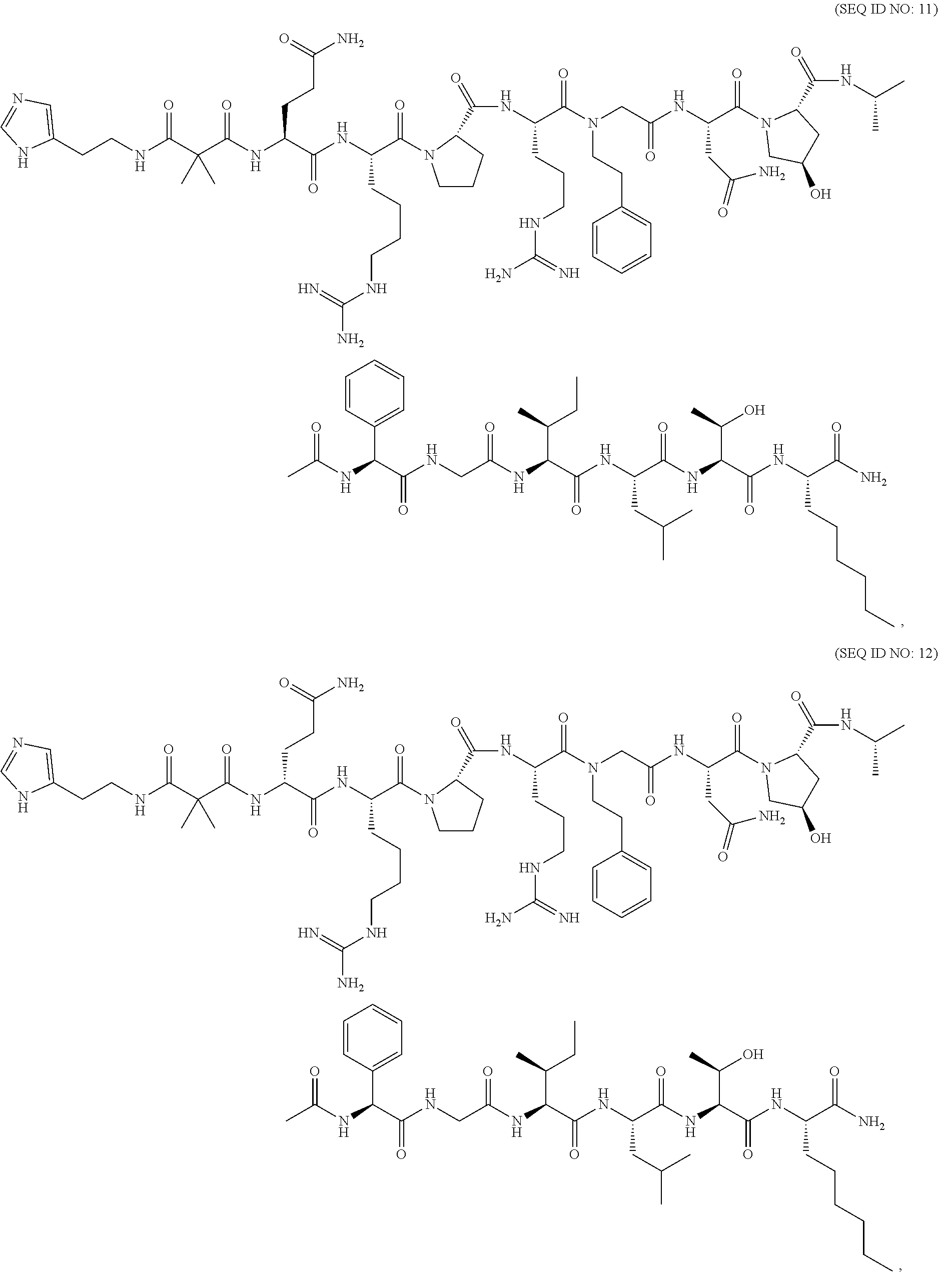 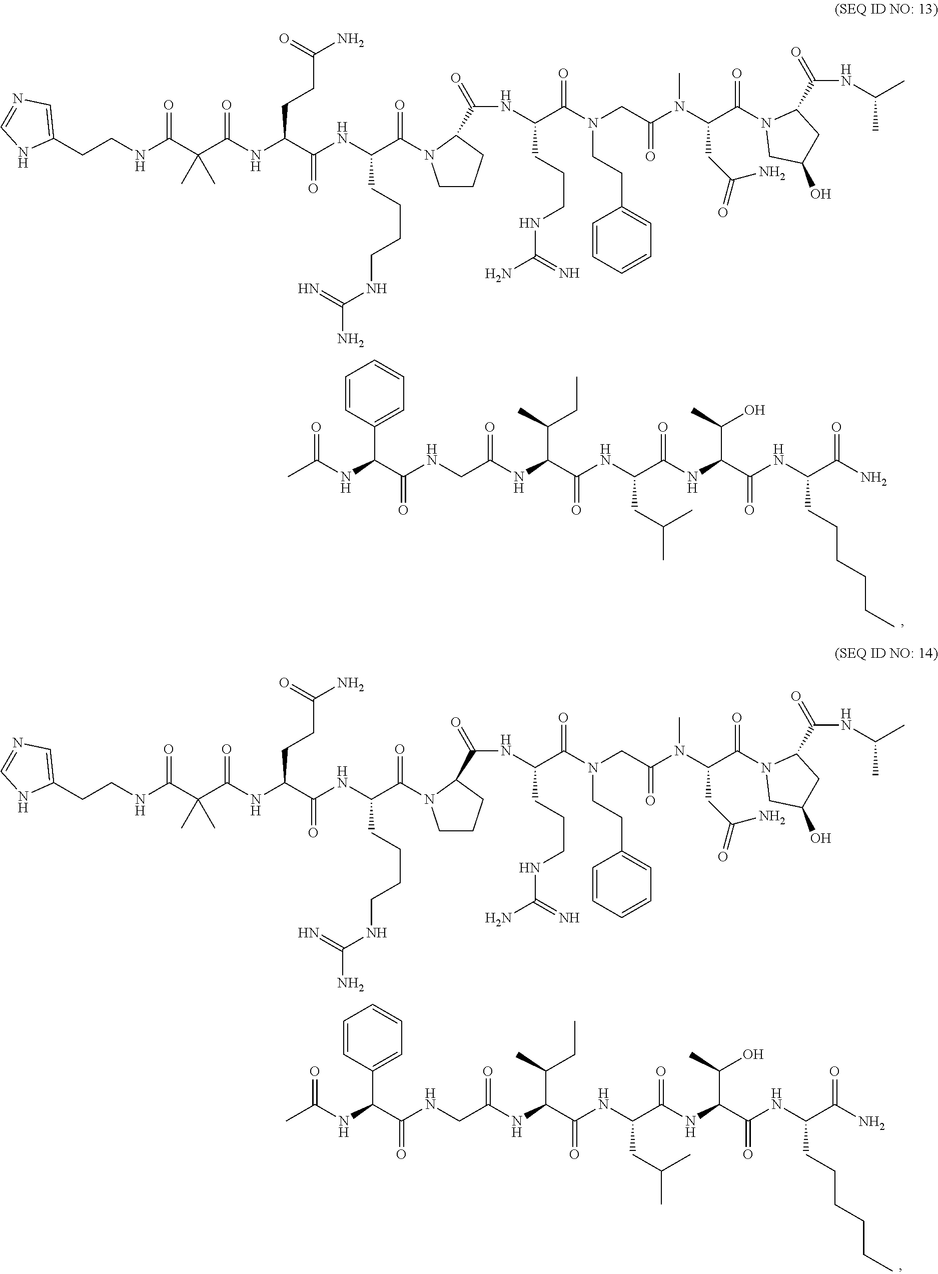 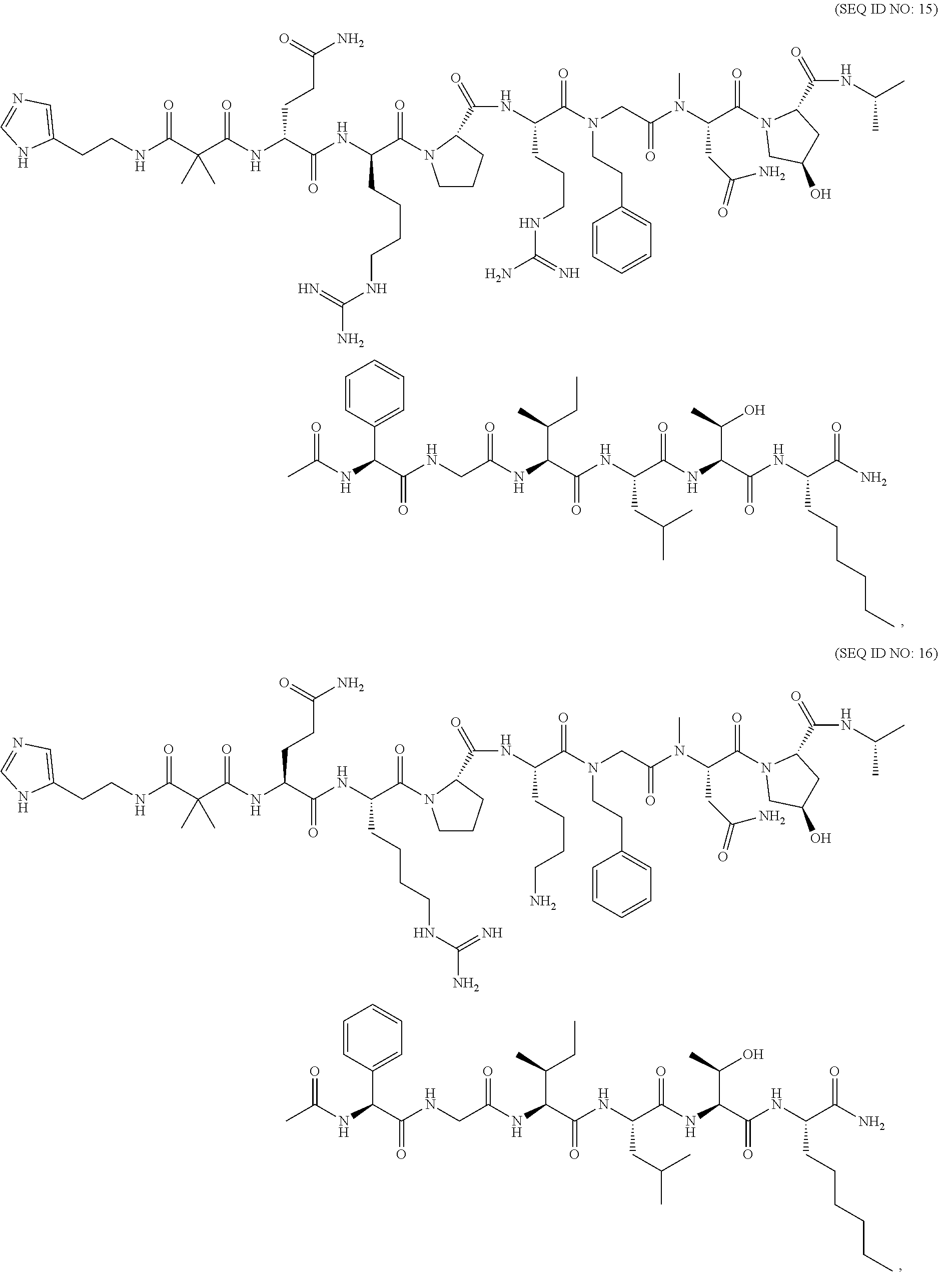 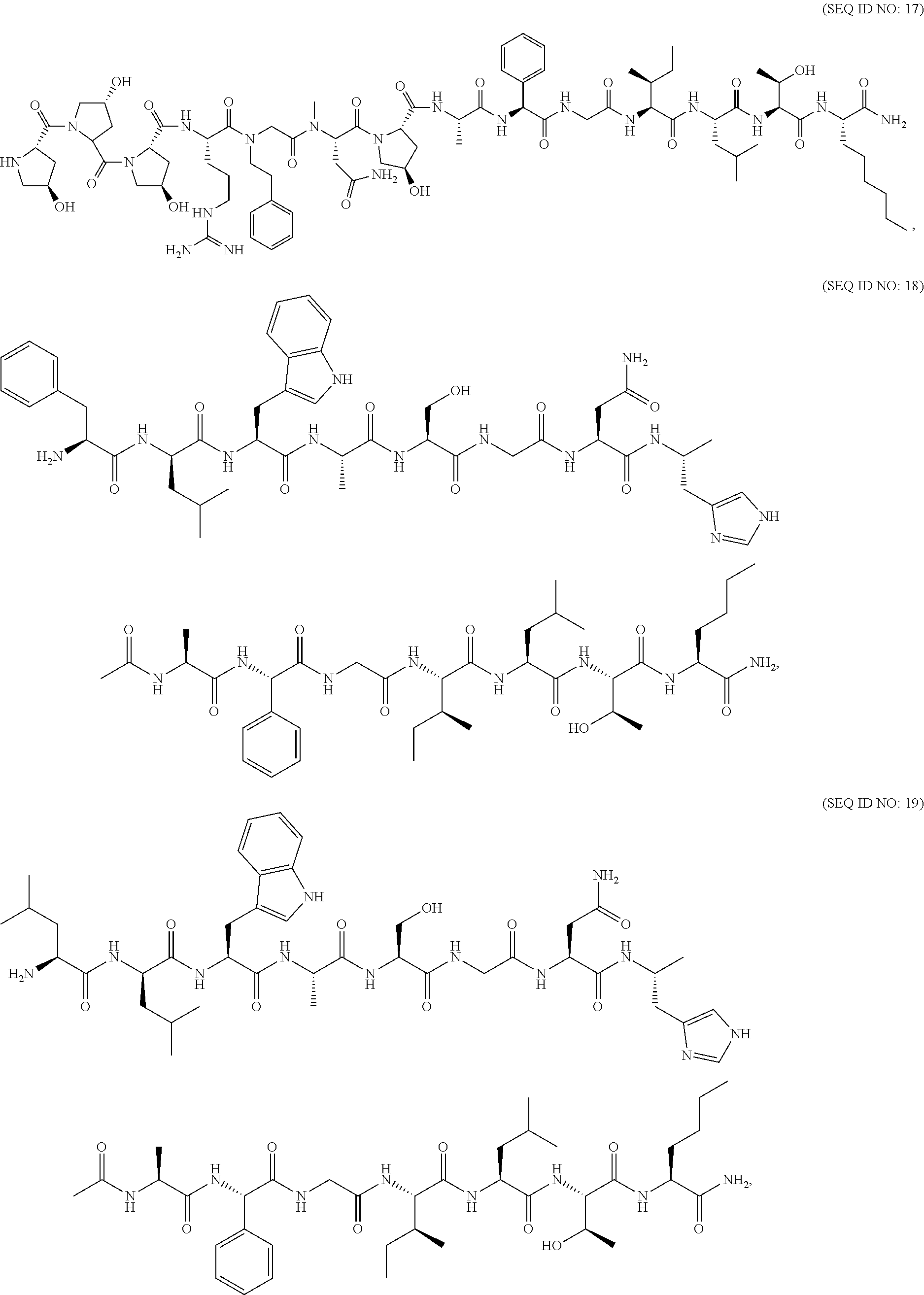 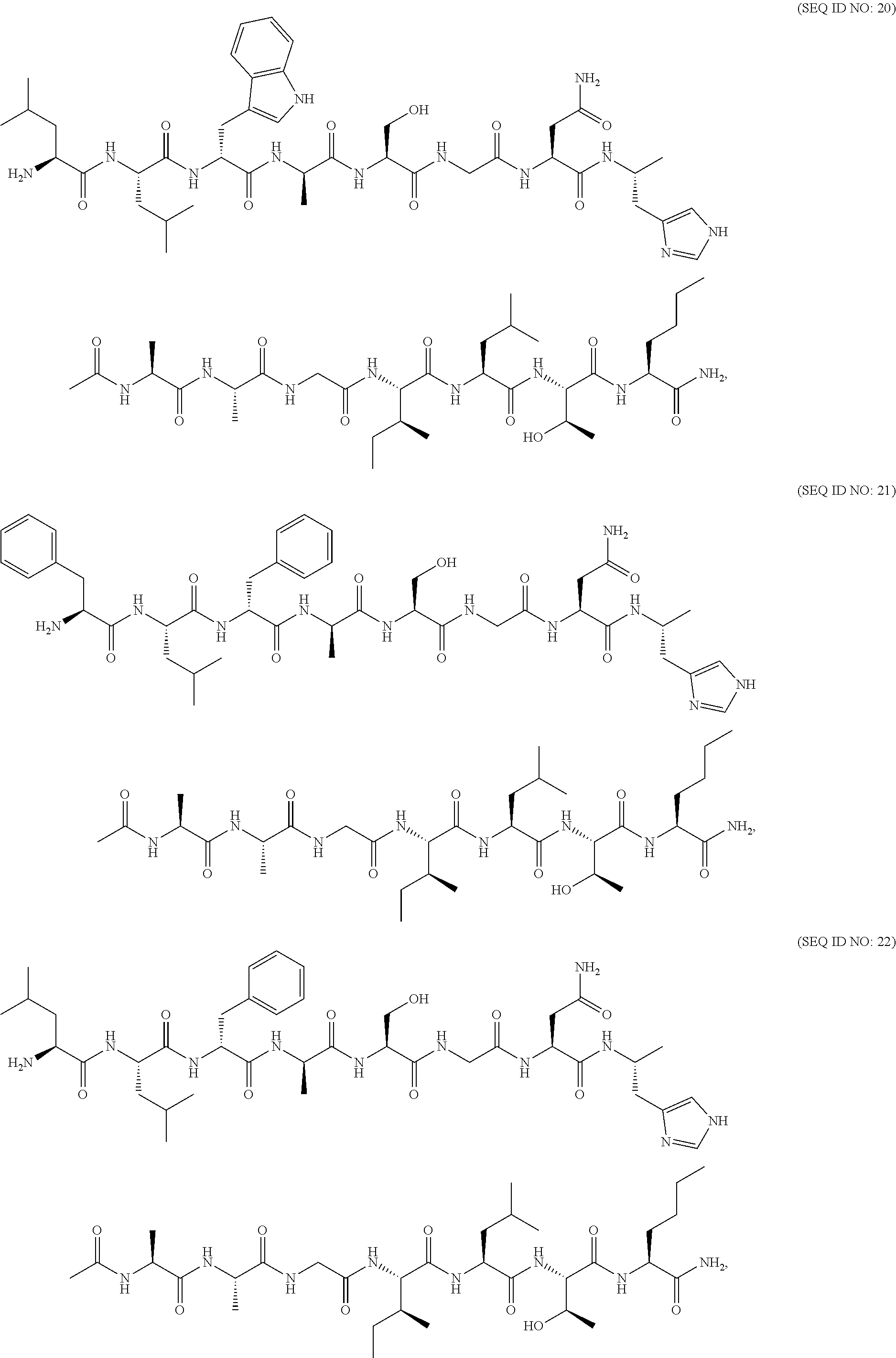 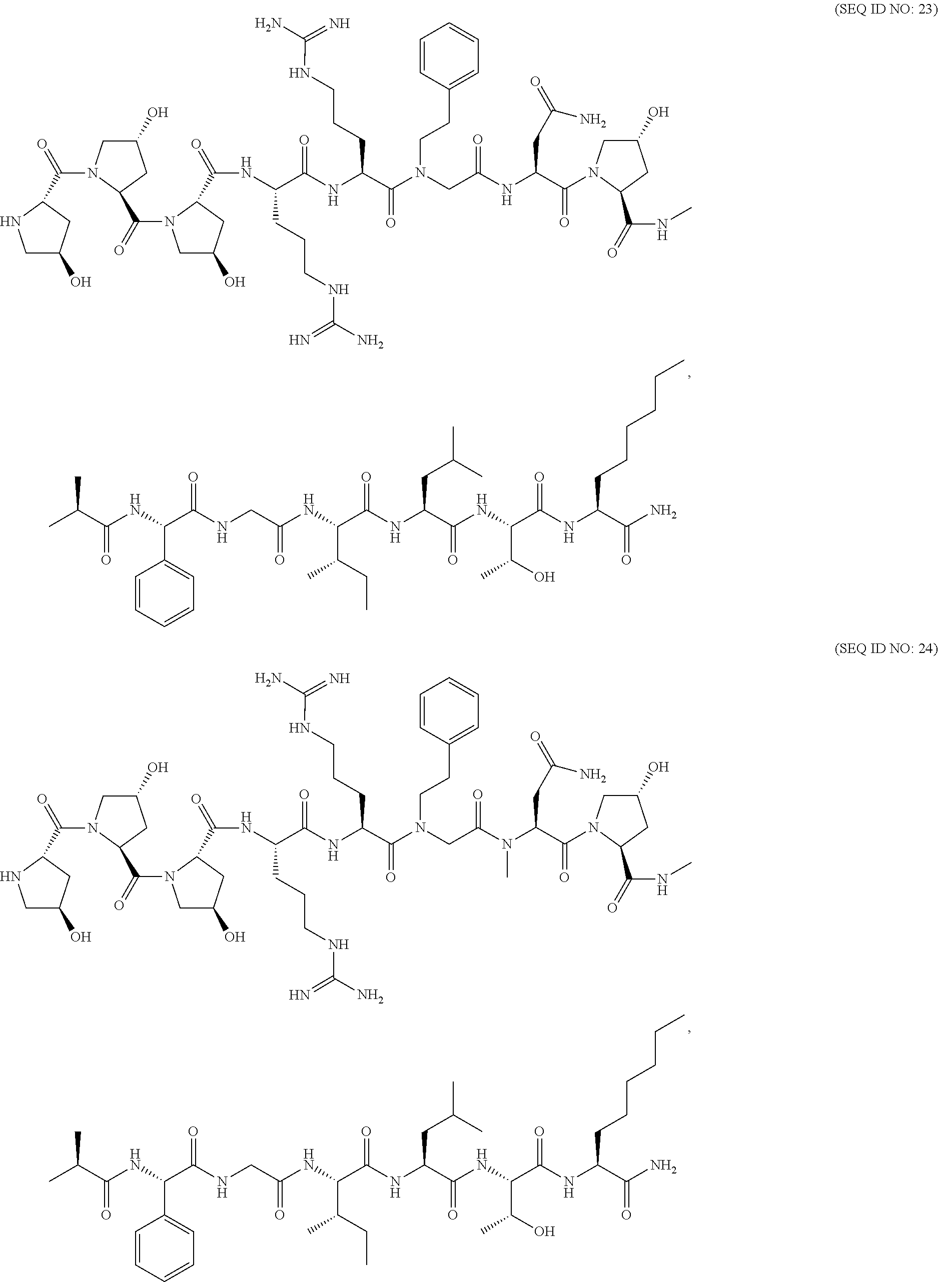 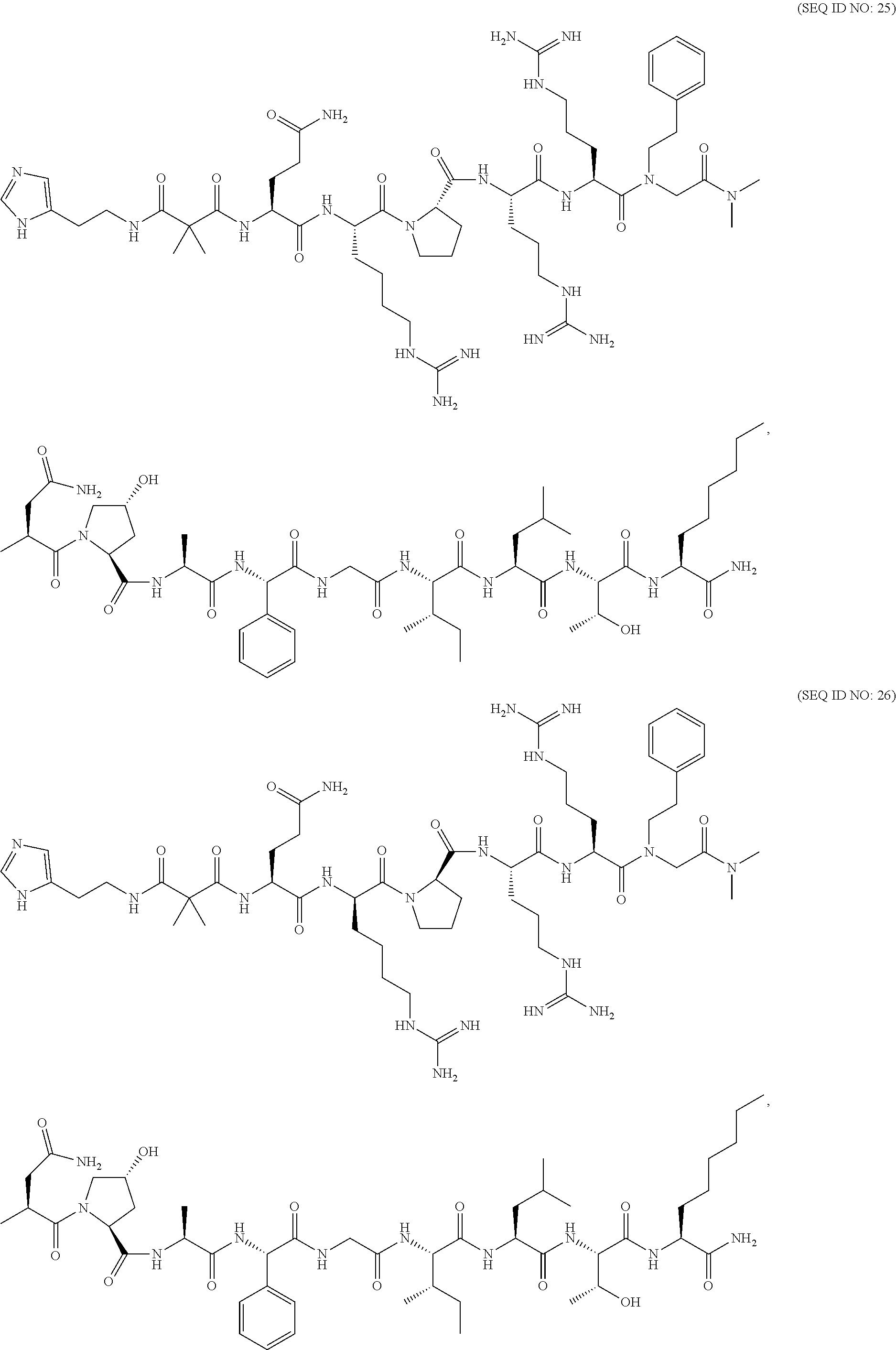 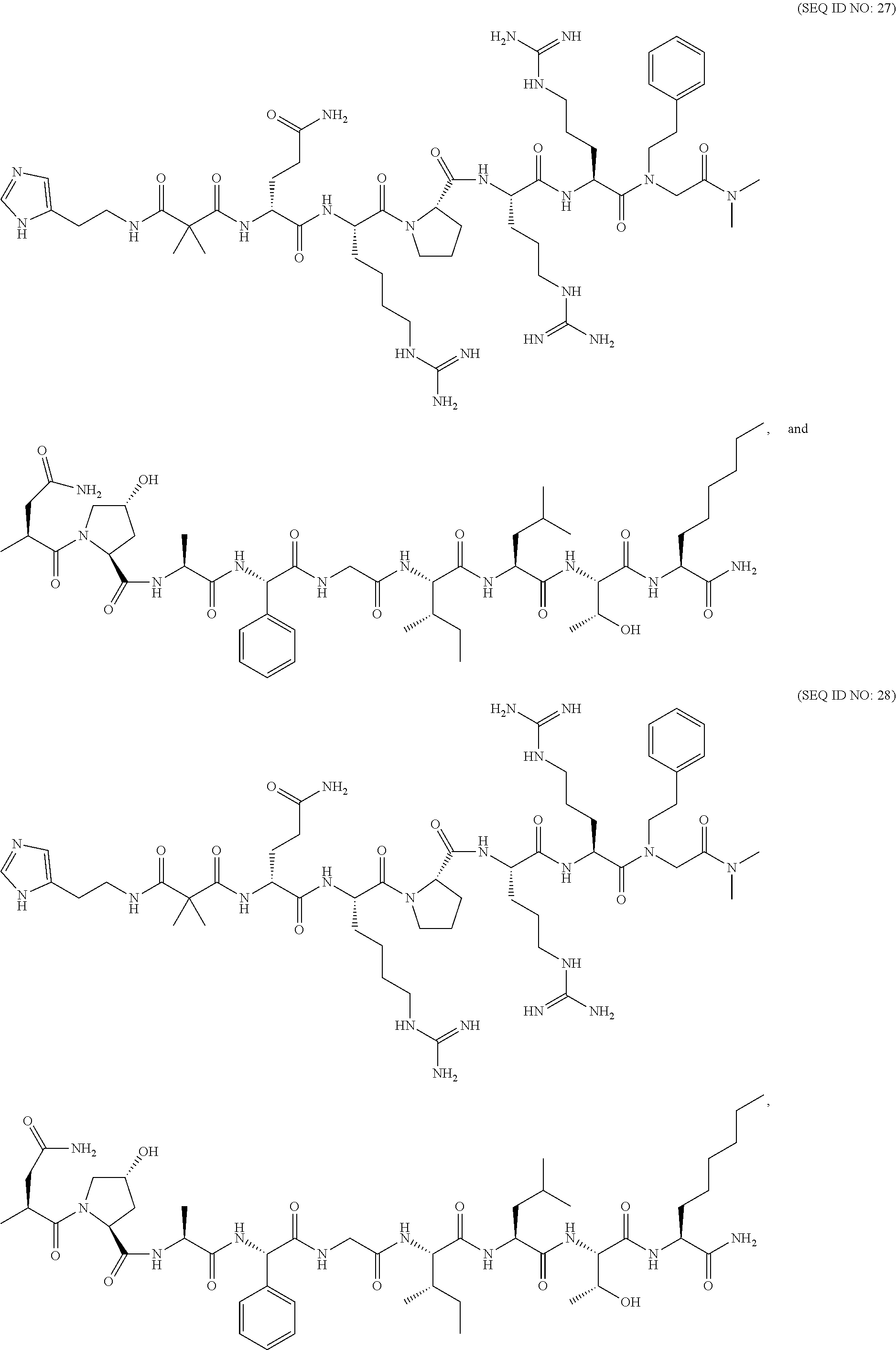
or a pharmaceutically acceptable salt of any one thereof.

62. A pharmaceutical composition comprising the peptide of claim 3, or a pharmaceutically acceptable salt thereof, and a pharmaceutically acceptable diluent or carrier.

63. A method of treating or preventing a disease or disorder in a subject, comprising administering to the subject a peptide of claim 3 or a pharmaceutically acceptable salt thereof.

64. The method of claim 63, wherein the disease or disorder is narcolepsy, a hypersomnia disorder, a neurodegenerative disorder, a neurological disorder, a symptom of a rare genetic disorder, a psychiatric disorder, a mental health disorder, a circadian rhythm disorder, a metabolic syndrome, osteoporosis, cardiac failure, coma, or a complication in emergence from anesthesia.


SEQUENCE LISTING (28 sequences)

SEQ ID NO: 1-4 - skipped (4 entries)

SEQ ID NO: 5 - protein, 16 aa, synthetic construct
  VARIANT 1: note = Arg, Diethyl homoarginine, or absent
  VARIANT 2: note = Any amino acid comprising a hydrophobic side chain, or a derivative thereof, or a polar uncharged side chain, or a derivative thereof, or absent
  VARIANT 3: note = Any amino acid comprising a polar uncharged side chain, or a derivative thereof, a hydrophobic side chain, or a derivative thereof, or an electrically charged side chain, or a derivative thereof
  VARIANT 4: note = Any amino acid comprising an electrically charged side chain, or a derivative thereof, a polar uncharged side chain, or a derivative thereof, or a hydrophobic side chain, or a derivative thereof
  VARIANT 5: note = Any amino acid comprising a polar charged side chain, or a derivative thereof, or a hydrophobic side chain, or a derivative thereof
  VARIANT 6: note = Any amino acid comprising an electrically charged side chain, or a derivative thereof, or a polar uncharged side chain, or a derivative thereof
  VARIANT 7: note = Gly or a Gly derivative
  VARIANT 8: note = Any amino acid comprising a polar uncharged side chain, or a derivative thereof
  SITE 9: note = 4-hydroxy-L-proline
  SITE 11: note = Phenylglycine
  VARIANT 16: note = Any amidated amino acid comprising a hydrophobic side chain or a derivative thereof
XXXXXXXXXAXGILTX

SEQ ID NO: 6 - protein, 15 aa, synthetic construct
  VARIANT 1: note = Any amino acid comprising a hydrophobic side chain, or a derivative thereof, or a polar uncharged side chain, or a derivative thereof, or absent
  VARIANT 2: note = Any amino acid comprising a polar uncharged side chain, or a derivative thereof, a hydrophobic side chain, or a derivative thereof, or an electrically charged side chain, or a derivative thereof
  VARIANT 3: note = Any amino acid comprising an electrically charged side chain, or a derivative thereof, a polar uncharged side chain, or a derivative thereof, or a hydrophobic side chain, or a derivative thereof
  VARIANT 4: note = Any amino acid comprising a polar charged side chain, or a derivative thereof, or a hydrophobic side chain, or a derivative thereof
  VARIANT 5: note = Any amino acid comprising an electrically charged side chain, or a derivative thereof, or a polar uncharged side chain, or a derivative thereof
  VARIANT 6: note = Gly or a Gly derivative
  VARIANT 7: note = Any amino acid comprising a polar uncharged side chain, or a derivative thereof
  SITE 8: note = 4-hydroxy-L-proline
  SITE 10: note = Phenylglycine
  SITE 15: note = Amidated 2-aminooctanoic acid
  SITE 1: note = H2N-(PEG)6 capping group
XXXXXXXXAXGILTX

SEQ ID NO: 7 - protein, 13 aa, synthetic construct
  SITE 13: note = Amidated norleucine
  SITE 1: note = 3-((2-(1H-imidazol-5-yl)ethyl)amino)-2,2-dimethyl-3 -oxopropanoyl group (CAP) capping group
WASGNHAAGILTX

SEQ ID NO: 8 - protein, 13 aa, synthetic construct
  SITE 8: note = Phenylglycine
  SITE 13: note = Amidated norleucine
WRSGNHAXGILTX

SEQ ID NO: 9 - protein, 14 aa, synthetic construct
  SITE 2: note = Homoarginine
  SITE 4: note = Diethyl homoarginine
  SITE 5: note = N-Phenethyl glycine
  SITE 9: note = Phenylglycine
  SITE 14: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPXGNHAXGILTX

SEQ ID NO: 10 - protein, 14 aa, synthetic construct
  SITE 2: note = Homoarginine
  SITE 5: note = N-Phenethyl glycine
  SITE 9: note = Phenylglycine
  SITE 14: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRGNHAXGILTX

SEQ ID NO: 11 - protein, 14 aa, synthetic construct
  SITE 2: note = Homoarginine
  SITE 5: note = N-Phenethyl glycine
  SITE 6: note = N-methyl asparagine
  SITE 7: note = 4-hydroxy-L-proline
  SITE 9: note = Phenylglycine
  SITE 14: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRGNXAXGILTX

SEQ ID NO: 12 - protein, 14 aa, synthetic construct
  SITE 1: note = D-amino acid
  SITE 2: note = Homoarginine
  SITE 5: note = N-Phenethyl glycine
  SITE 6: note = N-methyl asparagine
  SITE 7: note = 4-hydroxy-L-proline
  SITE 9: note = Phenylglycine
  SITE 14: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRGNXAXGILTX

SEQ ID NO: 13 - protein, 14 aa, synthetic construct
  SITE 2: note = D-Homoarginine
  SITE 5: note = N-Phenethyl glycine
  SITE 6: note = N-methyl asparagine
  SITE 7: note = 4-hydroxy-L-proline
  SITE 9: note = Phenylglycine
  SITE 14: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRGNXAXGILTX

SEQ ID NO: 14 - protein, 14 aa, synthetic construct
  SITE 2: note = Homoarginine
  SITE 3: note = D-amino acid
  SITE 5: note = N-Phenethyl glycine
  SITE 6: note = N-methyl asparagine
  SITE 7: note = 4-hydroxy-L-proline
  SITE 9: note = Phenylglycine
  SITE 14: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRGNXAXGILTX

SEQ ID NO: 15 - protein, 14 aa, synthetic construct
  SITE 1: note = D-amino acid
  SITE 2: note = D-Homoarginine
  SITE 5: note = N-Phenethyl glycine
  SITE 6: note = N-methyl asparagine
  SITE 7: note = 4-hydroxy-L-proline
  SITE 9: note = Phenylglycine
  SITE 14: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRGNXAXGILTX

SEQ ID NO: 16 - protein, 14 aa, synthetic construct
  SITE 2: note = Homoarginine
  SITE 5: note = N-Phenethyl glycine
  SITE 6: note = N-methyl asparagine
  SITE 7: note = 4-hydroxy-L-proline
  SITE 9: note = Phenylglycine
  SITE 14: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPKGNXAXGILTX

SEQ ID NO: 17 - protein, 14 aa, synthetic construct
  SITE 1: note = 4-hydroxy-L-proline
  SITE 2: note = 4-hydroxy-L-proline
  SITE 3: note = 4-hydroxy-L-proline
  SITE 5: note = N-Phenethyl glycine
  SITE 7: note = 4-hydroxy-L-proline
  SITE 9: note = Phenylglycine
  SITE 14: note = Amidated 2-aminooctanoic acid
XXXRGNXAXGILTX

SEQ ID NO: 18 - protein, 15 aa, synthetic construct
  SITE 2: note = D-amino acid
  SITE 10: note = Phenylglycine
  SITE 15: note = Amidated norleucine
FLWASGNHAXGILTX

SEQ ID NO: 19 - protein, 15 aa, synthetic construct
  SITE 2: note = D-amino acid
  SITE 10: note = Phenylglycine
  SITE 15: note = Amidated norleucine
LLWASGNHAXGILTX

SEQ ID NO: 20 - protein, 15 aa, synthetic construct
  SITE 3: note = D-amino acid
  SITE 4: note = D-amino acid
  SITE 15: note = Amidated norleucine
LLFASGNHAAGILTX

SEQ ID NO: 21 - protein, 15 aa, synthetic construct
  SITE 3: note = D-amino acid
  SITE 4: note = D-amino acid
  SITE 15: note = Amidated norleucine
FLFASGNHAAGILTX

SEQ ID NO: 22 - protein, 15 aa, synthetic construct
  SITE 2: note = D-amino acid
  SITE 3: note = D-amino acid
  SITE 4: note = D-amino acid
  SITE 15: note = Amidated norleucine
LLFASGNHAAGILTX

SEQ ID NO: 23 - protein, 15 aa, synthetic construct
  SITE 1: note = 4-hydroxy-L-proline
  SITE 2: note = 4-hydroxy-L-proline
  SITE 3: note = 4-hydroxy-L-proline
  SITE 6: note = N-Phenethyl glycine
  SITE 8: note = 4-hydroxy-L-proline
  SITE 10: note = Phenylglycine
  SITE 15: note = Amidated 2-aminooctanoic acid
XXXRRGNXAXGILTX

SEQ ID NO: 24 - protein, 15 aa, synthetic construct
  SITE 1: note = 4-hydroxy-L-proline
  SITE 2: note = 4-hydroxy-L-proline
  SITE 3: note = 4-hydroxy-L-proline
  SITE 6: note = N-Phenethyl glycine
  SITE 7: note = N-methyl asparagine
  SITE 8: note = 4-hydroxy-L-proline
  SITE 10: note = Phenylglycine
  SITE 15: note = Amidated 2-aminooctanoic acid
XXXRRGNXAXGILTX

SEQ ID NO: 25 - protein, 15 aa, synthetic construct
  SITE 2: note = Homoarginine
  SITE 6: note = N-Phenethyl glycine
  SITE 7: note = N-methyl asparagine
  SITE 8: note = 4-hydroxy-L-proline
  SITE 10: note = Phenylglycine
  SITE 15: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRRGNXAXGILTX

SEQ ID NO: 26 - protein, 15 aa, synthetic construct
  SITE 1: note = D-amino acid
  SITE 2: note = D-Homoarginine
  SITE 3: note = D-amino acid
  SITE 6: note = N-Phenethyl glycine
  SITE 7: note = N-methyl asparagine
  SITE 8: note = 4-hydroxy-L-proline
  SITE 10: note = Phenylglycine
  SITE 15: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRRGNXAXGILTX

SEQ ID NO: 27 - protein, 15 aa, synthetic construct
  SITE 1: note = D-amino acid
  SITE 2: note = Homoarginine
  SITE 6: note = N-Phenethyl glycine
  SITE 7: note = N-methyl asparagine
  SITE 8: note = 4-hydroxy-L-proline
  SITE 10: note = Phenylglycine
  SITE 15: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRRGNXAXGILTX

SEQ ID NO: 28 - protein, 15 aa, synthetic construct
  SITE 1: note = D-amino acid
  SITE 2: note = Homoarginine
  SITE 6: note = N-Phenethyl glycine
  SITE 8: note = 4-hydroxy-L-proline
  SITE 10: note = Phenylglycine
  SITE 15: note = Amidated 2-aminooctanoic acid
  SITE 1: note = 3-((2-(1H-imidazol-5-yl)ethyl)amino)-2,2-dimethyl-3 -oxopropanoyl group (CAP) capping group
QXPRRGNXAXGILTX